SEQUENCE LISTING (103 sequences)

SEQ ID NO: 1 - DNA, 507 bp, synthetic construct
  misc_feature 1..507: note = GLuc (Gaussia princeps Luc)
  source 1..507: mol_type = other DNA
atgaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgac

SEQ ID NO: 2 - DNA, 513 bp, synthetic construct
  misc_feature 1..513: note = NLuc (NanoLuc)
  source 1..513: mol_type = other DNA
atggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccctatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcg

SEQ ID NO: 3 - DNA, 444 bp, synthetic construct
  misc_feature 1..444: note = TLuc (TurboLuc16)
  source 1..444: mol_type = other DNA
atggaagccgaggccgagagaggcaagctgcccggcaaaaagctgcccctggaagtgctgatcgagctggaagccaacgccagaaaggccggctgcaccagaggctgcctgatctgcctgagcaagatcaagtgcaccgccaagatgaagaagtacatccccggcagatgcgccgactacggcggcgataagaaaacaggccaggccggcatcgtgggagccatcgtggatatccctgagatcagcggcttcaaagaaatggaacccatggaacagtttatcgcccaggtggaccgctgcgccgattgcacaacaggctgtctgaagggcctggctaacgtgaagtgcagcgacctgctgaagaagtggctgcctggcagatgtgccaccttcgccgacaagatccagagcgaggtggacaacatcaagggactggccggcgac

SEQ ID NO: 4 - DNA, 459 bp, synthetic construct
  misc_feature 1..459: note = MLuc7 (Metrida longa Luc7) M43L/M110L variant
  source 1..459: mol_type = other DNA
atgaaccccaccgtgaacaacgacgtgaaccggggcaagatgcccggcaagaaactgcccctggaagtgctgatcgagctggaagccaacgccttcaaggccggctgcaccagaggctgcctgatctgcctgagcaagatcaagtgtaccgccaagatgaagcagtacatccccggcagatgccacgactacggcggcgacaagaaaacaggccaggccggaatcgtgggagccatcgtggacatccctgagatcagcggcttcaaagagatggaacccctggaacagtttatcgcccaggtggacctgtgcgccgattgcacaaccggctgtctgaagggcctggctaacgtgaagtgcagcgagctgctgaagaagtggctgcccgacagatgcgccagcttcgccgacaagatccagaaagaggcccacaacatcaagggactggccggcgacaga

SEQ ID NO: 5 - DNA, 618 bp, synthetic construct
  misc_feature 1..618: note = LoLuc (Lucicutia ovaliformis Luc)
  source 1..618: mol_type = other DNA
atgctgcctgccagccccaccgacagatccatcgtgctggacaacggctacgtgtgcagctgggagggcatccccgacgacctgagagactgccccaagaccgaggacatgagcaagcagcatggcgctgccctgaagctgccccccgatgtgctggacgagatggaatgcaacgccaagaaaagcggctgcgtgcggggctgcctccagtgtctggccctgattaagtgcaccgccaagatgcggaagtacatccccggcagatgccacagctacgagggcgacaaggacattgcccagggcggcatcggcaaagagctgaccatcgacatccccgagatccccggcttcctggacctggcccctatggatcagttcgtggcccaggtggacctgtgcgtggactgctccagcagatgcctgaagggcctggctaacgtgcagtgctcctgcaagctgtataagtggctgcccaccagatgcaccggcttccaggccaagatcaagaaagaggccgacaccgtgatcggactggaagatgccctggccctgggcttcgacaccatccaggcttgtgtggccgctggcaagtgcaaggacaccgtgggcagatacagc

SEQ ID NO: 6 - DNA, 507 bp, synthetic construct
  misc_feature 1..507: note = HtLuc (Heterorhabdus tanneri Luc)
  source 1..507: mol_type = other DNA
atggccgcctctgaagaggccgatgacgatcacgtgtccctcgtgaagaactactggcggatcggcgtgggcaacgagcgggatgtgtctctggatagaggcggccctcccaagctgagcaaagaactgctggccgagatgcacgccattgccagcaatgccggctgctccagagtgtgcctgatcggcctgagcaagatcaagtgcacccccaagatgaagaccttcctgcccggcagatgcaacaccttcgcccctaaacctgccaccggcgacggaccttttgccgccgctgctgccatccctggcttcagcgatctgaccgctatggaacagtacaaggcccaggtggcccagtgcgactgctccaacagatgcctcgtgggcctggctaacatcaagtgttctgccgccctgaaggccgccctgcctcagagatgtaccacctttgccaccaacatccagaaagagggcgaggtggacagcatcaagggctacggcagaaag

SEQ ID NO: 7 - DNA, 585 bp, synthetic construct
  misc_feature 1..585: note = PaLuc1 (Pleuromamma abdominalis Luc1)
  source 1..585: mol_type = other DNA
atgcagcccaccgagaacaagcaggaaagccagatcgaggacatcgacagaagcaccagcctgggcctgatgtgctacgagcagtgtacaggccagagcggcctggatctgaagtgctacaaagagtgcgccgacttcaccggcgaccggaacagaggcagaaagctgcccggcaagaaactgcccctggaagtgctgaagatcatggaagccaacgccagaagggccggctgcaccagaggctgcctgatctgcctgagcaagatcaagtgcaccgccaagatgaagcagtacatccccggcagatgccacacctacgagggcgacaagtctatcggccagggcggcatcggaggccccatcgtggatatccctgagatcatcggcttccagaacatggaacctatggaacagtttatcgcccaggtggacctgtgcgccgattgcacaaccggctgtctgaagggcctggctaacgtgcggtgcaacgacctgctgaagaagtggctgcccgacagatgcgccggcttcgccaacaagatccagagcgaggtggacaacatcaagggactggccggcgacaga

SEQ ID NO: 8 - DNA, 510 bp, synthetic construct
  misc_feature 1..510: note = PaLuc2 (Pleuromamma abdominalis Luc2)
  source 1..510: mol_type = other DNA
atgaagagcatagacagctacgaaaatattgatatagtggctgttgcgggcaacttcgctgctgttgaccaggacgctaaccgcggaggaaacctgccgggtaagaaaatgccaatagaggttcttaaagaaatggaagctaacgccaagcgagcagggtgtgttcggggctgtttgatatgcttgtcccacatcaaatgcacagctaaaatgaaaaaattcataccaggtcggtgtcatagctatcatggagatgcagatacgaagcaaggggcacttgaagaagtggttgacatgcccgagataccgggatttgtggacatggagcccatggaacagtttatagcccaggtggataaatgcgaggactgtaccactggttgtttgaaaggtctcgccaacgtccactgcagcgatctcctcaagaaatggctgccgcaaagatgctcacagtttgcggataagatccaatccgaggtggacacaataaagggcctggcaggggaccgg

SEQ ID NO: 9 - DNA, 579 bp, synthetic construct
  misc_feature 1..579: note = MpLuc1[Metridia pacifica Luc1]
  source 1..579: mol_type = other DNA
atgaacccgacggagaacaaggacgatattgatattgtgggagtggaaggcaaattcgggactacggatcttgaaactgatctctttacgattgttgaggacatgaacgtaatctcaagggatacgaacctggcaaatagtgacgcagaccgaggcaaaatgccgggaaaaaaacttccgttggaagtactgatcgagatggaagcgaatgcacgcaaggcagggtgtacacggggatgcttgatctgtctgagtaagattaaatgcaccgctaaaatgaaagtttatatacccggtcggtgccacgattatgggggcgataagaaaaccggtcaggcagggatcgtcggagcaatcgtggatattcctgaaatttcaggattcaaagaacttggaccaatggagcaatttatagcacaagtggacctctgcgctgactgcacgacgggatgtctgaaaggtcttgcaaatgtaaagtgttcagcactccttaagaaatggcttcccgaccgatgtgcctcttttgctgacaagattcagtctgaagttgataacataaagggtcttgcgggtgatcgg

SEQ ID NO: 10 - DNA, 510 bp, synthetic construct
  misc_feature 1..510: note = McLuc1 [Metridia curticauda Luc1]
  source 1..510: mol_type = other DNA
atgaagcctaccgaaaataatgatgatattgatatagttgggatcgcaagtacatttatcacgacaaacaccgacgcggaccggggcaagatgccgggaaagaaacttcccttggcagtcctcaaggaaatggaggcgaacgctgcaaaggcagggtgttccagaggttgtctgatctgcttgtctaaaatcaagtgtacagctaagatgaagcagtttattccggggcgctgtcacgactacggtggtgataaaaaaacgggccaggcggcgctggtcggagcaatattcgacatccctgaaatcttcggatttctcgatatggaacctatcgagcagttcatagcacaagtagatttgtgtgctggctgcacaacaggctgcttgaaaggactcgcaaatattaagtgttctgaactcttgaagaagtggcttccgaaaagatgtacctccttcgcgtataaaatgcaaaaggaaatgcacaatataaaaggcatggcaggagatcgg

SEQ ID NO: 11 - DNA, 510 bp, synthetic construct
  misc_feature 1..510: note = MaLuc1 [Metridia asymmetrica Luc1]
  source 1..510: mol_type = other DNA
atgaaagcgaccgaaaacaatgacgacatagacatagttggaatcgcgtcaaccttcataacgacaaacacggacgccgaccgaggcaaaatgccggggaaaagattgccactggcggtgttgaaagagatggaagcgaacgcagtcaaagcgggatgtagcaggggctgccttatatgtctgagtaagattaaatgcaccgcgaagatgaagcaatacatccccggcaggtgccacgattacggaggagacaagaagaccgggcaggcggctatagagggcgcgatagatgatattcctgaaatctctggctttaaggagatggcgcctatggagcaattcattgcgcaggttgacttgtgcgcagactgcacgacaggatgcctcaagggcttggcaaatgtaaaatgttccgaacttctgaaaaaatggctgcccaagaggtgtacctccttcgccacgaagatgcaaaaggagatccataacatcaaaggcatggggggggaccgc

SEQ ID NO: 12 - DNA, 588 bp, synthetic construct
  misc_feature 1..588: note = MoLuc1 [Metridia okhotensis Luc1]
  source 1..588: mol_type = other DNA
atgaaccctacggagacccaggacggagtggacattctcggcgtggaagggaaattcgggacagaaaccaacttggagactgatctgtttaccatttgggagataaatggaataattaaatctgatcgcgacacaaatagggcaaatgcagacgctgatcgaggaaaaatgcccggcaagaaacttcctttggcggtgctcatagagatggaggcaaacgcattcaaagcgggttgcactcgcggttgtctcatttgtttgtccaaaatcaaatgcacggctaagatgaaagagtatatcccaggacggtgccatgattacggtggggataaaaaaacgggacaagcaggtatagtaggtgctatcgtggacattccagaaatctcaggatttaaagagcttggccccatggagcaattcatagcgcaagtagacctttgcgctgattgcactactggctgtcttaagggactggcgaatgttaagtgcagcgccctgttgaagaagtggctgccagaccgatgcgcatcattcgcggataagatacagtccgaagtccataatataaaaggtctcgcaggtgatcgc

SEQ ID NO: 13 - DNA, 522 bp, synthetic construct
  misc_feature 1..522: note = MoLuc2 [Metridia okhotensis Luc2]
  source 1..522: mol_type = other DNA
atggccacaatcaatgaaaatttcgaagacatcgaccttgtcgcgattggaggctcatttgctacagatgtggacgccaacaggggtggtcatggtggacatccgggcaaaaagatgcctaaagaggtcttgttggaaatggaagcgaacgcaaaacgagccggatgtcataggggttgtttgatttgtctttctcacatcaaatgtacgcagaagatgaagaaattcataccgggacggtgtcactcctatgctggtgacaaggatagcgctcagggcggcataacagaggaggagactgttgacatgcccgagatcgctgggttcaaggatcttgaacctatggagcagtttattgcacaggtagacttgtgcgttgactgcacaactggttgtttgaaagggctggccaacgtccactgttctgacttgctcaaaaagtggctgccttcccggtgcaagacttttgcctcaaaaattcagagtcaggttgacaccatcaaggggttggctggtgacagg

SEQ ID NO: 14 - DNA, 579 bp, synthetic construct
  misc_feature 1..579: note = MLuc39 [Metridia longa Luc39]
  source 1..579: mol_type = other DNA
atgaatcctacagagaacaatgatcatatcaatattgtcgggatcgagggtaaattcggaatcacagacttggagacggatctcttcactatctgggagacaaatcgcatgatttctaccgataacgaacaggcaaatacagacagtaatagagggaagatgccaggtaagaaactgccattggcagttcttatagagatggaagccaacgctttcaaggcgggttgtacaagaggctgtctcatttgcttgtctaaaatcaagtgtactgctaagatgaagaaatacataccgggaagatgccatgactacggaggtgataagaagactgggcaggcaggaatcgtgggcgcgattgtcgacatacccgacatatccgggttcaaggaaatgggaccgatggagcaattcatcgcccaagtggatcgatgcaccgactgcacaacgggatgtttgaagggtcttgcaaatgttaagtgctccgagctgttgaagaagtggttgccggaccgctgcgcgagtttcgccgataaaattcaaagtgaggtgcataatattaaaggtctggcgggagatcgc

SEQ ID NO: 15 - DNA, 498 bp, synthetic construct
  misc_feature 1..498: note = PsLuc1 [Pleuromamma scutullata Luc1]
  source 1..498: mol_type = other DNA
atgcaacccactgagaataaaaaggaatcctataccgaagacacagacgtaaacggggaccatgatcgaggcaggaagcttccggggaagaaacttccactggaagttctgaaaatcatggaagctaacgcaaggagagcaggttgcacgcgaggctgtttgatatgtcttagcaagataaaatgtactgcaaaaatgaagcaatacataccgggaagatgccatacttatgagggggacaaaagcatagggcaggcaggcataggaggcccaatcattgatataccggaaattatagggtttaagaacatggaaccaatggaacagttcatcgcacaggtggatctctgtgcggactgcactactggatgcctcaaaggtctcgcaaatgtaagatgcaacgatcttttgaaaaaatggttgccagatcgatgcgcggggttcgcactcaagatacaaggtgaagtggaaaacataaaggggatggccggggatcga

SEQ ID NO: 16 - DNA, 618 bp, synthetic construct
  misc_feature 1..618: note = LoLuc1-3 [Lucicutia ovaliformis Luc1-3]
  source 1..618: mol_type = other DNA
atgcttccagcgtcacccaccgataggtcaatagtccttgacaacggctatgtgtgttcctgggagggtatcccagacgacctgcgggactgtccgaagacagaggatatgtctaagcagcacggagctgctctcaaactgccgccagatgtgttggatgagatggaatgtaatgctaaaaagagtggatgcgtgcgaggatgtcttcagtgtctggcactcataaaatgtactgcaaaaatgagaaagtatatccccggacgctgtcatagctacgagggtgacaaggacattgctcagggggggatcggaaaggaactgacaattgacatccctgagattcctggcttcttggatcttgcaccaatggaccagttcgtggcgcaggtcgacctctgcgtggactgcagcagtcggtgcctgaagggcttggctaatgtccaatgcagttgtaagctctacaaatggttgccaacccggtgtaccggcttccaagcgaaaataaagaaagaagctgacacggtaattgggctggaagatgcgctcgcactcggattcgacaccatccaggcatgcgtagcggccggtaaatgcaaggatacggttggccggtactct

SEQ ID NO: 17 - DNA, 534 bp, synthetic construct
  misc_feature 1..534: note = HtLuc2 [Heterorhabdus tanneri Luc 2]
  source 1..534: mol_type = other DNA
atgtgggcggcctccgaggaggccgatgacgacctcgtatctttggtcaaaaactactggggcgtaggcgtaagcaatgaacgcgatgtatctctcgacagggggggccatggcaagctgccgaaaaagcttagcgttgagattctcgctgaaatggaagcaaacgcccagaagagtaattgctctcgggggtgtctgatagggctttctaaaattaaatgcacgcccaaaatgaaaaaattcctccctggcaggtgccacgaatactctggtgatcccaaaactggacagggtccgcttactgcggctgcggttataccagggtatagtgatcttacagctatggagcagttcaagctccaagtagacaagtgtgactgttcaactcaatgtcttaagggtcttgcaaatgtcaagtgctcagcggccctcaaagcggtgttgccaacaagatgctcacaattcgcaactcagatccaagctgaagtaggcaccataaaagggaaggggaagaaaccgactccaccgataggt

SEQ ID NO: 18 - DNA, 579 bp, synthetic construct
  misc_feature 1..579: note = Lucia-Luc
  source 1..579: mol_type = other DNA
atgaaacccactgaaatcaatgaagacctcaatatagctgctgtggcctccaactttgccaccacagatcttgagactgacctgttcaccaactgggagaccatgaatgtgattagcactgacacagagcaggtgaacacagatgctgacaggggcaagctgcctggcaaaaaactccccccagatgtcctgagggagctggaggccaatgccagaagggctggttgcacaagaggctgcctcatttgcctctcccacattaagtgcacccctaagatgaagaaatttatccctggcaggtgccacacttatgaaggtgaaaaggagtctgctcagggagggattggagaggcaattgttgatatcccagagattcctggcttcaaggataaggagccactggaccagtttattgctcaagtggacctctgtgctgattgcaccactggctgtctgaagggccttgccaatgtccagtgctctgacctcctgaagaagtggcttccccagaggtgtaccacttttgccagcaagattcagggtagggtggacaaaatcaagggtctggctggggacaga

SEQ ID NO: 19 - DNA, 933 bp, synthetic construct
  misc_feature 1..933: note = RLuc (Renilla Luc)
  source 1..933: mol_type = other DNA
atggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcag

SEQ ID NO: 20 - DNA, 1650 bp, synthetic construct
  misc_feature 1..1650: note = Fluc or FfLuc (Firefly Luc)
  source 1..1650: mol_type = other DNA
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtg

SEQ ID NO: 21 - DNA, 1632 bp, synthetic construct
  misc_feature 1..1632: note = LucPPe-146-1H2
  source 1..1632: mol_type = other DNA
atggccgacaagaacatcctgtacggccccgagcctttctaccctctggaagatggaacagccggcgagcagatgttcgacgccctgagcagatatgccgccattcctggatgtatcgccctgacaaacgcccacaccaaagaaaacgtgctgtacgaagagttcctgaagctgagctgtcggctggccgagagcttcaagaagtacggcctgaagcagaacgacacaatcgccgtgtgcagcgagaacagcctccagttcttcctgcctgtgatcgccagcctgtacctgggcattattgtggcccctgtgaacgacaagtacatcgagagagagctgatccacagcctgggcatcgtgaagccccggatcgtgttctgctccaagaacaccttccagaaggtgctgaacgtcaagagcaagctgaagtccatcgagacaatcatcatcctggacctgaacgaggacctcggcggctaccagtgcctgaacaacttcatcagccagaacagcgacagcaacctggacgtgaagaagttcaagccctacagcttcaaccgggacgaccaggtggcctccatcatgtttagcagcggcaccaccggactgcccaaaggcgttatgctgacccacaagaatatcgtggcccggttctctatcgctaaggaccccaccttcggcaacgccatcaatcctacaagcgctatcctgacagtgatccccttccaccacggcttcggcatgatgaccacactgggctacttcacctgtggcttcagagtggtgctgatgcacaccttcgaggaaaagctgtttctccagagcctccaggactacaaggtggaaagcaccctgctggtgcctactctgatggccttcctggctaagtctgccctggtcgagaagtacgatctgagccacctgaaagagatcgcctctggcggagcccctctgagcaaagaaatcggcgagatggtcaagaagcggttcaagctgaacttcgtgcggcaaggctatggcctgaccgagacaacaagcgccgtgctgattacccctaagggcgacgccaagcctggcagcacaggcaaaattgtgcctctgcacgccgtgaaggtggtggaccctaccacaggcaagatcctgggacctaatgagcccggcgagctgtacttcaagggacccatgattatgaagggctactacaacaacgaggaagccaccaaggccattatcgacaacgacggctggctgcggagcggcgatattgcctactacgacaatgacggccacttctacatcgtggacagactgaagtccctcatcaagtacaagggctatcaggtggccccagccgagatcgagggtatcctgctgcaacacccctatatcgtggatgccggcgtgacaggcatccctgatgaagctgctggcgaacttccagcagctggcgtggtggttcagaccggcaagtacctgaatgagcagatcgtgcaggactacgtggcctctcaggtgtccacagccaaatggctgagaggcggcgtgaagttcctggacgagatcccaaagggctctaccggcaagatcgacagaaaggtgctgcggcagatgctggaaaagcacaccaatggc

SEQ ID NO: 22 - DNA, 1632 bp, synthetic construct
  misc_feature 1..1632: note = LucPPe-133-1B2
  source 1..1632: mol_type = other DNA
atggccgacaagaacatcctgtacggccccgagcctttctaccctctggaagatggaacagccggcgagcagatgttcgacgccctgagcagatacgccgacattcctggatgtatcgccctgacaaacgcccacaccaaagaaaacgtgctgtacgaagagttcctgaagctgagctgtcggctggccgagagcttcaagaagtacggcctgaagcagaacgacacaatcgccgtgtgcagcgagaacagcctccagttcttcctgcctgtgatcgccagcctgtacctgggcattattgtggcccctgtgaacgacaagtacatcgagagagagctgatccacagcctgggcatcgtgaagccccggatcgtgttctgctccaagaacaccttccagaaggtgctgaacgtcaagagcaagctgaagtccatcgagacaatcatcatcctggacctgaacgacgacctcggcggctaccagtgcctgaacaacttcatcagccagaacagcgacagcaacctggacgtgaagaagttcaagccctacagcttcaaccgggacgaccaggtggccctgattatgtttagcagcggcaccaccggactgcccaaaggcgttatgctgacccacaagaatatcgtggcccggttctctatcgctaaggaccccaccttcggcaacgccatcaatcctacaagcgctatcctgacagtgatccccttccaccacggcttcggcatgatgaccacactgggctacttcacctgtggcttcagagtggtgctgatgcacaccttcgaggaaaagctgtttctccagagcctccaggactacaaggtggaaagcaccctgctggtgcctactctgatggccttcctggctaagtctgccctggtcgagaagtacgatctgagccacctgaaagagatcgcctctggcggagcccctctgagcaaagaaatcggcgagatggtcaagaagcggttcaagctgaacttcgtgcggcaaggctatggcctgaccgagacaacaagcgccgtgctgattacccctaagggcgacgccaagcctggcagcacaggcaagattgtgcctttccacgccgtgaaggtggtggaccctaccaccggaaagatcctgggacctaatgagcccggcgagctgtacttcaagggacccatgattatgaagggctactacaacaacgaggaagccaccaaggccattatcgacaacgacggctggctgcggagcggcgatattgcctactacgacaatgacggccacttctacatcgtggacagactgaagtccctcatcaagtacaagggctaccaggtcgcccctgccgagattgagggtatcctgctgcaacacccctatatcgtggatgccggcgtgacaggcatccctgatgaagctgctggcgaacttccagcagctggcgtggtggttcagaccggcaagtacctgaatgagcagatcgtgcaggactacgtggcctctcaggtgtccacagccaaatggctgagaggcggcgtgatcttcctggacgagatcccaaagggctctaccggcaagatcgacagaaaggtgctgcggcagatgctggaaaagcacaccaatggc

SEQ ID NO: 23 - DNA, 1632 bp, synthetic construct
  misc_feature 1..1632: note = LucPPe-78-0B10
  source 1..1632: mol_type = other DNA
atggccgacaagaacatcctgtacggccccgagcctttctaccctctggctgatggaacagccggcgagcagatgttcgatgccctgagcagatacgccgacatcagcggatgtatcgccctgacaaacgcccacaccaaagaaaacgtgctgtacgaagagttcctgaagctgagctgtcggctggccgagagcttcaagaagtacggcctgaagcagaacgacacaatcgccgtgtgcagcgagaacggcctccagttctttctgcctgtgatcgccagcctgtacctgggcattatcgctgcccctgtgtccgataagtacatcgagagagagctgatccacagcctgggcatcgtgaagccccggatcatcttctgctccaagaacaccttccagaaggtgctgaacgtcaagagcaagctgaagtccgtggaaaccatcatcatcctggacctgaacgaggacctcggcggctaccagtgcctgaacaacttcatctctcagaacagcgacagcaacctggacgtgaagaagttcaagccctacagcttcaaccgggacgaccaggtggccctggtcatgtttagctctggcacaaccggcgtgcccaagggcgttatgctgacccacaagaatatcgtggcccggttcagcctggctaaggaccctaccttcggcaacgccatcaatcccaccaccgctatcctgacagtgatccccttccaccacggcttcggcatgatgaccacactgggctacttcacctgtggcttcagagtggtgctgatgcacaccttcgaggaaaagctgtttctccagagcctccaggactacaaggtggaaagcaccctgctggtgcctactctgatggcctttctggccaagtctgccctggtcgagaagtacgatctgagccacctgaaagagatcgcctctggcggagcccctctgagcaaagaaatcggcgagatggtcaagaagcggttcaagctgaacttcgtgcggcaaggctatggcctgaccgagacaacaagcgccgtgctgattacccctaagggcgacgctagacctggcagcacaggcaagatcgtgcctttccacgccgtgaaggtggtggaccctacaaccggcaagatcctgggacctaatgagcccggcgagctgtactttaagggcgccatgattatgaagggctactacaacaacgaggaagccaccaaggccattatcgacaacgacggctggctgcggagcggcgatattgcctactacgacaatgacggccacttctacatcgtggacagactgaagtccctcatcaagtacaagggctaccaggtcgcccctgccgagattgagggtatcctgctgcaacacccctatatcgtggatgccggcgtgacaggcatccctgatgaagctgctggcgaacttccagcagcaggcgtggtggttcagaccggcaagtacctgaatgagcagatcgtgcaggacttcgtgtccagccaggtgtccacagccaaatggctgagaggcggcgtgaagttcctggacgagatcccaaagggctccaccggaaagatcgacagaaaggtgctgcggcagatgtttgagaagcacaccaacggc

SEQ ID NO: 24 - DNA, 1632 bp, synthetic construct
  misc_feature 1..1632: note = LucPPe49-7C6A
  source 1..1632: mol_type = other DNA
atggccgacaagaacatcctgtacggccccgagcctttctaccctctggctgatggaacagccggcgagcagatgttctacgccctgagcagatacgccgacatcagcggatgtatcgccctgacaaacgcccacaccaaagaaaacgtgctgtacgaagagttcctgaagctgagctgtcggctggccgagagcttcaagaagtacggcctgaagcagaacgacacaatcgccgtgtgcagcgagaacggcctccagttcttcctgcctatcattgccagcctgtacctgggcatcattgctgcccctgtgtccgataagtacatcgagagagagctgatccactctctgggcatcgtgaagccccggatcatcttctgctccaagaacaccttccagaaggtgctgaacgtcaagagcaagctgaaatacgtggaaaccatcatcatcctggacctgaacgaggacctcggcggctaccagtgcctgaacaacttcatctctcagaacagcgacatcaacctggacgtcaagaagttcaagccctacagcttcaaccgggacgaccaggtggccctggtcatgtttagctctggcacaaccggcgtgtccaagggcgtgatgctgacccacaagaatatcgtggcccggttcagcctggctaaggaccctaccttcggcaacgccatcaatcccaccaccgctatcctgacagtgatccccttccaccacggcttcggcatgatgaccacactgggctacttcacctgtggcttcagagtggtgctgatgcacaccttcgaggaaaagctgtttctccagagcctccaggactacaaggtggaaagcaccctgctggtgcctactctgatggcctttctggctaagagcgccctggtcgagaagtacgatctgagccacctgaaagagatcgcctctggcggagcccctctgagcaaagaaatcggcgagatggtcaagaagcggttcaagctgaacttcgtgcggcaaggctatggcctgaccgagacaacaagcgccgtgctgattacccctaacaacgatgtgcggcctggcagcacaggcaagatcgtgccttttcacgccgtgaaggtggtggaccctacaaccggcaagatcctgggacctaatgagcccggcgagctgtacttcaagggcgacatgattatgaagggctactacaacaacgaggaagccaccaaggccattatcaacaaggacggctggctgcggagcggcgacattgcctactatgacaacgacggccacttctacatcgtggaccggctgaagtccctgattaagtacaagggctaccaggtcgcccctgccgagattgagggtatcctgctgcaacacccctatatcgtggatgccggcgtgacaggcatccctgatgaagctgctggcgaacttccagcagctggcgtggtggttcagaccggcaagtacctgaatgagcagatcgtgcagaatttcgtgtccagccaggtgtccaccgccaagtggcttagaggcggcgtgaagttcctggacgagatccctaaaggctccaccggaaagatcgacagaaaggtgctgcgccagatgttcgagaagcacaccaatggc

SEQ ID NO: 25 - DNA, 1626 bp, synthetic construct
  misc_feature 1..1626: note = LucPpL-81-6G1
  source 1..1626: mol_type = other DNA
atgatgaagcgcgagaagaacgtgatctacggccccgaacctctgcaccctcttgaggatcttacagccggcgagatgctgttcagagccctgcggaaacacagccatctgcctcaggctctggtggatgtcgtgggagatgagagcctgtcctacaaagagttcttcgaggccaccgtgctgctggcccagtctctgcataattgcggctacaagatgaacgacgtggtgtccatctgcgccgagaacaacacccggttcttcatccctgtgatcgccgcctggtacatcggcatgattgtggcccctgtgaacgagagctacatccccgacgagctgtgcaaagtgatgggcatcagcaagccccagatcgtgttcaccaccaagaacatcctgaacaaggtgctggaagtgcagagccggaccaacttcatcaagcggatcatcatcctggacaccgtggaaaacatccacggctgcgagagcctgccaaacggcatctccagatacagcgacggcaatatcgccaacttcaagcccctgcacttcgaccccgtggaacaggtggcagccatcctgtgtagcagcggcacaacaggactgcccaaaggcgtgatgcagacccaccagaatatctgcgtgcggctgatccacgctctggaccctagagctggaacccagctgattcctggcgtgaccgtgctggtgtacctgcctttctttcacgccttcggcttcagcatcaccctgggctacttcatggtcggactgagagtgattatgtttcggcgcttcgaccaagaagccttcctgaaggccatccaggactacgaagtgcgctccgtgattaacgtgcccagcgtgatcctgttcctgagcaagagcccactggtggataagtacgacctgagcagcctgagagagctgtgttgcggagctgcccctctggctaaagaggtggcagaagttgccgccaagcggctgaatctgcctggcatcagatgtggctttggcctgaccgagagcaccagcgccaatatccacagcctgcgggacgagtttaagagcggctctctgggcagagtgacccctctgatggccgccaagatcgccgatagagagacaggcaaagccctgggacctaatcaagtgggcgagctgtgtatcaagggacctatggtgtccaagggctacgtgaacaacgtggaagccaccaaagaggccatcgacgacgacggctggctgcactctggcgatttcggctactacgacgaggacgagcacttctacgtggtggacagatacaaagagctgattaagtacaagggcagccaggtggccccagccgagctggaagaaatcctgctgaagaacccctgcatccgcgacgtggcagttgtgggcattcctgatctggaagccggcgaactgcctagcgcctttgtggttaagcagcccggcaaagagatcaccgccaaagaggtgtacgactacctggccgagagagtgtcccacaccaagtatctgagaggcggcgtcagattcgtggacagcatccccagaaacgtgaccggcaagatcacccggaaagagctgctgaaacagctgctggaaaaggctggcgga

SEQ ID NO: 26 - DNA, 1626 bp, synthetic construct
  misc_feature 1..1626: note = CBGRluc
  source 1..1626: mol_type = other DNA
atggtaaagcgtgagaaaaatgtcatctatggccctgagcctctccatcctttggaggatttgactgccggcgaaatgctgtttcgtgctctccgcaagcactctcatttgcctcaagccttggtcgatgtggtcggcgatgaatctttgagctacaaggagttttttgaggcaaccgtcttgctggctcagtccctccacaattgtggctacaagatgaacgacgtcgttagtatctgtgctgaaaacaatacccgtttcttcattccagtcatcgccgcatggtatatcggtatgatcgtggctccagtcaacgagagctacattcccgacgaactgtgtaaagtcatgggtatctctaagccacagattgtcttcaccactaagaatattctgaacaaagtcctggaagtccaaagccgcaccaactttattaagcgtatcatcatcttggacactgtggagaatattcacggttgcgaatctttgcctaatttcatctctcgctattcagacggcaacatcgcaaactttaaaccactccacttcgaccctgtggaacaagttgcagccattctgtgtagcagcggtactactggactcccaaagggagtcatgcagacccatcaaaacatttgcgtgcgtctgatccatgctctcgatccacgctacggcactcagctgattcctggtgtcaccgtcttggtctacttgcctttcttccatgctttcggctttcatattactttgggttactttatggtcggtctccgcgtgattatgttccgccgttttgatcaggaggctttcttgaaagccatccaagattatgaagtccgcagtgtcatcaacgtgcctagcgtgatcctgtttttgtctaagagcccactcgtggacaagtacgacttgtcttcactgcgtgaattgtgttgcggtgccgctccactggctaaggaggtcgctgaagtggccgccaaacgcttgaatcttccagggattcgttgtggcttcggcctcaccgaatctaccagtgcgattatccagactctcggggatgagtttaagagcggctctttgggccgtgtcactccactcatggctgctaagatcgctgatcgcgaaactggtaaggctttgggcccgaaccaagtgggcgagctgtgtatcaaaggccctatggtgagcaagggttatgtcaataacgttgaagctaccaaggaggccatcgacgacgacggctggttgcattctggtgattttggatattacgacgaagatgagcatttttacgtcgtggatcgttacaaggagctgatcaaatacaagggtagccaggttgctccagctgagttggaggagattctgttgaaaaatccatgcattcgcgatgtcgctgtggtcggcattcctgatctggaggccggcgaactgccttctgctttcgttgtcaagcagcctggtacagaaattaccgccaaagaagtgtatgattacctggctgaacgtgtgagccatactaagtacttgcgtggcggcgtgcgttttgttgactccatccctcgtaacgtaacaggcaaaattacccgcaaggagctgttgaaacaattgttggtgaaggccggcggt

SEQ ID NO: 27 - DNA, 1470 bp, synthetic construct
  misc_feature 1..1470: note = Embryonic Alkaline Phosphatase (EAP)
  source 1..1470: mol_type = other DNA
atgatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggt

SEQ ID NO: 28 - DNA, 708 bp, synthetic construct
  misc_feature 1..708: note = mCherry
  source 1..708: mol_type = other DNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaag

SEQ ID NO: 29 - DNA, 717 bp, synthetic construct
  misc_feature 1..717: note = EGFP (Enhanced Green Fluorescent Protein)
  source 1..717: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 30 - DNA, 522 bp, synthetic construct
  misc_feature 1..522: note = MYR-GGS-GLuc (M60L/M127L variant)
  source 1..522: mol_type = other DNA
atggggagtagcaagagcaagcctaaggaccccagccagcgcagaaggggcggatctggggatcccaagcccaccgagaacaacgaagacgccgacagaggcaagctgcccggcaagaaactgcccctggaagtgctgaaagagctggaagccaacgcccggaaggccggctgtacaagaggctgcctgatctgcctgagccacatcaagtgcacgcccaagatgaagaagttcatccccggcagatgccacacctacgagggcgacaaagagtctgcccagggcggaatcggcgaggctatcgtggacattcctgagattcctgggttcaaggacctggaacccctggaacagtttatcgcccaggtggacctgtgcgtggactgcaccacaggctgtctgaagggcctggctaacgtgcagtgcagcgacctgctgaagaagtggctgccccagagatgcgccaccttcgcctctaagatccagggacaggtggacaagatcaagggggccggtggtgac

SEQ ID NO: 31 - protein, 169 aa, synthetic construct
  REGION 1..169: note = GLuc (Gaussia princeps Luc)
MKPTENNEDFNIVAVASNFATTDLDADRGKLPGKKLPLEVLKEMEANARKAGCTRGCLICLSHIKCTPKMKKFIPGRCHTYEGDKESAQGGIGEAIVDIPEIPGFKDLEPMEQFIAQVDLCVDCTTGCLKGLANVQCSDLLKKWLPQRCATFASKIQGQVDKIKGAGGD

SEQ ID NO: 32 - protein, 171 aa, synthetic construct
  REGION 1..171: note = NLuc (NanoLuc)
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 33 - protein, 148 aa, synthetic construct
  REGION 1..148: note = TLuc (TurboLuc16)
MEAEAERGKLPGKKLPLEVLIELEANARKAGCTRGCLICLSKIKCTAKMKKYIPGRCADYGGDKKTGQAGIVGAIVDIPEISGFKEMEPMEQFIAQVDRCADCTTGCLKGLANVKCSDLLKKWLPGRCATFADKIQSEVDNIKGLAGD

SEQ ID NO: 34 - protein, 153 aa, synthetic construct
  REGION 1..153: note = MLuc7 (Metrida longa Luc7) M43L/M110L variant
MNPTVNNDVNRGKMPGKKLPLEVLIELEANAFKAGCTRGCLICLSKIKCTAKMKQYIPGRCHDYGGDKKTGQAGIVGAIVDIPEISGFKEMEPLEQFIAQVDLCADCTTGCLKGLANVKCSELLKKWLPDRCASFADKIQKEAHNIKGLAGDR

SEQ ID NO: 35 - protein, 206 aa, synthetic construct
  REGION 1..206: note = LoLuc (Lucicutia ovaliformis Luc)
MLPASPTDRSIVLDNGYVCSWEGIPDDLRDCPKTEDMSKQHGAALKLPPDVLDEMECNAKKSGCVRGCLQCLALIKCTAKMRKYIPGRCHSYEGDKDIAQGGIGKELTIDIPEIPGFLDLAPMDQFVAQVDLCVDCSSRCLKGLANVQCSCKLYKWLPTRCTGFQAKIKKEADTVIGLEDALALGFDTIQACVAAGKCKDTVGRYS

SEQ ID NO: 36 - protein, 169 aa, synthetic construct
  REGION 1..169: note = HtLuc (Heterorhabdus tanneri Luc)
MAASEEADDDHVSLVKNYWRIGVGNERDVSLDRGGPPKLSKELLAEMHAIASNAGCSRVCLIGLSKIKCTPKMKTFLPGRCNTFAPKPATGDGPFAAAAAIPGFSDLTAMEQYKAQVAQCDCSNRCLVGLANIKCSAALKAALPQRCTTFATNIQKEGEVDSIKGYGRK

SEQ ID NO: 37 - protein, 195 aa, synthetic construct
  REGION 1..195: note = PaLuc1 (Pleuromamma abdominalis Luc1)
MQPTENKQESQIEDIDRSTSLGLMCYEQCTGQSGLDLKCYKECADFTGDRNRGRKLPGKKLPLEVLKIMEANARRAGCTRGCLICLSKIKCTAKMKQYIPGRCHTYEGDKSIGQGGIGGPIVDIPEIIGFQNMEPMEQFIAQVDLCADCTTGCLKGLANVRCNDLLKKWLPDRCAGFANKIQSEVDNIKGLAGDR

SEQ ID NO: 38 - protein, 170 aa, synthetic construct
  REGION 1..170: note = PaLuc2 (Pleuromamma abdominalis Luc2)
MKSIDSYENIDIVAVAGNFAAVDQDANRGGNLPGKKMPIEVLKEMEANAKRAGCVRGCLICLSHIKCTAKMKKFIPGRCHSYHGDADTKQGALEEVVDMPEIPGFVDMEPMEQFIAQVDKCEDCTTGCLKGLANVHCSDLLKKWLPQRCSQFADKIQSEVDTIKGLAGDR

SEQ ID NO: 39 - protein, 193 aa, synthetic construct
  REGION 1..193: note = MpLuc1[Metridia pacifica Luc1]
MNPTENKDDIDIVGVEGKFGTTDLETDLFTIVEDMNVISRDTNLANSDADRGKMPGKKLPLEVLIEMEANARKAGCTRGCLICLSKIKCTAKMKVYIPGRCHDYGGDKKTGQAGIVGAIVDIPEISGFKELGPMEQFIAQVDLCADCTTGCLKGLANVKCSALLKKWLPDRCASFADKIQSEVDNIKGLAGDR

SEQ ID NO: 40 - protein, 170 aa, synthetic construct
  REGION 1..170: note = McLuc1 [Metridia curticauda Luc1]
MKPTENNDDIDIVGIASTFITTNTDADRGKMPGKKLPLAVLKEMEANAAKAGCSRGCLICLSKIKCTAKMKQFIPGRCHDYGGDKKTGQAALVGAIFDIPEIFGFLDMEPIEQFIAQVDLCAGCTTGCLKGLANIKCSELLKKWLPKRCTSFAYKMQKEMHNIKGMAGDR

SEQ ID NO: 41 - protein, 170 aa, synthetic construct
  REGION 1..170: note = MaLuc1 [Metridia asymmetrica Luc1]
MKATENNDDIDIVGIASTFITTNTDADRGKMPGKRLPLAVLKEMEANAVKAGCSRGCLICLSKIKCTAKMKQYIPGRCHDYGGDKKTGQAAIEGAIDDIPEISGFKEMAPMEQFIAQVDLCADCTTGCLKGLANVKCSELLKKWLPKRCTSFATKMQKEIHNIKGMGGDR

SEQ ID NO: 42 - protein, 196 aa, synthetic construct
  REGION 1..196: note = MoLuc1 [Metridia okhotensis Luc1]
MNPTETQDGVDILGVEGKFGTETNLETDLFTIWEINGIIKSDRDTNRANADADRGKMPGKKLPLAVLIEMEANAFKAGCTRGCLICLSKIKCTAKMKEYIPGRCHDYGGDKKTGQAGIVGAIVDIPEISGFKELGPMEQFIAQVDLCADCTTGCLKGLANVKCSALLKKWLPDRCASFADKIQSEVHNIKGLAGDR

SEQ ID NO: 43 - protein, 174 aa, synthetic construct
  REGION 1..174: note = MoLuc2 [Metridia okhotensis Luc2]
MATINENFEDIDLVAIGGSFATDVDANRGGHGGHPGKKMPKEVLLEMEANAKRAGCHRGCLICLSHIKCTQKMKKFIPGRCHSYAGDKDSAQGGITEEETVDMPEIAGFKDLEPMEQFIAQVDLCVDCTTGCLKGLANVHCSDLLKKWLPSRCKTFASKIQSQVDTIKGLAGDR

SEQ ID NO: 44 - protein, 193 aa, synthetic construct
  REGION 1..193: note = MLuc39 [Metridia longa Luc39]
MNPTENNDHINIVGIEGKFGITDLETDLFTIWETNRMISTDNEQANTDSNRGKMPGKKLPLAVLIEMEANAFKAGCTRGCLICLSKIKCTAKMKKYIPGRCHDYGGDKKTGQAGIVGAIVDIPDISGFKEMGPMEQFIAQVDRCTDCTTGCLKGLANVKCSELLKKWLPDRCASFADKIQSEVHNIKGLAGDR

SEQ ID NO: 45 - protein, 166 aa, synthetic construct
  REGION 1..166: note = PsLuc1 [Pleuromamma scutullata Luc1]
MQPTENKKESYTEDTDVNGDHDRGRKLPGKKLPLEVLKIMEANARRAGCTRGCLICLSKIKCTAKMKQYIPGRCHTYEGDKSIGQAGIGGPIIDIPEIIGFKNMEPMEQFIAQVDLCADCTTGCLKGLANVRCNDLLKKWLPDRCAGFALKIQGEVENIKGMAGDR

SEQ ID NO: 46 - protein, 206 aa, synthetic construct; identical to SEQ ID NO: 35
  REGION 1..206: note = LoLuc1-3 [Lucicutia ovaliformis Luc1-3]

SEQ ID NO: 47 - protein, 178 aa, synthetic construct
  REGION 1..178: note = HtLuc2 [Heterorhabdus tanneri Luc 2]
MWAASEEADDDLVSLVKNYWGVGVSNERDVSLDRGGHGKLPKKLSVEILAEMEANAQKSNCSRGCLIGLSKIKCTPKMKKFLPGRCHEYSGDPKTGQGPLTAAAVIPGYSDLTAMEQFKLQVDKCDCSTQCLKGLANVKCSAALKAVLPTRCSQFATQIQAEVGTIKGKGKKPTPPIG

SEQ ID NO: 48 - protein, 193 aa, synthetic construct
  REGION 1..193: note = Lucia-Luc
MKPTEINEDLNIAAVASNFATTDLETDLFTNWETMNVISTDTEQVNTDADRGKLPGKKLPPDVLRELEANARRAGCTRGCLICLSHIKCTPKMKKFIPGRCHTYEGEKESAQGGIGEAIVDIPEIPGFKDKEPLDQFIAQVDLCADCTTGCLKGLANVQCSDLLKKWLPQRCTTFASKIQGRVDKIKGLAGDR

SEQ ID NO: 49 - protein, 311 aa, synthetic construct
  REGION 1..311: note = RLuc (Renilla Luc)
MASKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKIIFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGKPDVVQIVRNYNAYLRASDDLPKMFIESDPGFFSNAIVEGAKKFPNTEFVKVKGLHFSQEDAPDEMGKYIKSFVERVLKNEQ

SEQ ID NO: 50 - protein, 550 aa, synthetic construct
  REGION 1..550: note = Fluc or FfLuc (Firefly Luc)
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 51 - protein, 544 aa, synthetic construct
  REGION 1..544: note = LucPPe-146-1H2
MADKNILYGPEPFYPLEDGTAGEQMFDALSRYAAIPGCIALTNAHTKENVLYEEFLKLSCRLAESFKKYGLKQNDTIAVCSENSLQFFLPVIASLYLGIIVAPVNDKYIERELIHSLGIVKPRIVFCSKNTFQKVLNVKSKLKSIETIIILDLNEDLGGYQCLNNFISQNSDSNLDVKKFKPYSFNRDDQVASIMFSSGTTGLPKGVMLTHKNIVARFSIAKDPTFGNAINPTSAILTVIPFHHGFGMMTTLGYFTCGFRVVLMHTFEEKLFLQSLQDYKVESTLLVPTLMAFLAKSALVEKYDLSHLKEIASGGAPLSKEIGEMVKKRFKLNFVRQGYGLTETTSAVLITPKGDAKPGSTGKIVPLHAVKVVDPTTGKILGPNEPGELYFKGPMIMKGYYNNEEATKAIIDNDGWLRSGDIAYYDNDGHFYIVDRLKSLIKYKGYQVAPAEIEGILLQHPYIVDAGVTGIPDEAAGELPAAGVVVQTGKYLNEQIVQDYVASQVSTAKWLRGGVKFLDEIPKGSTGKIDRKVLRQMLEKHTNG

SEQ ID NO: 52 - protein, 544 aa, synthetic construct
  REGION 1..544: note = LucPPe-133-1B2
MADKNILYGPEPFYPLEDGTAGEQMFDALSRYADIPGCIALTNAHTKENVLYEEFLKLSCRLAESFKKYGLKQNDTIAVCSENSLQFFLPVIASLYLGIIVAPVNDKYIERELIHSLGIVKPRIVFCSKNTFQKVLNVKSKLKSIETIIILDLNDDLGGYQCLNNFISQNSDSNLDVKKFKPYSFNRDDQVALIMFSSGTTGLPKGVMLTHKNIVARFSIAKDPTFGNAINPTSAILTVIPFHHGFGMMTTLGYFTCGFRVVLMHTFEEKLFLQSLQDYKVESTLLVPTLMAFLAKSALVEKYDLSHLKEIASGGAPLSKEIGEMVKKRFKLNFVRQGYGLTETTSAVLITPKGDAKPGSTGKIVPFHAVKVVDPTTGKILGPNEPGELYFKGPMIMKGYYNNEEATKAIIDNDGWLRSGDIAYYDNDGHFYIVDRLKSLIKYKGYQVAPAEIEGILLQHPYIVDAGVTGIPDEAAGELPAAGVVVQTGKYLNEQIVQDYVASQVSTAKWLRGGVIFLDEIPKGSTGKIDRKVLRQMLEKHTNG

SEQ ID NO: 53 - protein, 544 aa, synthetic construct
  REGION 1..544: note = LucPPe-78-0B10
MADKNILYGPEPFYPLADGTAGEQMFDALSRYADISGCIALTNAHTKENVLYEEFLKLSCRLAESFKKYGLKQNDTIAVCSENGLQFFLPVIASLYLGIIAAPVSDKYIERELIHSLGIVKPRIIFCSKNTFQKVLNVKSKLKSVETIIILDLNEDLGGYQCLNNFISQNSDSNLDVKKFKPYSFNRDDQVALVMFSSGTTGVPKGVMLTHKNIVARFSLAKDPTFGNAINPTTAILTVIPFHHGFGMMTTLGYFTCGFRVVLMHTFEEKLFLQSLQDYKVESTLLVPTLMAFLAKSALVEKYDLSHLKEIASGGAPLSKEIGEMVKKRFKLNFVRQGYGLTETTSAVLITPKGDARPGSTGKIVPFHAVKVVDPTTGKILGPNEPGELYFKGAMIMKGYYNNEEATKAIIDNDGWLRSGDIAYYDNDGHFYIVDRLKSLIKYKGYQVAPAEIEGILLQHPYIVDAGVTGIPDEAAGELPAAGVVVQTGKYLNEQIVQDFVSSQVSTAKWLRGGVKFLDEIPKGSTGKIDRKVLRQMFEKHTNG

SEQ ID NO: 54 - protein, 544 aa, synthetic construct
  REGION 1..544: note = LucPPe49-7C6A
MADKNILYGPEPFYPLADGTAGEQMFYALSRYADISGCIALTNAHTKENVLYEEFLKLSCRLAESFKKYGLKQNDTIAVCSENGLQFFLPIIASLYLGIIAAPVSDKYIERELIHSLGIVKPRIIFCSKNTFQKVLNVKSKLKYVETIIILDLNEDLGGYQCLNNFISQNSDINLDVKKFKPYSFNRDDQVALVMFSSGTTGVSKGVMLTHKNIVARFSLAKDPTFGNAINPTTAILTVIPFHHGFGMMTTLGYFTCGFRVVLMHTFEEKLFLQSLQDYKVESTLLVPTLMAFLAKSALVEKYDLSHLKEIASGGAPLSKEIGEMVKKRFKLNFVRQGYGLTETTSAVLITPNNDVRPGSTGKIVPFHAVKVVDPTTGKILGPNEPGELYFKGDMIMKGYYNNEEATKAIINKDGWLRSGDIAYYDNDGHFYIVDRLKSLIKYKGYQVAPAEIEGILLQHPYIVDAGVTGIPDEAAGELPAAGVVVQTGKYLNEQIVQNFVSSQVSTAKWLRGGVKFLDEIPKGSTGKIDRKVLRQMFEKHTNG

SEQ ID NO: 55 - protein, 542 aa, synthetic construct
  REGION 1..542: note = LucPpL-81-6G1
MMKREKNVIYGPEPLHPLEDLTAGEMLFRALRKHSHLPQALVDVVGDESLSYKEFFEATVLLAQSLHNCGYKMNDVVSICAENNTRFFIPVIAAWYIGMIVAPVNESYIPDELCKVMGISKPQIVFTTKNILNKVLEVQSRTNFIKRIIILDTVENIHGCESLPNGISRYSDGNIANFKPLHFDPVEQVAAILCSSGTTGLPKGVMQTHQNICVRLIHALDPRAGTQLIPGVTVLVYLPFFHAFGFSITLGYFMVGLRVIMFRRFDQEAFLKAIQDYEVRSVINVPSVILFLSKSPLVDKYDLSSLRELCCGAAPLAKEVAEVAAKRLNLPGIRCGFGLTESTSANIHSLRDEFKSGSLGRVTPLMAAKIADRETGKALGPNQVGELCIKGPMVSKGYVNNVEATKEAIDDDGWLHSGDFGYYDEDEHFYVVDRYKELIKYKGSQVAPAELEEILLKNPCIRDVAVVGIPDLEAGELPSAFVVKQPGKEITAKEVYDYLAERVSHTKYLRGGVRFVDSIPRNVTGKITRKELLKQLLEKAGG

SEQ ID NO: 56 - protein, 542 aa, synthetic construct
  REGION 1..542: note = CBGRluc
MVKREKNVIYGPEPLHPLEDLTAGEMLFRALRKHSHLPQALVDVVGDESLSYKEFFEATVLLAQSLHNCGYKMNDVVSICAENNTRFFIPVIAAWYIGMIVAPVNESYIPDELCKVMGISKPQIVFTTKNILNKVLEVQSRTNFIKRIIILDTVENIHGCESLPNFISRYSDGNIANFKPLHFDPVEQVAAILCSSGTTGLPKGVMQTHQNICVRLIHALDPRYGTQLIPGVTVLVYLPFFHAFGFHITLGYFMVGLRVIMFRRFDQEAFLKAIQDYEVRSVINVPSVILFLSKSPLVDKYDLSSLRELCCGAAPLAKEVAEVAAKRLNLPGIRCGFGLTESTSAIIQTLGDEFKSGSLGRVTPLMAAKIADRETGKALGPNQVGELCIKGPMVSKGYVNNVEATKEAIDDDGWLHSGDFGYYDEDEHFYVVDRYKELIKYKGSQVAPAELEEILLKNPCIRDVAVVGIPDLEAGELPSAFVVKQPGTEITAKEVYDYLAERVSHTKYLRGGVRFVDSIPRNVTGKITRKELLKQLLVKAGG

SEQ ID NO: 57 - protein, 490 aa, synthetic construct
  REGION 1..490: note = Embryonic Alkaline Phosphatase (EAP)
MIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPG

SEQ ID NO: 58 - protein, 236 aa, synthetic construct
  REGION 1..236: note = mCherry
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 59 - protein, 239 aa, synthetic construct
  REGION 1..239: note = EGFP (Enhanced Green Fluorescent Protein)
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 60 - protein, 174 aa, synthetic construct
  REGION 1..174: note = MYR-GGS-GLuc (M60L/M127L variant)
MGSSKSKPKDPSQRRRGGSGDPKPTENNEDADRGKLPGKKLPLEVLKELEANARKAGCTRGCLICLSHIKCTPKMKKFIPGRCHTYEGDKESAQGGIGEAIVDIPEIPGFKDLEPLEQFIAQVDLCVDCTTGCLKGLANVQCSDLLKKWLPQRCATFASKIQGQVDKIKGAGGD

SEQ ID NO: 61 - DNA, 25 bp, synthetic construct
  misc_feature 1..25: note = FLAG-TAG
  source 1..25: mol_type = other DNA
gattataaagatgatgacgataaag

SEQ ID NO: 62 - DNA, 90 bp, synthetic construct
  misc_feature 1..90: note = 3xFLAG
  source 1..90: mol_type = other DNA
gactacaaggacgacgatgacaaggattataaagatgatgacgataaagtcgagaccgatttctacgactacaaggacgacgatgacaag

SEQ ID NO: 63 - DNA, 27 bp, synthetic construct
  misc_feature 1..27: note = HA-TAG
  source 1..27: mol_type = other DNA
tacccctacgatgtgcccgattacgct

SEQ ID NO: 64 - DNA, 27 bp, synthetic construct
  misc_feature 1..27: note = AcV5-TAG
  source 1..27: mol_type = other DNA
agttggaaggatgcctctggatggtct

SEQ ID NO: 65 - DNA, 30 bp, synthetic construct
  misc_feature 1..30: note = MYC-TAG
  source 1..30: mol_type = other DNA
gagcagaaactgatctcggaagaggatctg

SEQ ID NO: 66 - DNA, 54 bp, synthetic construct
  misc_feature 1..54: note = T2A
  source 1..54: mol_type = other DNA
gagggcagaggaagtctactaacctgcggagatgtggaagaaaatcctggccca

SEQ ID NO: 67 - DNA, 57 bp, synthetic construct
  misc_feature 1..57: note = P2A
  source 1..57: mol_type = other DNA
gcaacaaacttctcactactcaaacaagcaggtgacgtggaggagaatcccgggcct

SEQ ID NO: 68 - DNA, 57 bp, synthetic construct
  misc_feature 1..57: note = P2A-variant
  source 1..57: mol_type = other DNA
gcaacaaacttctcactactcaaacaagcaggtgacgtggagcagaatcccgggcct

SEQ ID NO: 69 - DNA, 60 bp, synthetic construct
  misc_feature 1..60: note = E2A
  source 1..60: mol_type = other DNA
caatgcactaattacgcattgctcaagctggccggtgacgttgagagtaatcctggcccc

SEQ ID NO: 70 - protein, 8 aa, synthetic construct
  REGION 1..8: note = FLAG-TAG
DYKDDDDK

SEQ ID NO: 71 - protein, 30 aa, synthetic construct
  REGION 1..30: note = 3xFLAG
DYKDDDDKDYKDDDDKVETDFYDYKDDDDK

SEQ ID NO: 72 - protein, 9 aa, synthetic construct
  REGION 1..9: note = HA-TAG
YPYDVPDYA

SEQ ID NO: 73 - protein, 9 aa, synthetic construct
  REGION 1..9: note = AcV5-TAG
SWKDASGWS

SEQ ID NO: 74 - protein, 10 aa, synthetic construct
  REGION 1..10: note = MYC-TAG
EQKLISEEDL

SEQ ID NO: 75 - protein, 18 aa, synthetic construct
  REGION 1..18: note = T2A
EGRGSLLTCGDVEENPGP

SEQ ID NO: 76 - protein, 19 aa, synthetic construct
  REGION 1..19: note = P2A
ATNFSLLKQAGDVEENPGP

SEQ ID NO: 77 - protein, 19 aa, synthetic construct
  REGION 1..19: note = P2A-variant
ATNFSLLKQAGDVEQNPGP

SEQ ID NO: 78 - protein, 20 aa, synthetic construct
  REGION 1..20: note = E2A
QCTNYALLKLAGDVESNPGP

SEQ ID NO: 79 - DNA, 2292 bp, synthetic construct
  misc_feature 1..2292: note = FMC63-MYC-BBZ-T2A-EGFP CAR
  source 1..2292: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcacgcgtagagcagaaactgatctcggaagaggatctggcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctgaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctctagtggctccggcgagggcagaggaagtctactaacctgcggagatgtggaagaaaatcctggcccaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 80 - DNA, 2175 bp, synthetic construct
  misc_feature 1..2175: note = FMC63-Myc-BBz-T2A-PAC CAR
  source 1..2175: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcacgcgtagagcagaaactgatctcggaagaggatctggcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctgaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctctagtggctccggcgagggcagaggaagtctactaacctgcggagatgtggaagaaaatcctggcccacatatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 81 - DNA, 2157 bp, synthetic construct
  misc_feature 1..2157: note = FMC63-Myc-28z-T2A-PAC CAR
  source 1..2157: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcacgcgtagagcagaaactgatctcggaagaggatctgaaaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccacgccgccccggacccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctctagtggctccggcgagggcagaggaagtctactaacctgcggagatgtggaagaaaatcctggcccacatatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 82 - DNA, 2199 bp, synthetic construct
  misc_feature 1..2199: note = CD8SP-2-CD19MM-(vL-vH)-Myc-BBz-T2A-PAC CAR
  source 1..2199: mol_type = other DNA
atggccctgcctgtgacagctctgctgctgcctctggcactgctgctgcacgccgccagacctgacatccagctgacacagagccctgccagcctggccgtgtctctgggacagagagccaccatcagctgcaaggccagccagagcgtggactacgacggcgacagctacctgaactggtatcagcagatccccggccagccccccaagctgctgatctacgatgccagcaacctggtgtccggcatcccccctagattttccggcagcggctccggcaccgacttcaccctgaatatccaccccgtggaaaaggtggacgccgccacctaccactgccagcagagcaccgaagatccctggacctttggcggaggcaccaagctggaaatcaagggaggcggaggaagtggcggcggaggatctgggggaggcggaagccaggtacagctccaacagtcaggggccgagctggtacgcccagggtccagcgtcaaaatatcatgtaaggcatctggctacgccttcagctcttactggatgaattgggtgaaacaacggccaggccagggactggagtggattggtcaaatttggccaggagacggggacacaaattacaacggaaaattcaagggaaaggccacacttaccgccgatgaaagtagttcaaccgcttatatgcagctgtcctccctcgcttctgaggactctgctgtgtacttttgcgcacgccgagaaaccaccactgtgggaagatactactacgctatggattattggggacagggcacaaccgtgaccgttagcagtacgcgtgagcagaaactgatctcggaagaggatctggcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctgaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctctagtggctccggcgagggcagaggaagtctactaacctgcggagatgtggaagaaaatcctggcccacatatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 83 - DNA, 2199 bp, synthetic construct
  misc_feature 1..2199: note = 4C3-Myc-BBz-T2A-PAC CAR
  source 1..2199: mol_type = other DNA
atggccttaccagtgaccgccttgctcctgccgctggccttgctgctccacgccgccaggccggacatcgtgatgacccagagccccagcagcctgaccatgagcgtgggccagaaagtgaccatgtcctgcaagagcagccacagcctgctgaacagccggaaccagaagaactacctggcctggttccagcagaagcccggccagtcccccaagctgctggtgtacttcgcaagcacccgcgagagcggcgtgcccgacagattcatcggcagcggctccggcaccgacttcaccctgaccatcagcaacgtgcaggccgaggacctggccgactacttctgccagcagcactacaccaccctgcccaccttcggcggaggcaccaagctggaaatcaagggcggaggcggaagtggaggcggaggatctggcggcggaggctctcagatccagctggtgcagagcggccctgacctgaagaaacccggcgagacagtgaagatcagttgcaaggcctccggctacaccttcaccaacacaggcatgaactgggtcaagcaggcccctggcaagggcctgaagtggatgggctggatcaacacctacaccggcgagcccacctacgccgacgacttcaagggccggttcaccttcagcctggaaaccagcgccagcaccgcctacctgcagatcaacaacctgaagaacgaggacaccgccacctatttctgcaccagatggggctacggcagcagcctgtactacgccatggactactggggccagggcaccagcgtgaccgtgtcctctacgcgtgagcagaaactgatctcggaagaggatctggcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctgaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctctagtggctccggcgagggcagaggaagtctactaacctgcggagatgtggaagaaaatcctggcccacatatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 84 - DNA, 2754 bp, synthetic construct
  misc_feature 1..2754: note = CD8SP-TROP2-h7E6-SVG-(vL-vH)-Myc-z-P2A-K13-Flag-T2A -PAC CAR
  source 1..2754: mol_type = other DNA
atggccttaccagtgaccgccttgctcctgccgctggccttgctgctccacgccgccaggccggacatcgtgatgacccagagccccgatagcctggccgtgtctctgggagagagagccaccatcaactgccgggccagcaagagcgtgtccaccagcggctacagctacatgcactggtatcagcagaagcccggccagccccccaagctgctgatctacctggcctccaacctggaaagcggcgtgcccgatagattcagcggcagcggctctggcaccgacttcaccctgaccatcagttctcttcaggccgaggacgtggccgtgtactactgccagcacagcagagagctgccctacaccttcggccagggcaccaagctggaaatcaagggcggaggcggaagtggaggcggaggatctggcggcggaggctctcaggtgcaactccaggaatctggccctggccttgtgaagcccagcgagacactgagcctgacctgtaccgtgtccggcggcagcatcagctcttacggcgtgcactggattcggcagcctccaggcaagggcctggaatggatcggcgtgatctggacctccggcgtgaccgactacaacagcgccctgatgggcagagtgaccatctccgtggacaccagcaagaaccagttcagcctgaagctgagcagcgtgacagccgccgataccgccgtgtattattgcgccagagatggcgactacgaccgctacacaatggactactggggccagggaacccttgtgaccgtgtcctctacgcgtgagcagaaactgatctcggaagaggatctggcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgacatctacatctgggcgcccttggccgggacttgtggggtccttctcctgtcactggttatcaccctttactgcgggcgcgccagagtgaagttcagcaggagcgcagacgcccccgcgtaccagcagggccagaaccagctctataacgagctgaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctctagaggatctggagcaacaaacttctcactactcaaacaagcaggtgacgtggaggagaatcccgggcctgggatccacatggccacttacgaggttctctgtgaggtggcgcggaaactgggcacggatgacagggaagtggtattgttcctcctaaacgtgttcatacctcaacccacactggcccaattaattggagctcttagagctttaaaggaggagggcaggttaacgtttcccctgttagcggaatgtctgtttcgtgcaggtcgcagagacctcttgcgcgacctgcttcacttagacccgcgttttttagagcgccacctagcgggcacaatgagttatttcagcccttatcagctcactgttctccacgtagacggggagctgtgtgcgagggatattaggtctttgatatttttaagcaaggacactatagggtctcgcagcacaccacagacattcttacactgggtgtactgtatggaaaacttagacctactgggtcccactgacgtggatgccctaatgtcaatgcttagatctttgtcaagagtagacctacagcgccaagtgcaaaccctaatgggccttcacctttccggcccatcacactcccaacactatcgccatacaccactcgagaccgatttctacgactacaaggacgacgatgacaagactagtggctccggcgagggcagaggaagtctactaacctgcggagatgtggaagaaaatcctggcccacatatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 85 - protein, 763 aa, synthetic construct
  REGION 1..763: note = FMC63-MYC-BBZ-T2A-EGFP CAR
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSRVEQKLISEEDLAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSSGSGEGRGSLLTCGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 86 - protein, 724 aa, synthetic construct
  REGION 1..724: note = FMC63-Myc-BBz-T2A-PAC CAR
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSRVEQKLISEEDLAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSSGSGEGRGSLLTCGDVEENPGPHMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 87 - protein, 718 aa, synthetic construct
  REGION 1..718: note = FMC63-Myc-28z-T2A-PAC CAR
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSRVEQKLISEEDLKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSSGSGEGRGSLLTCGDVEENPGPHMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 88 - protein, 732 aa, synthetic construct
  REGION 1..732: note = CD8SP-2-CD19MM-(vL-vH)-Myc-BBz-T2A-PAC CAR
MALPVTALLLPLALLLHAARPDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSTREQKLISEEDLAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSSGSGEGRGSLLTCGDVEENPGPHMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 89 - protein, 732 aa, synthetic construct
  REGION 1..732: note = 4C3-Myc-BBz-T2A-PAC CAR
MALPVTALLLPLALLLHAARPDIVMTQSPSSLTMSVGQKVTMSCKSSHSLLNSRNQKNYLAWFQQKPGQSPKLLVYFASTRESGVPDRFIGSGSGTDFTLTISNVQAEDLADYFCQQHYTTLPTFGGGTKLEIKGGGGSGGGGSGGGGSQIQLVQSGPDLKKPGETVKISCKASGYTFTNTGMNWVKQAPGKGLKWMGWINTYTGEPTYADDFKGRFTFSLETSASTAYLQINNLKNEDTATYFCTRWGYGSSLYYAMDYWGQGTSVTVSSTREQKLISEEDLAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSSGSGEGRGSLLTCGDVEENPGPHMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 90 - protein, 917 aa, synthetic construct
  REGION 1..917: note = CD8SP-TROP2-h7E6-SVG-(vL-vH)-Myc-z-P2A-K13-Flag-T2A -PAC CAR
MALPVTALLLPLALLLHAARPDIVMTQSPDSLAVSLGERATINCRASKSVSTSGYSYMHWYQQKPGQPPKLLIYLASNLESGVPDRFSGSGSGTDFTLTISSLQAEDVAVYYCQHSRELPYTFGQGTKLEIKGGGGSGGGGSGGGGSQVQLQESGPGLVKPSETLSLTCTVSGGSISSYGVHWIRQPPGKGLEWIGVIWTSGVTDYNSALMGRVTISVDTSKNQFSLKLSSVTAADTAVYYCARDGDYDRYTMDYWGQGTLVTVSSTREQKLISEEDLAKPTTTPAPRPPTPAPTIASQPLSLRPEACRPAAGGAVHTRGLDFACDIYIWAPLAGTCGVLLLSLVITLYCGRARVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSRGSGATNFSLLKQAGDVEENPGPGIHMATYEVLCEVARKLGTDDREVVLFLLNVFIPQPTLAQLIGALRALKEEGRLTFPLLAECLFRAGRRDLLRDLLHLDPRFLERHLAGTMSYFSPYQLTVLHVDGELCARDIRSLIFLSKDTIGSRSTPQTFLHWVYCMENLDLLGPTDVDALMSMLRSLSRVDLQRQVQTLMGLHLSGPSHSQHYRHTPLETDFYDYKDDDDKTSGSGEGRGSLLTCGDVEENPGPHMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 91 - DNA, 7488 bp, synthetic construct
  misc_difference 3291..3294: note = n is A, C, G, or T
  source 1..7488: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgataagctttgcaaagatggataaagttttaaacagagaggaatctttgcagctaatggaccttctaggtcttgaaaggagtgcctcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgaccttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcactatagggagacccaagctggctaggtaagcttgatatcgaattcctgcagcccgggggatctgctagcnnnngtcgactccggatgatcagggccctgtacagatatcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 92 - DNA, 7512 bp, synthetic construct
  misc_feature 1..7512: note = pLENTI-NLuc-AcV5-Blasticidin-Pa08
  source 1..7512: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctagaggatccccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccctatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgagttggaaggatgcctctggatggtcttaagtcgagaccgatttctacccctacgatgtgcccgattacgcttagtctagagggcccgcggttcgaaggtaagcctatccctaaccctctcctcggtctcgattctacgcgtaccggttagtaatgagtttggaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 93 - DNA, 7476 bp, synthetic construct
  misc_feature 1..7476: note = pLENTI-TurboLuc-16-X3Flag-Blast-C04
  source 1..7476: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctagaggatccccatggaagccgaggccgagagaggcaagctgcccggcaaaaagctgcccctggaagtgctgatcgagctggaagccaacgccagaaaggccggctgcaccagaggctgcctgatctgcctgagcaagatcaagtgcaccgccaagatgaagaagtacatccccggcagatgcgccgactacggcggcgataagaaaacaggccaggccggcatcgtgggagccatcgtggatatccctgagatcagcggcttcaaagaaatggaacccatggaacagtttatcgcccaggtggaccgctgcgccgattgcacaacaggctgtctgaagggcctggctaacgtgaagtgcagcgacctgctgaagaagtggctgcctggcagatgtgccaccttcgccgacaagatccagagcgaggtggacaacatcaagggactggccggcgacctcgagaccgatttctacgactacaaggacgacgatgacaaggattataaagatgatgacgataaagtcgagaccgatttctacgactacaaggacgacgatgacaagtagtctagagggcccgcggttcgaaggtaagcctatccctaaccctctcctcggtctcgattctacgcgtaccggttagtaatgagtttggaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 94 - DNA, 8663 bp, synthetic construct
  misc_feature 1..8663: note = pLenti-EF1a-Pac-T2A-Gluc-B07
  source 1..8663: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgataagctttgcaaagatggataaagttttaaacagagaggaatctttgcagctaatggaccttctaggtcttgaaaggagtgcctcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgaccttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcactatagggagacccaagctggctagttaagcttgatatcgaattcctgcagcccgggggatctgctagcatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccactagtggctccggcgagggcagaggaagtctactaacctgcggagatgtggaagaaaatcctggcccacatatgaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaactcgactccggatgatcagggccctgtacagatatcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 95 - DNA, 7566 bp, synthetic construct
  misc_feature 1..7566: note = MSCVhygro-GLuc-HA-G02
  source 1..7566: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattagatccccaccatgaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgacctcgagaccgatttctacccctacgatgtgcccgattacgcttagtctagagggccctattctatagtgtcacctaagtcgaggttaacgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcatcccgccaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccggggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgaacaagagctgatttcgagaacgcctcagccagcaactcgcgcgagcctagcaaggcaaatgcgagagaacggccttacgcttggtggcacagttctcgtccacagttcgctaagctcgctcggctgggtcgcgggagggccggtcgcagtgattcaggcccttctggattgtgttggtccccagggcacgattgtcatgcccacgcactcgggtgatctgactgatcccgcagattggagatcgccgcccgtgcctgccgattgggtgcagatccgtcgacctgcagccaagcttatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaagagctggggagaattaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaaggggggaa

SEQ ID NO: 96 - DNA, 6884 bp, synthetic construct
  misc_feature 1..6884: note = MSCVpac-GLUC-HA-R03
  source 1..6884: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattagatccccaccatgaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgacctcgagaccgatttctacccctacgatgtgcccgattacgcttagtctagagggccctattctatagtgtcacctaagtcgaggttaacgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaagagctggggagaattaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaaggggggaa

SEQ ID NO: 97 - DNA, 7188 bp, synthetic construct
  misc_feature 1..7188: note = MSCVneo-Lucia-Luc-x3-Flag-B08
  source 1..7188: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattagatccccaccatgaaacccactgaaatcaatgaagacctcaatatagctgctgtggcctccaactttgccaccacagatcttgagactgacctgttcaccaactgggagaccatgaatgtgattagcactgacacagagcaggtgaacacagatgctgacaggggcaagctgcctggcaaaaaactccccccagatgtcctgagggagctggaggccaatgccagaagggctggttgcacaagaggctgcctcatttgcctctcccacattaagtgcacccctaagatgaagaaatttatccctggcaggtgccacacttatgaaggtgaaaaggagtctgctcagggagggattggagaggcaattgttgatatcccagagattcctggcttcaaggataaggagccactggaccagtttattgctcaagtggacctctgtgctgattgcaccactggctgtctgaagggccttgccaatgtccagtgctctgacctcctgaagaagtggcttccccagaggtgtaccacttttgccagcaagattcagggtagggtggacaaaatcaagggtctggctggggacagactcgagaccgatttctacgactacaaggacgacgatgacaaggattataaagatgatgacgataaagtcgagaccgatttctacgactacaaggacgacgatgacaagtagtctagagggccctattctatagtgtcacctaagtcgacgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgtcgacctgcagccaagcttatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaagagctggggagaattaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaaggggggaa

SEQ ID NO: 98 - DNA, 7476 bp, synthetic construct
  misc_feature 1..7476: note = pLENTI-Gluc-Flag-blast-B07
  source 1..7476: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctagaggatccccaccatgaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgacctcgagaccgatttctacgactacaaggacgacgatgacaagtagtctagagggcccgcggttcgaaggtaagcctatccctaaccctctcctcggtctcgattctacgcgtaccggttagtaatgagtttggaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 99 - DNA, 9294 bp, synthetic construct
  misc_feature 1..9294: note = pLENTI-EF1a-fFLuc-Blasticidin-A07
  source 1..9294: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgataagctttgcaaagatggataaagttttaaacagagaggaatctttgcagctaatggaccttctaggtcttgaaaggagtgcctcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgaccttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcactatagggagacccaagctggctagttaagcttgatatcgaattcctgcagcccgggggatctgctagtccagtgtggtggaattcgaagctcgagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaaccggttagtaatgagtttggaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 100 - DNA, 9839 bp, synthetic construct
  misc_feature 1..9839: note = pLenti-EF1-LucPPe-146-1H2-Flag-Pac-R01
  source 1..9839: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgataagctttgcaaagatggataaagttttaaacagagaggaatctttgcagctaatggaccttctaggtcttgaaaggagtgcctcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgaccttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcactatagggagacccaagctggctaggtaagcttgatatcgaattcctgcagcccgggggatctgctagcggatccaccatggccgacaagaacatcctgtacggccccgagcctttctaccctctggaagatggaacagccggcgagcagatgttcgacgccctgagcagatatgccgccattcctggatgtatcgccctgacaaacgcccacaccaaagaaaacgtgctgtacgaagagttcctgaagctgagctgtcggctggccgagagcttcaagaagtacggcctgaagcagaacgacacaatcgccgtgtgcagcgagaacagcctccagttcttcctgcctgtgatcgccagcctgtacctgggcattattgtggcccctgtgaacgacaagtacatcgagagagagctgatccacagcctgggcatcgtgaagccccggatcgtgttctgctccaagaacaccttccagaaggtgctgaacgtcaagagcaagctgaagtccatcgagacaatcatcatcctggacctgaacgaggacctcggcggctaccagtgcctgaacaacttcatcagccagaacagcgacagcaacctggacgtgaagaagttcaagccctacagcttcaaccgggacgaccaggtggcctccatcatgtttagcagcggcaccaccggactgcccaaaggcgttatgctgacccacaagaatatcgtggcccggttctctatcgctaaggaccccaccttcggcaacgccatcaatcctacaagcgctatcctgacagtgatccccttccaccacggcttcggcatgatgaccacactgggctacttcacctgtggcttcagagtggtgctgatgcacaccttcgaggaaaagctgtttctccagagcctccaggactacaaggtggaaagcaccctgctggtgcctactctgatggccttcctggctaagtctgccctggtcgagaagtacgatctgagccacctgaaagagatcgcctctggcggagcccctctgagcaaagaaatcggcgagatggtcaagaagcggttcaagctgaacttcgtgcggcaaggctatggcctgaccgagacaacaagcgccgtgctgattacccctaagggcgacgccaagcctggcagcacaggcaaaattgtgcctctgcacgccgtgaaggtggtggaccctaccacaggcaagatcctgggacctaatgagcccggcgagctgtacttcaagggacccatgattatgaagggctactacaacaacgaggaagccaccaaggccattatcgacaacgacggctggctgcggagcggcgatattgcctactacgacaatgacggccacttctacatcgtggacagactgaagtccctcatcaagtacaagggctatcaggtggccccagccgagatcgagggtatcctgctgcaacacccctatatcgtggatgccggcgtgacaggcatccctgatgaagctgctggcgaacttccagcagctggcgtggtggttcagaccggcaagtacctgaatgagcagatcgtgcaggactacgtggcctctcaggtgtccacagccaaatggctgagaggcggcgtgaagttcctggacgagatcccaaagggctctaccggcaagatcgacagaaaggtgctgcggcagatgctggaaaagcacaccaatggcctcgagaccgatttctacgactacaaggacgacgatgacaagactagtggctccggcgagggcagaggaagtctactaacctgcggagatgtggaagaaaatcctggcccacatatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagtcgactccggatgatcagggccctgtacagatatcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 101 - protein, 26 aa, synthetic construct
  VARIANT 2..4: note = Xa can be any amino acid.
  VARIANT 7..8: note = Xa can be any amino acid.
  VARIANT 10..13: note = Xa can be any amino acid.
  VARIANT 15..22: note = Xa can be any amino acid.
  VARIANT 24: note = Xa can be any amino acid.
CXXXCLXXLXXXXCXXXXXXXXPXRC

SEQ ID NO: 102 - protein, 56 aa, synthetic construct
  VARIANT 2..8: note = X can be any amino acid
  VARIANT 15..17: note = X can be any amino acid
  VARIANT 20: note = X can be any amino acid
  VARIANT 22: note = X can be any amino acid
  VARIANT 26: note = X can be any amino acid
  VARIANT 29: note = X can be any amino acid
  VARIANT 34: note = X can be any amino acid
  VARIANT 37..40: note = X can be any amino acid
  VARIANT 46: note = X can be any amino acid
  VARIANT 48..55: note = X can be any amino acid
KXXXXXXXEMEANAXXXGCXRXCLIXLSXIKCTXKMXXXXPGRCHXYXXXXXXXXG

SEQ ID NO: 103 - protein, 64 aa, synthetic construct
  VARIANT 2: note = X can be any amino acid
  VARIANT 4..9: note = X can be any amino acid
  VARIANT 11: note = X can be any amino acid
  VARIANT 14..15: note = X can be any amino acid
  VARIANT 18..19: note = X can be any amino acid
  VARIANT 21..22: note = X can be any amino acid
  VARIANT 24..26: note = X can be any amino acid
  VARIANT 34..35: note = X can be any amino acid
  VARIANT 38..39: note = X can be any amino acid
  VARIANT 41..43: note = X can be any amino acid
  VARIANT 46: note = X can be any amino acid
  VARIANT 49..50: note = X can be any amino acid
  VARIANT 52..54: note = X can be any amino acid
  VARIANT 56..63: note = X can be any amino acid
IXGXXXXXXMXQFXXQVXXCXXCXXXCLKGLANXXCSXXLXXXLPXRCXXFXXXIXXXXXXXXG

(19) United States
(12) Patent Application Publication
(10) Pub. No.: US 2025/0327807 A1
(43) Pub. Date: Oct. 23, 2025

(54) NON-RADIOACTIVE CYTOTOXICITY ASSAYS

(71) Applicants: University of Southern California, Los Angeles, CA (US); Angeles Therapeutics, inc., Toluca Lake, CA (US)

(72) Inventor: Preet M Chaudhary, Toluca Lake, CA (US)

(21) Appl. No.: 18/983,951

(22) Filed: Dec. 17, 2024

Related U.S. Application Data
(63) Continuation of application No. 17/723,284, filed on Apr. 18, 2022, now Pat. No. 12,196,759.

(51) Int. Cl. G01N 33/58; C12Q 1/44; C12Q 1/66; G01N 33/50

(52) U.S. Cl. CPC G01N 33/581; C12Q 1/44; C12Q 1/66; G01N 33/5014; G01N 33/5017; G01N 33/502; G01N 33/582

(57) ABSTRACT

Described herein are methods for assessing cytotoxicity of an agent. The methods include providing a target cell that has been engineered to express intracellularly a reporter that is not expressed endogenously by the target cell, exposing the target cell to an agent capable of modulating cytotoxicity and assaying the activity of the reporter, wherein a change in reporter activity relative to a reference value is indicative of the agent being able to modulate cytotoxicity of the target cell.

### CROSS-REFERENCE TO RELATED APPLICATION

This application is a continuation of U.S. application Ser. No. 17/723,284, filed Apr. 18, 2022, which is a continuation of U.S. application Ser. No. 16/333,980, filed Mar. 15, 2019, which application claims priority as a U.S. National Stage Application filed under 35 U.S.C. § 371 to International Application No. PCT/US2017/052344, filed Sep. 19, 2017, which application claims the benefit of priority under 35 U.S.C. § 119(e) of U.S. Provisional Patent Application No. 62/396,650, filed on Sep. 19, 2016, the contents of which are incorporated herein by reference in their entirety.

### STATEMENT REGARDING FEDERALLY SPONSORED RESEARCH OR DEVELOPMENT

This invention was made with government support under Grant No. DE019811 and DE025804 awarded by National Institutes of Health. The government has certain rights in the invention.

### INCORPORATION BY REFERENCE OF SEQUENCE LISTING

Accompanying this filing is a Sequence Listing entitled “18983951_07-14-2025_ANGE_100242.xml” with a creation date of Jul. 14, 2025 and having 272,677 bytes of data, machine formatted on IBM-PC, MS-Windows operating system using the WIPO Sequence (version release date Jun. 10, 2025) software application. The sequence listing is hereby incorporated herein by reference in its entirety for all purposes.

### FIELD OF THE INVENTION

Provided herein are non-radioactive assays to assess cytotoxicity of a compound or a therapeutic agent.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Current rational for development of targeted therapeutic approaches largely relies on development of agents that selectively induce cytotoxicity and eliminate diseased cells associated with pathological conditions including cancer. These agents range from small molecules or biologics such as peptides, antibodies or shRNAs or T cells genetically engineered to selectively recognize tumor antigens. Successful selection and optimization of these agents for downstream application in immune-oncology, adoptive cellular therapy, CAR and antibody therapy depends on the accuracy and sensitivity of assays employed to measure cytotoxicity. In addition to drug discovery, methods to determine cell viability or cytotoxicity in response to exposure to a given test agent are also key to environmental testing and pesticide and herbicide testing. In short, to determine whether a given agent presents a real or potential risk when exposed to a given cell type requires a method that reliably, precisely, and accurately measures cell toxicity and/or viability after exposure to the test agent.

Several assays have been developed to measure cytotoxicity. Of these, radioactive chromium (Cr⁵¹) release assay developed in 1968 is most commonly used worldwide. In this assay, target cells labeled with Cr⁵¹ are incubated with effector cells and Cr⁵¹ released upon their lysis serves as a measure of the effector cell cytotoxicity. However, several limitations including the hazards associated with harmful effects of radioactivity, additional costs of disposal of radioactive waste and requirement of additional equipment like gamma counters, have prompted researchers to seek safer alternative approaches. For example, cell membranes of target cells can be labeled with fluorescent dyes and cytotoxic response can be evaluated using multicolor flow cytometric analysis. However, the successful application of this approach demands careful calibration and labor intensive data analysis to efficiently distinguish the target and effector cell populations.

Non-viable cells that have lost membrane integrity leak cytoplasmic components into the surrounding medium. Cell death can be measured by monitoring the concentration of these leaked cellular components in the surrounding medium. Some cytotoxicity assays are based on quantification of the release of enzymes such as lactose dehydrogenase (LDH), glyceraldehyde 3-phosphate dehydrogenase (G3PDH) or adenylate kinase (AK) from dead cells. All these assays measure enzyme activity either directly by providing substrates that would be converted to fluorescent or luminescent products or include a second step wherein products of the primary reaction indirectly generate substrate for a luciferase reaction. Most of these enzymatic methods require a two-step procedure to remove culture medium to a separate container and thus are non-homogeneous. Additionally, these methods, in general, have poor sensitivity and, importantly, are unable to distinguish between death of target and effector cells, since both types of cells release cellular enzymes upon lysis.

Bioluminescence is inherited endogenously across a variety of species including bacteria, insects, fungi, and marine organisms. It happens when an enzyme, called a luciferase, oxidizes a light-emitting substrate (e.g. luciferin or coelenterazine). Because of their ability to provide highly sensitive quantitation with broad linearity, luciferases have been used extensively as reporters (Thorne, Inglese et al. 2010). Beetle luciferases form a distinct class with unique structure, evolutionary history and chemical mechanism. Firefly luciferases form a distinct subgroup of Beetle luciferases. Historically, the term “firefly luciferase” or FLuc or FfLuc referred to the enzyme LucPpy from a single species of Photinus pyralis. A luciferase release-based cytotoxicity assay was first described by Schafer et al in 1997 using Flue (Schafer, Schafer et al. 1997). However, the shorter half-life of Flue (<30 minutes) in tissue culture medium hindered its wider use (Thompson, Hayes et al. 1991, Schafer, Schafer et al. 1997). Fu et al also tried to develop a T cell cytotoxicity assay based on measurement of Flue that has been released in the medium of Fluc-expressing target cells upon incubation with cytotoxic T cells (Fu, Tao et al. 2010). Surprisingly, incubation of cytotoxic T cells with the tumor cell targets did not result in significant release of luciferase in the culture medium (Fu, Tao et al. 2010). Therefore, these investigators abandoned the idea of measuring release of luciferase in the supernatant as an assay for cytotoxicity (Schafer, Schafer et al. 1997, Fu, Tao et al. 2010).

A number of novel luciferases have been discovered from deep sea marine organisms. These marine luciferases are smaller in size, ATP-independent and have much brighter luminescence as compared to Flue (Takenaka, Yamaguchi et al. 2012). All of them have an N-terminal 17-22 amino acid consensus sequence that signals secretion (Takenaka, Yamaguchi et al. 2012). In addition to marine luciferase and their engineered-derivatives, thermostable variants of luciferases from Photuris pennsylvanica and Pyrophorus plagiothalamus have been described (PCT/US99/30925). Finally, thermostable variants of alkaline phosphatase are known in the art.

In view of the number of therapeutic antibody and cellular products that are now on the market or in development, there is a need for in vitro assays to allow the cytotoxic activity of antibodies and cellular products to be determined. In particular, there is a need for a high-throughput, label-free cytotoxicity assay that is sensitive, inexpensive, quick, homogenous (i.e. can be performed in one step) and also distinguishes between the death of target and effector cells.

### SUMMARY OF THE INVENTION

The following embodiments and aspects thereof are described and illustrated in conjunction with systems, compositions and methods which are meant to be exemplary and illustrative, not limiting in scope.

The invention is directed to a class of assays, termed Matador assays, for determining the cytotoxic effect of a given test compound (including but not limited to chemotherapy agents, antibodies, biologicals, cytotoxic cell (e.g. T cell, NK cell), or a given set of test conditions. The method includes expression of a reporter in target cells in a manner so that it is preferentially retained within the healthy cells but is either released from dead and dying cells or whose activity can be preferentially measured in dead and dying cells. In one embodiment, the inventive method measures the activity of the reporter that has been released from the dead and dying cells. In some embodiments, the reporters are any one or more of: 1) non-secreted forms of luciferases from the copepods, such as Gaussia princeps, Pleuromamma abdominalis, Metridia pacifica, Metridia curticauda, Metridia asymmetrica, Metridia okhotensis, Metridia longa, Lucicutia ovaliformis, Heterorhabdus tanneri, Pleuromamma scutullata or their homologs; 2) engineered luciferase reporters from deep sea shrimp, such as NanoLuc; 3) Renilla luciferase; 4) beetle luciferases, including firefly luciferases and engineered variants such as LucPPe-146-1H2. Other reporters, such as Green Fluorescent Protein, mCherry, and heat-stable alkaline phosphatase may also be used with the assays described herein. However, any molecule (e.g. DNA, RNA or protein) that is not expressed naturally in the target cells, is retained inside the healthy cells but is released from dead and dying cells, can be used. The preferred reporter is stable in the cell culture medium and is not degraded during the conditions of the cytotoxicity assay. As most cytotoxicity assays are carried out at 37° C., the preferred reporter for the assay is also thermostable. Vectors for expressing the reporter genes in stable and transient fashion, engineered cell lines stably expressing the reporter genes, and kits for practicing the invention are also disclosed. In some embodiments, the methods described herein measure the activity of the reporter inside dead and dying cells by addition of a substrate or a cofactor that is required for the activity of the reporter and which is excluded from the healthy cells but preferentially enters dead and dying cells.

Provided herein are methods for assessing cytotoxicity of an agent. The methods include providing a target cell that has been engineered to express intracellularly a reporter; exposing the target cell to an agent capable of modulating cytotoxicity; and assaying the activity of the reporter. In one embodiment, the reporter is not expressed endogenously by the target cell. In another embodiment, the reporter is expressed endogenously by the target cell at a level lower than what is achieved by engineered expression. In one embodiment, a change in reporter activity relative to a reference value is indicative of the agent being able to modulate cytotoxicity of the target cell. In one embodiment, a change in reporter activity is an increase in reporter activity relative to a reference value. In some embodiments, the reporter is expressed endogenously by the target cell at a level that is at least 10-25%, 25-50%, 50-75% or 75-100% lower than what is achieved by engineered expression.

In on embodiment, the reporter activity is assayed in the cell media containing the target cells. In another embodiment, the reporter activity is assayed in the cell supernatant that is free of the target cells.

In various embodiments, the cytotoxicity of an agent is measured by obtaining (assaying) the reporter activity in cell pellet and cell free supernatant and normalizing the reporter activity measured in the cell free supernatant. In various embodiments, normalizing comprises dividing the activity in the cell free supernatant by the reporter activity measured in the cell pellet

In various embodiments, the reporter is a non-secretory form of an enzyme that is stable under the assay conditions at 37° C. for more than 15 min, for more than 30 min, for more than 1 hour, for more than 2 hours, for more than 3 hours, for more than 4 hours, for more than 12 hours, 24 hours, for more than 36 hours, for more than 48 hours or for at least 96 hours.

In various embodiments, the reporter is a non-secretory form of a luciferase. In some embodiments, the non-secretory form of luciferase is obtained from copepods, deep sea shrimp, beetle, firefly, or homologs or orthologs thereof or mutants or variants or derivatives thereof.

In exemplary embodiments, the copepods are selected from the group consisting of any one or more of Gaussia princeps, Pleuromamma abdominalis, Metridia pacifica, Metridia curticauda, Metridia asymmetrica, Metridia okhotensis, Metridia longa, Lucicutia ovaliformis, Heterorhabdus tanneri, and Pleuromamma scutullata.

In various embodiments, the luciferase is any one or more of GLuc, NLuc, MLuc7, HTLuc, PaLuc1, PaLuc2, MpLuc1, McLuc1, MaLuc1, MoLuc1, MoLuc2, MLuc39, PsLuc1, LoLuc1-3, HtLuc2, TurboLuc16 (TLuc), Lucia Luc, Renilla Luc, Firefly luciferase (FfLuc or Fluc), LucPPe-146-1H2, LucPPe-133-1B2, LucPPe-78-0B10, LucPPe49-7C6A, LucPpL-81-6G1 or CBGRluc or homologs or orthologs or mutants or variants or derivatives thereof.

In various embodiments, the reporter is a non-secretory form of a luciferase obtained from copepods, deep sea shrimp, beetle, firefly or homologs or orthologs thereof or mutant or derivatives thereof and the reporter activity is assayed by exposing the target cells to a luciferase specific substrate.

In various embodiments, the luciferase-specific substrate is coelentrazine or an imidazopyrazinone substrate (furimazine) or a derivative thereof.

In one embodiment, the luciferase-specific substrate is D-luciferin or a derivative thereof.

In one embodiment, the reporter is a thermostable luciferase. In one embodiment, the reporter is a thermostable beetle luciferase. In an embodiment, the thermostable beetle luciferase is obtained from Photuris pennsylvanica and Pyrophorus plagiothalamus

In some embodiments, the reporter is a non-secretory form of an alkaline phosphatase. In one embodiment, the alkaline phosphatase is a heat-stable alkaline phosphtase.

In some embodiments, the reporter is a non-secretory form of a fluorescent protein. In one embodiment, the non-secretory form of a fluorescent protein is green fluorescent protein.

In some embodiments, the non-secretory form of a fluorescent protein is mCherry protein.

In one embodiment, the reporter is a DNA sequence that is not present in the target cells endogenously. In one embodiment, the reporter is an RNA sequence that is not present in the target cells endogenously.

In one embodiment, the target cells express a single type of reporter. In another embodiment, the target cells express more than one type of reporter. In various embodiments, the activity of the two reporters can be measured independent of each other either simultaneously or sequentially.

In some embodiments, the substrate for one of the reporters is coelentrazine or an imidazopyrazinone substrate (e.g., furimazine) or a derivative thereof and the substrate for the other reporter is D-luciferin or a derivative thereof.

In some embodiments, the substrate for one of the reporters is coelentrazine or an imidazopyrazinone substrate (e.g., furimazine) or a derivative thereof and the substrate for the other reporter is pNNP or a derivative thereof.

In some embodiments, the target cells expressing two or more reporters are mixed together prior to the assay.

In some embodiments, the target cells express the non-secretory form of reporter as a fusion protein with one or more of chitin binding protein (CBP), glutathione-S-transferase (GST), polyhistidine (His) tag, FLAG tag, HA tag, Myc tag, V5 tag, AcV5 tag, Myristoylation (Myr) tag or a combination thereof.

In some embodiments, the reference value is the reporter activity in any one or more of (i) target cells that do not express reporter, (ii) target cells that express reporter but are not treated with the test agent(s); (iii) the target cells that are left untreated; (iv) target cells that are not treated with the substrate for the reporter, or (iii) a combination thereof.

In some embodiments, the agent capable of modulating cytotoxicity is any one or more of an antibody, small molecule, chemical compound, radiation agent, cytotoxic cells, biologics or combinations thereof.

In some embodiments, the cytotoxic cells are any one or more of T cells, NK cells, PBMCs or combinations thereof. In some embodiments, the cytotoxic cells are modified to express a chimeric or synthetic receptor or a T cell receptor.

In some embodiments, the antibodies are any one or more of chimeric antibodies, human antibodies, humanized antibodies, bispecific antibodies, bispecific T cell engager, DART, antibody drug conjugates or combination thereof.

In exemplary embodiments, the agent capable of modulating cytotoxicity targets one or more of the antigens selected from a group consisting of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRviii); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); FmsLike Tyrosine Kinase 3 (FLT3); Tumor-associated glycoprotein 72 (TAG72); CD38; CD44v6; a glycosylated CD43 epitope expersed on acute leukemia or lymphoma but not on hematopoietic progenitors, a glycosylated CD43 epitope expressed on non-hematopoietic cancers, Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-llRa); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAlX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDClalp(1-4)bDGlcp(1-1)Cer); transglutaminase 5 (TGS5); high molecular weight-melanoma associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCT A-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B 1 (CYP1B 1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1), MPL, Biotin, c-MYC epitope Tag, CD34, LAMP1 TROP2, GFRalpha4, CDH17, CDH6, NYBR1, CDH19, CD200R, Slea (CA19.9; Sialyl Lewis Antigen) Fucosyl-GM1, PTK7, gpNMB, CDH1-CD324, DLL3, CD276/B7H3, IL11Ra, IL13Ra2, CD179b-IGLll, ALK TCRgamma-delta, NKG2D, CD32 (FCGR2A), Tn ag, CSPG4-HMW-MAA, Tim1-/HVCR1, CSF2RA (GM-CSFR-alpha), TGFbetaR2, VEGFR2/KDR, Lews Ag, TCR-beta1 chain, TCR-beta2 chain, TCR-gamma chain, TCR-delta chain, FITC, Leutenizing hormone receptor (LHR), Follicle stimulating hormone receptor (FSHR), Chorionic Gonadotropin Hormone receptor (CGHR), CCR4, GD3, SLAMF6, SLAMF4, HIV1 envelope glycoprotein, HTLV1-Tax, CMV pp65, EBV-EBNA3c, influenza A hemagglutinin (HA), GAD, PDL1, Guanylyl cyclase C (GCC), KSHV-K8.1 protein, KSHV-gH protein, HLA, HLA-A, HLA-A2, HLA-B, HLA-C, HLA-DP, HLA-DM, HLA-DOA, HLA-DOB, HLA-DQ, HLA-DR or HLA-G.

In some embodiments, the reporter is expressed in cells by any one or more of plasmid vector, adenoviral vector, adenoassociated viral vector, sleeping beauty transposon, piggyback transposon, pCMV (cytomegalovirus) vectors, vaccinia virus vectors, retroviral vectors, lentiviral vectors, SV40 virus vectors, transfection of naked DNA or transfection of in vitro transcribed RNA. In some embodiments, the reporter is expressed using a non-vector method. In some embodiments, the reporter is expressed from a foreign promoter. In some embodiments, the reporter is expressed from a natural promoter.

In some embodiments, the target cells are exposed to the test agent in vitro. In some embodiments, the target cells are exposed to the test agent in vivo.

In some embodiments, the assay is performed in vitro. In some embodiments, the assay is performed in a high throughput fashion. In some embodiments, the assay is performed in vivo. In some embodiments, the target cells are present in a transgenic animal. In some embodiments, the assay is performed to identify agents that increase, decrease or have no effect on the cytotoxicity of an agent on a target cell. In some embodiments, the assay is performed with one or more agents used alone or in combination.

In some embodiments, the non-secretory form of the reporter is expressed using vectors encoding the non-secretory forms of GLuc, NLuc, MLuc7, HTLuc, PaLuc1, PaLuc2, MpLuc1, McLuc1, MaLuc1, MoLuc1, MoLuc2, MLuc39, PsLuc1, LoLuc1-3, HtLuc2, TurboLuc16 (TLuc), Lucia Luc, Renilla Luc, Firefly luciferase (FfLuc or Fluc), LucPPe-146-1H2, LucPPe-133-1B2, LucPPe-78-0B10, LucPPe49-7C6A, LucPpL-81-6G1 or CBGRluc or homologs or orthologs or mutants or derivatives thereof.

In some embodiments, the target cell is a cell line expressing the non-secretory forms of GLuc, NLuc, MLuc7, HTLuc, PaLuc1, PaLuc2, MpLuc1, McLuc1, MaLuc1, MoLuc1, MoLuc2, MLuc39, PsLuc1, LoLuc1-3, HtLuc2, TurboLuc16 (TLuc), Renilla Luc, Firefly luciferase (FfLuc or Fluc), LucPPe-146-1H2, LucPPe-133-1B2, LucPPe-78-0B10, LucPPe49-7C6A, LucPpL-81-6G1, CBGRluc, thermostable alkaline phosphatase or homologs or orthologs or mutants or derivatives or variant thereof.

In some embodiments, the target cell is a primary cell expressing the non-secretory forms of GLuc, NLuc, MLuc7, HTLuc, PaLuc1, PaLuc2, MpLuc1, McLuc1, MaLuc1, MoLuc1, MoLuc2, MLuc39, PsLuc1, LoLuc1-3, HtLuc2, TurboLuc16 (TLuc), Renilla Luc, Firefly luciferase (FfLuc or Fluc), LucPPe-146-1H2, LucPPe-133-1B2, LucPPe-78-0B10, LucPPe49-7C6A, LucPpL-81-6G1 or CBGRluc or homologs or orthologs or mutants or derivatives thereof.

In some embodiments, the target cell of expresses one or more of the antigens selected from a group consisting of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRviii); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); FmsLike Tyrosine Kinase 3 (FLT3); Tumor-associated glycoprotein 72 (TAG72); CD38; CD44v6; a glycosylated CD43 epitope expersed on acute leukemia or lymphoma but not on hematopoietic progenitors, a glycosylated CD43 epitope expressed on non-hematopoietic cancers, Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-llRa); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAlX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDClalp(1-4)bDGlcp(1-1)Cer); transglutaminase 5 (TGS5); high molecular weight-melanoma associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCT A-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B 1 (CYP1B 1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1), MPL, Biotin, c-MYC epitope Tag, CD34, LAMP1 TROP2, GFRalpha4, CDH17, CDH6, NYBR1, CDH19, CD200R, Slea (CA19.9; Sialyl Lewis Antigen) Fucosyl-GM1, PTK7, gpNMB, CDH1-CD324, DLL3, CD276/B7H3, IL11Ra, IL13Ra2, CD179b-IGLll, ALK TCRgamma-delta, NKG2D, CD32 (FCGR2A), Tn ag, CSPG4-HMW-MAA, Tim1-/HVCR1, CSF2RA (GM-CSFR-alpha), TGFbetaR2, VEGFR2/KDR, Lews Ag, TCR-beta1 chain, TCR-beta2 chain, TCR-gamma chain, TCR-delta chain, FITC, Leutenizing hormone receptor (LHR), Follicle stimulating hormone receptor (FSHR), Chorionic Gonadotropin Hormone receptor (CGHR), CCR4, GD3, SLAMF6, SLAMF4, HIV1 envelope glycoprotein, HTLV1-Tax, CMV pp65, EBV-EBNA3c, influenza A hemagglutinin (HA), GAD, PDL1, Guanylyl cyclase C (GCC), KSHV-K8.1 protein, KSHV-gH protein, HLA, HLA-A, HLA-A2, HLA-B, HLA-C, HLA-DP, HLA-DM, HLA-DOA, HLA-DOB, HLA-DQ, HLA-DR or HLA-G.

In some embodiments, the target cell has been modified and/or selected to lack the expression of one or more antigens. In exemplary embodiments, the target cell has been modified and/or selected to lack the expression of one or more antigens selected from a group consisting of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRviii); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); FmsLike Tyrosine Kinase 3 (FLT3); Tumor-associated glycoprotein 72 (TAG72); CD38; CD44v6; a glycosylated CD43 epitope expersed on acute leukemia or lymphoma but not on hematopoietic progenitors, a glycosylated CD43 epitope expressed on non-hematopoietic cancers, Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-llRa); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAlX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDClalp(1-4)bDGlcp(1-1)Cer); transglutaminase 5 (TGS5); high molecular weight-melanoma associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCT A-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B 1 (CYP1B 1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1), MPL, Biotin, c-MYC epitope Tag, CD34, LAMP1 TROP2, GFRalpha4, CDH17, CDH6, NYBR1, CDH19, CD200R, Slea (CA19.9; Sialyl Lewis Antigen) Fucosyl-GM1, PTK7, gpNMB, CDH1-CD324, DLL3, CD276/B7H3, IL11Ra, IL13Ra2, CD179b-IGLll, ALK TCRgamma-delta, NKG2D, CD32 (FCGR2A), Tn ag, CSPG4-HMW-MAA, Tim1-/HVCR1, CSF2RA (GM-CSFR-alpha), TGFbetaR2, VEGFR2/KDR, Lews Ag, TCR-beta1 chain, TCR-beta2 chain, TCR-gamma chain, TCR-delta chain, FITC, Leutenizing hormone receptor (LHR), Follicle stimulating hormone receptor (FSHR), Chorionic Gonadotropin Hormone receptor (CGHR), CCR4, GD3, SLAMF6, SLAMF4, HIV1 envelope glycoprotein, HTLV1-Tax, CMV pp65, EBV-EBNA3c, influenza A hemagglutinin (HA), GAD, PDL1, Guanylyl cyclase C (GCC), KSHV-K8.1 protein, KSHV-gH protein, HLA, HLA-A, HLA-A2, HLA-B, HLA-C, HLA-DP, HLA-DM, HLA-DOA, HLA-DOB, HLA-DQ, HLA-DR and HLA-G.

In some embodiments, the target cells is derived from a disease selected from the group consisting of a proliferative disease, a precancerous condition, a cancer, an infectious disease, an immune disease, an allergic disease or a degenerative disease.

In some embodiments, the target cell is derived from a cancer chosen from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, primary effusion lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia, colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, Merkel cell cancer, epidermoid cancer, squamous cell cancer, T-cell lymphoma, environmentally induced cancers, combinations of said cancers, and metastatic lesions of said cancers.

In some embodiments, the target cell is associated with infection by a virus including but not limited to HIV1, HIV2, HTLV1, Epstein Barr virus (EBV), cytomegalovirus (CMV), adenovirus, adeno-associated virus, BK virus, Human Herpesvirus 6, Human Herpesvirus 8 influenza virus, parainfluenza virus, avian flu virus, MERS and SARS coronaviruses, Crimean Congo Hemorrhagic fever virus, rhino virus, enterovirus, Dengue virus, West Nile virus, Ebola virus, Marburg virus, Lassa fever virus, zika virus, RSV, measles virus, mumps virus, rhino virus, varicella virus, herpes simplex virus 1 and 2, varicella zoster virus, HIV-1, HTLV1, Hepatitis virus, enterovirus, hepatitis B virus, Hepatitis C virus, Nipah and Rift valley fever viruses, Japanese encephalitis virus, Merkel cell polyomavirus, or is associated with infection with a bacteria, Mycobacterium tuberculosis, atypical mycobacteria species, Pneumocystis jirovecii, toxoplasmosis, rickettsia, nocardia, aspergillus, mucor, or candida.

In some embodiments, the target cell is exposed to an agent capable of modulating cytotoxicity for less than 1 second or for more than 1 second, 30 seconds, 1 minute, 30 minutes, 1 hour, 4 hours, 12 hours, 24 hours, 48 hours, 72 hours, 96 hours, 7 days, or 12 days.

Further provided herein are vectors and/or compositions comprising vectors wherein the vectors encode non-secretory forms of GLuc, NLuc, MLuc7, HTLuc, PaLuc1, PaLuc2, MpLuc1, McLuc1, MaLuc1, MoLuc1, MoLuc2, MLuc39, PsLuc1, LoLuc1-3, HtLuc2, TurboLuc16 (TLuc), Lucia Luc, Renilla Luc, Firefly luciferase (FfLuc or Fluc), LucPPe-146-1H2, LucPPe-133-1B2, LucPPe-78-0B10, LucPPe49-7C6A, LucPpL-81-6G1 or CBGRluc or homologs or orthologs or mutants or derivatives thereof.

Also provided herein are cell lines and/or compositions comprising cell lines wherein the cell lines express the non-secretory forms of GLuc, NLuc, MLuc7, HTLuc, PaLuc1, PaLuc2, MpLuc1, McLuc1, MaLuc1, MoLuc1, MoLuc2, MLuc39, PsLuc1, LoLuc1-3, HtLuc2, TurboLuc16 (TLuc), Renilla Luc, Firefly luciferase (FfLuc or Fluc), LucPPe-146-1H2, LucPPe-133-1B2, LucPPe-78-0B10, LucPPe49-7C6A, LucPpL-81-6G1, CBGRluc, thermostable alkaline phosphatase or homologs or orthologs or mutants or derivatives thereof for the purpose of use as a target cell line for measuring cytotoxicity. In various embodiments, the cell lines are modified and/or selected to lack the expression of one or more antigens.

Also provided herein are primary cells and/or compositions comprising primary cells wherein the primary cells express the non-secretory forms of GLuc, NLuc, MLuc7, HTLuc, PaLuc1, PaLuc2, MpLuc1, McLuc1, MaLuc1, MoLuc1, MoLuc2, MLuc39, PsLuc1, LoLuc1-3, HtLuc2, TurboLuc16 (TLuc), Renilla Luc, Firefly luciferase (FfLuc or Fluc), LucPPe-146-1H2, LucPPe-133-1B2, LucPPe-78-0B10, LucPPe49-7C6A, LucPpL-81-6G1 or CBGRluc or homologs or orthologs or mutants or derivatives thereof. In various embodiments, the primary cell lines are modified and/or selected to lack the expression of one or more antigens.

Further provided herein are target cells and/or compostions comprising target cells wherein the target cells express one or more of the antigens selected from a group consisting of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRviii); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); FmsLike Tyrosine Kinase 3 (FLT3); Tumor-associated glycoprotein 72 (TAG72); CD38; CD44v6; a glycosylated CD43 epitope expersed on acute leukemia or lymphoma but not on hematopoietic progenitors, a glycosylated CD43 epitope expressed on non-hematopoietic cancers, Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-llRa); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAlX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDClalp(1-4)bDGlcp(1-1)Cer); transglutaminase 5 (TGS5); high molecular weight-melanoma associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCT A-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B 1 (CYP1B 1); CCCTC-Binding Factor (Zinc Finger Protein)-Like (BORIS or Brother of the Regulator of imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1), MPL, Biotin, c-MYC epitope Tag, CD34, LAMP1 TROP2, GFRalpha4, CDH17, CDH6, NYBR1, CDH19, CD200R, Slea (CA19.9; Sialyl Lewis Antigen) Fucosyl-GM1, PTK7, gpNMB, CDH1-CD324, DLL3, CD276/B7H3, IL11Ra, IL13Ra2, CD179b-IGLll, ALK TCRgamma-delta, NKG2D, CD32 (FCGR2A), Tn ag, CSPG4-HMW-MAA, Tim1-/HVCR1, CSF2RA (GM-CSFR-alpha), TGFbetaR2, VEGFR2/KDR, Lews Ag, TCR-beta1 chain, TCR-beta2 chain, TCR-gamma chain, TCR-delta chain, FITC, Leutenizing hormone receptor (LHR), Follicle stimulating hormone receptor (FSHR), Chorionic Gonadotropin Hormone receptor (CGHR), CCR4, GD3, SLAMF6, SLAMF4, HIV1 envelope glycoprotein, HTLV1-Tax, CMV pp65, EBV-EBNA3c, influenza A hemagglutinin (HA), GAD, PDL1, Guanylyl cyclase C (GCC), KSHV-K8.1 protein, KSHV-gH protein, HLA, HLA-A, HLA-A2, HLA-B, HLA-C, HLA-DP, HLA-DM, HLA-DOA, HLA-DOB, HLA-DQ, HLA-DR or HLA-G.

In various embodiments, the cell lines, primary cells and/or target cells are modified and/or selected to lack the expression of one or more antigens selected from a group consisting of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRviii); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); FmsLike Tyrosine Kinase 3 (FLT3); Tumor-associated glycoprotein 72 (TAG72); CD38; CD44v6; a glycosylated CD43 epitope expersed on acute leukemia or lymphoma but not on hematopoietic progenitors, a glycosylated CD43 epitope expressed on non-hematopoietic cancers, Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-llRa); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAlX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDClalp(1-4)bDGlcp(1-1)Cer); transglutaminase 5 (TGS5); high molecular weight-melanoma associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCT A-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B 1 (CYP1B 1); CCCTC-Binding Factor (Zinc Finger Protein)-Like (BORIS or Brother of the Regulator of imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1), MPL, Biotin, c-MYC epitope Tag, CD34, LAMP1 TROP2, GFRalpha4, CDH17, CDH6, NYBR1, CDH19, CD200R, Slea (CA19.9; Sialyl Lewis Antigen) Fucosyl-GM1, PTK7, gpNMB, CDH1-CD324, DLL3, CD276/B7H3, IL11Ra, IL13Ra2, CD179b-IGLll, ALK TCRgamma-delta, NKG2D, CD32 (FCGR2A), Tn ag, CSPG4-HMW-MAA, Tim1-/HVCR1, CSF2RA (GM-CSFR-alpha), TGFbetaR2, VEGFR2/KDR, Lews Ag, TCR-beta1 chain, TCR-beta2 chain, TCR-gamma chain, TCR-delta chain, FITC, Leutenizing hormone receptor (LHR), Follicle stimulating hormone receptor (FSHR), Chorionic Gonadotropin Hormone receptor (CGHR), CCR4, GD3, SLAMF6, SLAMF4, HIV1 envelope glycoprotein, HTLV1-Tax, CMV pp65, EBV-EBNA3c, influenza A hemagglutinin (HA), GAD, PDL1, Guanylyl cyclase C (GCC), KSHV-K8.1 protein, KSHV-gH protein, HLA, HLA-A, HLA-A2, HLA-B, HLA-C, HLA-DP, HLA-DM, HLA-DOA, HLA-DOB, HLA-DQ, HLA-DR and HLA.

Further provided herein are kits comprising components for assessing cytotoxicity of an agent. In various embodiments, the kits include target cells (for example, cells engineered to express intracellularly one or more reporters and/or cell lines expressing one or more reporters and/or primary cells expressing one or more reporters), and substrates for activating the reporter. The target cells, reporters and substrates are as described herein. In various embodiments, the kits further comprise instructions for use of the kit.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1A-FIG. 1B depicts in accordance with various embodiments of the invention, that induction of cell death by treatment of HEL-92-1-17 and RAJI cells stably expressing GLuc (SEQ ID NO: 1) with Digitonin resulted in an increase in GLuc luciferase activity which was detectable even at 1 cell/well level and there was a progressive increase in luciferase activity with increase in cell number.

FIG. 2A-FIG. 2C depicts in accordance with various embodiments of the invention, that Digitonin treatment of NLuc expressing K562, HEL-91-1-17 and RAJI cells resulted in increase in NLuc activity which was detectable even at 1 cell/well level and there was a progressive increase in luciferase activity with increase in cell number.

FIG. 3A-FIG. 3B depicts in accordance with various embodiments of the invention, that induction of cell death by Digitonin results in increase in MLuc7 activity that was detectable even at 1 cell/well level and there was a progressive increase in MLuc7 activity with increase in cell number up to 400K cells/well.

FIG. 4 depicts in accordance with various embodiments of the invention, that induction of cell death by Digitonin and NK92MI cells resulted in increase in MLuc7 activity that was detectable even at 1 cell/well level and there was a progressive increase in MLuc7 activity with increase in cell number up to 10K cells/well.

FIG. 5A-FIG. 5B depicts in accordance with various embodiments of the invention, that Digitonin-treatment of K562 and RAJI cells stably expressing TurboLuc16 (TLuc) resulted in increase in TLuc activity.

FIG. 6 depicts in accordance with various embodiments of the invention, increase in Renilla Luc activity upon induction of death of K562 cells expressing Renilla-Luc by NK92MI cells or by treatment with Digitonin.

FIG. 7 depicts in accordance with various embodiments of the invention, that there is an increase in CBGR-Luc activity upon induction of death of CBGR-Luc-transfected 293FT cells following treatment with Digitonin.

FIG. 8 depicts in accordance with various embodiments of the invention, that there is a progressive increase in GLuc activity with increase in E:T ratio upon co-culture of K562-GLuc cells with NK92MI cells.

FIG. 9A-9C depicts in accordance with various embodiments of the invention, FIG. 9A: induction of K562-GLuc cell death by NK92MI cells results in significant and near equivalent increase in GLuc activity when measured either by directly adding 0.5×CTZ assay buffer to the wells containing cells plus supernatant or when added to supernatant that had been cleared of cells by a centrifugation step. FIG. 9B: supernatants from cell death assay containing GLuc and Renilla can be frozen and re-thawed after 2 weeks without loss of luciferase activities. FIG. 9C: GLuc assay conducted using the Renilla Luciferin substrate gave similar results as those obtained after addition of 0.5×CTZ assay buffer containing native coeloentrazine.

FIG. 10A-FIG. 10C depict in accordance with various embodiments of the invention, that there is a progressive and nearly equivalent increase in GLuc activity in both the supernatant fraction and the cells plus supernatant fraction with increase in number of K562-GLuc target cells upon co-culture with NK92MI effector cells (FIG. 10A and FIG. 10B). FIG. 10C depicts in accordance with various embodiment of the invention that there is slight increase in GLuc activity even in the cell pellet fraction with increase in number of K562-GLuc target cells upon co-culture with NK92MI effector cells.

FIG. 11A-FIG. 11B depicts in accordance with various embodiments of the invention, GLuc-based Matador cytotoxicity assay can be used to monitor cytotoxicity induced by CAR expressing effector cells.

FIG. 12 depicts in accordance with various embodiments of the invention, that induction of RAJI cell death by T cells expressing CD19-specific CAR results in increase in GLuc activity, whereas no increase in GLuc activity was observed by co-culture with uninfected T cells or those expressing a negative control CAR (i.e. 4C3) whose target antigen is not expressed in RAJI cells.

FIG. 13 depicts in accordance with various embodiments of the invention, inhibition of CD19MM-(vL-vH)-Myc-BBz-T2A-PAC(062915-D03)[SEQ ID NO:82]-CAR-T cells induced cell death by 50 nM and 100 nM Dasatinib (Das), while Imatinib (IM) has no effect.

FIG. 14 depicts in accordance with various embodiments of the invention, an increase in GLuc activity upon co-culture of PC3-GLuc cells with T cells infected with a lentivirus encoding a CAR targeting TROP2 (CD8SP-TROP2-h7E6-SVG-(vL-vH)-Myc-z-P2A-K13-Flag-T2A-PAC) as compared with co-culture with uninfected T cells (UI).

FIG. 15 depicts in accordance with various embodiments of the invention, that co-culture of primary leukemia blast cells that have been infected with a GLuc-encoding lentivirus with NK92MI cells expressing the FMC63-BBZ-CAR targeting the CD19 antigen or primary human T cells resulted in significant increase in GLuc activity.

FIG. 16 depicts in accordance with various embodiments of the invention, co-culture of RAJI cells transiently transfected with the non-secretory form of GLuc cDNA with T cells expressing an FMC63-BBz chimeric antigen receptor resulted in increase in GLuc activity as compared to uninfected T cells, indicative of cell death.

FIG. 17A-FIG. 17B depicts in accordance with various embodiments of the invention, FIG. 17A: co-culture of RAJI-NLuc cells with NK92MI cells expressing a FMC63-MYC-BBZ-T2A-EGFP chimeric antigen receptor resulted in significant increase in NLuc activity as compared to co-culture with NK92MI cells expressing a control (4C3-MYC-BBZ-T2A-EGFP) chimeric antigen receptor or culture without NK92MI cells (medium alone). FIG. 17B: co-culture of RAJI-NLuc cells with T cells expressing FMC63-MYC-BBZ-T2A-PAC chimeric antigen receptor resulted in significant increase in NLuc activity as compared to co-culture with T cells expressing a control (4C3-MYC-BBZ-T2A-PAC) chimeric antigen receptor or culture without T cells (medium alone)

FIG. 18 depicts in accordance with various embodiments of the invention, that treatment of BC3-pLenti-GLuc and BCBL1-pLenti-GLuc cells with drugs BIIB021 and JQ1 results in significant increase in GLuc activity present in the supernatant when normalized for the difference in the GLuc-expressing cells (as measured by GLuc activity in the cell lysates).

FIG. 19 depicts in accordance with various embodiments of the invention, that treatment of RAJI-GLuc cells with peripheral blood mononuclear cells in the presence of rituximab at both 1 μg/ml and 10 μg/ml concentrations resulted in increase in luciferase activity as compared to untreated cells, suggesting that the Matador assay can be used to monitor rituximab-induced cell death.

FIG. 20A-FIG. 20D depicts in accordance with various embodiments of the invention, FIG. 20A: co-culture of RAJI cells that had been stably transduced with MSCVhygro-GLuc vector with T cells in the presence of Blincyto (Blinatumomab) results in increase in luciferase activity as compared to untreated cells or cells treated with T cells or Binatumomab alone, demonstrating that the Matador assay can be used to monitor Binatumomab-induced cell death. FIG. 20B: increase in % specific lysis of RAJI-GLuc cells upon co-culture with T cells in the presence of Binatumomab. FIG. 20C: co-culture of RAJI-GLuc cells that had been stably transduced with pLenti-GLuc vector with T cells in the presence of Blincyto (Blinatumomab) results in increase in luciferase activity as compared to untreated cells or cells treated with T cells or Binatumomab alone, demonstrating that the GLuc-based Matador assay can be used to monitor Binatumomab-induced cell death. FIG. 20D: co-culture of RAJI cells stably expressing MLuc7 with T cells in the presence of Blincyto (Blinatumomab) results in increase in luciferase activity as compared to untreated cells or cells treated with T cells or Binatumomab alone, demonstrating that increase in MLuc7 activity can be used to monitor Binatumomab-induced cell death in Matador assay.

FIG. 21 depicts in accordance with various embodiments of the invention, GLuc-based Matador assay can be used to measure cytotoxicity of targeted agents (e.g. Imatinib (or Gleevec) and Dasatinib) as well as conventional chemotherapy drugs (e.g. doxorubicin, vincristine and vinblastine).

FIG. 22 depicts in accordance with various embodiments of the invention, treatment with Digitonin resulted in increase in luciferase activity in Digitonin-treated samples of 293FT cells that had been transiently transfected with non-secretory forms of luciferases from different copepods. The results demonstrate that similar to GLuc non-secretory forms of luciferases from other copepods can be used in Matador assay.

FIG. 23 depicts in accordance with various embodiments of the invention, induction of cell death by treatment with Digitonin resulted in increase in luciferase activity in case of 293FT cells transiently transfected with GLuc, TurboLuc16, NLuc, Renilla (RLuc), PaLuc1, HTluc1, LoLuc, Lucia Luc and MLuc7.

FIG. 24 depicts in accordance with various embodiments of the invention, that treatment with Digitonin resulted in increase in Luciferase activity in case of 293FT cells transiently transfected with the MYR-GGS-GLuc construct.

FIG. 25 depicts in accordance with various embodiments of the invention, increase in GLuc activity upon co-culture of KSHV-infected MM cells stably expressing GLuc with NK92MI cells suggesting that Matador assay can be used to monitor death of virally infected cells.

FIG. 26 depicts in accordance with various embodiments of the invention, Brentuximab vedotin treatment of GLuc-expressing Jurkat, KMH2, L428 cells led to a significant increase in GLuc activity in cell supernatant when normalized for the GLuc activity in cell lysates, demonstrating the utility of Matador assay for monitoring cytotoxicity induced by an antibody drug conjugate.

FIG. 27 depicts in accordance with various embodiments of the invention, that co-culture of peripheral blood mononuclear cells infected with a chimeric antigen receptor (CAR) targeting MPL and co-expressing GLuc with HEL cells expressing the MPL protein results in increase in GLuc activity, thereby demonstrating the utility of the Matador assay for monitoring activation-induced cell death.

FIG. 28A-C depicts in accordance with various embodiments of the invention, FIG. 28A: Digitonin-induced cell death of GFP transfected 293FT cells resulted in increase in GFP release in the supernatant fraction as measured by Western blotting with an antibody against GFP. FIG. 28B: Digitonin-induced cell death of mCherry-HA transfected 293FT cells resulted in increase in mCherry release in the supernatant fraction as measured by Western blotting with an antibody against the HA tag. FIG. 28C: significant increase in fluorescence in cell supernatant collected from cells that had been transfected with vectors encoding GFP and mCherry-HA, respectively, and then treated with Digitonin as compared to untreated cells.

FIG. 29 depicts in accordance with various embodiments of the invention, increase in % specific lysis of RAJI-NLuc cells when co-cultured with CD19-directed FMC63-Myc-BBZ and FMC63-Myc-CD28z CAR constructs.

FIG. 30A-C, depict in accordance with various embodiments of the invention, the effect of different assay conditions and sample types on production of glow-type luminescence by NLuc when used in Matador cytotoxicity assay.

FIG. 31 depicts in accordance with various embodiments of the invention, the effect of addition of bovine serum albumin on production of glow-type luminescence by NLuc when used in Matador cytotoxicity assay.

FIG. 32 depicts in accordance with various embodiments of the invention, that there was a significant increase in GLuc activity between the pre- and post-treatment serum samples of mice injected with RAJI-GLuc cells and then treated with NK92MI cells expressing chimeric antigen receptors targeting CD19. These results demonstrate that the Matador assay can be used to monitor cytotoxicity in vivo.

FIG. 33 depicts in accordance with various embodiments of the invention, treatment with Digitonin resulted in increase in luciferase activity in case of 293FT cells transiently transfected with the FfLuc construct.

FIG. 34 depicts in accordance with various embodiments of the invention, treatment with Digitonin resulted in increase in both FfLuc and GLuc activities in cell lines coexpressing both FfLuc and GLuc.

FIG. 35 depicts in accordance with various embodiments of the invention, stability of the exemplary luciferases at 37° C. that can be used in the Matador cytotoxicity assay. The results confirm that FfLuc has short half-life at 37° C. when used in the Matador cytotoxicity assay.

FIG. 36A-B depicts in accordance with various embodiments of the invention, FIG. 36A: increase in luciferase activity upon induction of cell death by co-culture of K562 cells stably expressing LucPPe-146-1H2 with NK92MI cells or by treatment with Digitonin. FIG. 36B: treatment with Digitonin results in increase in luciferase activity in case of 293FT cells transiently transfected with the LucPPe-146-1H2 expression construct.

FIG. 37 depicts in accordance with various embodiments of the invention, stability of the LucPPe-146-1H2 luciferase in the cell culture supernatant at 37° C. under the conditions of Matador cytotoxicity assay.

FIG. 38A-D depicts in accordance with various embodiments of the invention, that LucPPe-146-1H2 and NLuc can be combined to develop a dual luciferase based Matador cytotoxicity assay.

FIG. 39 depicts in accordance with various embodiments of the invention, co-culture of CD19^{-/-} clones of RAJI cells expressing NLuc with Blinatumomab in the presence of T cells fails to result in increase in NLuc activity thereby demonstrating that the Matador cytotoxicity assay can be used to rule out off-target cytotoxicity of an agent.

FIG. 40 depicts in accordance with various embodiments of the invention, treatment with Digitonin resulted in increase in embryonic alkaline phosphatase (EAP) activity in case of 293FT cells transiently transfected with an EAP expression construct.

### DETAILED DESCRIPTION OF INVENTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, NY 2006); Smith, March's Advanced Organic Chemistry Reactions, Mechanisms and Structure 7^{th} ed., J. Wiley & Sons (New York, NY 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, NY 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor NY, 2013); Kohler and Milstein, Derivation of specific antibody-producing tissue culture and tumor lines by cell fusion, Eur. J. Immunol. 1976 July, 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein, in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

### Definitions

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

As used herein, the term “about” refers to a measurable value such as an amount, a time duration, and the like, and encompasses variations of ±20%, ±10%, ±5%, ±1%, ±0.5% or ±0.1% from the specified value.

As used herein, “matador assay” or “matadaor cytotoxicity assay” refers to an assay of cellular cytotoxicity which involves the measurement of activity of a reporter that is not expressed endogenously by the target cell or is expressed endogenously by the target cell at a level much lower than what is achieved by engineered expression. The assay takes advantage of the fact that non-viable cells lose cell membrane integrity and either leak the reporter into the surrounding media or allow enhanced entry of the substrate of the reporter into the cell or both, resulting in an enhanced interaction between the reporter and its substrate, which in turn, results in an increase in the reporter activity. The preferred reporter for use in the matador assay is non-secretory form of a thermostable luciferase, such as GLuc, NLuc, PaLuc1, TLuc, MLuc7, Lucia Luc and LucPPe-146-1H2. The activity of the reporter in the Matador assay can be assayed in a homogenous format (i.e. single step) by adding the assay reagent containing the reporter substrate to the cell culture media containing the target cells. In an alternate embodiment, the activity of the reporter in the Matador assay can be assayed by adding the assay reagent containing the reporter substrate to the cell culture media that has been separated from the target cells (i.e. supernatant).

As used herein “non-vector method” refers to a method of introducing and/or expressing a reporter in a cell that does not involve the use of a vector. Several non-vector methods of gene transfer and expression are known in the art, such as transfection of naked DNA or electroporation of in vitro transcribed RNA.

As used herein, “foreign promoter” refers to a promoter that is not naturally present in the target cell expressing the reporter of the invention. Examples of foreign promoters include CMV immediate early promoter and SV40 promoter. The expression of TurboLuc-16 reporter is driven by CMV promoter in the exemplary vector pLENTI-TurboLuc-16-X3Flag-Blast-C04 (SEQ ID NO: 93).

As used herein, “natural promoter” refers to promoter that is naturally present in the target cell expressing the reporter of the invention. Examples of natural promoters include Elongation Factor 1α (EF1α) promoter and beta actin promoter. The natural promoter may be introduced into the target cells as part of the vector encoding the reporter of the invention. For example, the expression of LucPPe-146-1H2 reporter is driven by EF1α promoter in the vector pLenti-EF1-LucPPe-146-1H2-Flag-Pac-R01 (SEQ ID NO: 100). Alternatively, the DNA fragment encoding the reporter of the invention could be inserted downstream of an endogenously occurring natural promoter by homologous recombination. For example, GLuc cDNA could be inserted downstream of one of the genomic copies of the endogenous EF1α promoter using techniques of homologous recombination known in the art.

As used herein, “cell line” refers to cells that demonstrate the potential for indefinite subculture in vitro. A cell line is generally derived from one cell or set of cells of the same type. Examples of cell lines include K562, RAJI, Jurkat and HeLa.

As used herein, “primary cells” refers to cells derived directly from the parent tissue. Primary cells generally have the same karyotype and chromosome number as those in the original tissue. Examples of primary cells include primary T cells, primary B cells and primary vascular endothelial cells.

“Chimeric antigen receptor” or “CAR” or “CARs” as used herein refers to engineered receptors, which graft an antigen specificity onto cells (for example T cells such as naïve T cells, central memory T cells, effector memory T cells or combination thereof). CARs are also known as artificial T-cell receptors, chimeric T-cell receptors, or chimeric immunoreceptors. As used herein, the term “CAR” or “CARs” also encompasses newer approaches to conferring antigen specificity onto cells, such as Antibody-TCR chimeric molecules or AbTCR (WO 2017/070608 A1), TCR receptor fusion proteins or TFP (WO 2016/187349 A1) and Synthetic Immune Receptors or SIR (U.S. 62/429,597).

“Linker” (L) or “linker domain” or “linker region” as used herein refer to an oligo- or polypeptide region from about 1 to 100 amino acids in length, which links together two or more domains of the invention. Linkers may be composed of flexible residues like glycine and serine so that the adjacent protein domains are free to move relative to one another. Longer linkers may be used when it is desirable to ensure that two adjacent domains do not sterically interfere with one another. Linkers may be cleavable or non-cleavable. Examples of cleavable linkers include 2A linkers, for example T2A (SEQ ID NO: 66), P2A (SEQ ID NO: 67) and E2A (SEQ ID NO: 69), 2A-like linkers or functional equivalents thereof and combinations thereof. Other linkers will be apparent to those of skill in the art and may be used in connection with alternate embodiments of the invention.

“Co-express” as used herein refers to simultaneous expression of two or more genes. Genes may be nucleic acids encoding, for example, a single protein or a chimeric protein as a single polypeptide chain.

“Single chain variable fragment”, “single-chain antibody variable fragments” or “scFv” antibodies as used herein refer to forms of antibodies comprising the variable regions of only the heavy (V_{H}) and light (V_{L}) chains, connected by a linker peptide. The scFvs are capable of being expressed as a single chain polypeptide. The scFvs retain the specificity of the intact antibody from which it is derived. The light and heavy chains may be in any order, for example, V_{H}-linker-V_{L} or V_{L}-linker-V_{H}, so long as the specificity of the scFv to the target antigen is retained.

“Cancer” and “cancerous” refer to or describe the physiological condition in mammals that is typically characterized by unregulated cell growth. The term “cancer” is meant to include all types of cancerous growths or oncogenic processes, metastatic tissues or malignantly transformed cells, tissues, or organs, irrespective of histopathologic type or stage of invasiveness.

It is to be inferred without explicit recitation and unless otherwise intended, that when the present disclosure relates to a polypeptide, protein, polynucleotide, antibody or fragment thereof, an equivalent or a biologically equivalent of such is intended within the scope of this disclosure. As used herein, the term “biological equivalent thereof” is intended to be synonymous with “equivalent thereof” when referring to a reference protein, antibody or fragment thereof, polypeptide or nucleic acid, intends those having minimal homology while still maintaining desired structure or functionality. Unless specifically recited herein, it is contemplated that any of the above also includes equivalents thereof. For example, an equivalent intends at least about 70% homology or identity, or at least 80% homology or identity and alternatively, or at least about 85%, or alternatively at least about 90%, or alternatively at least about 95%, or alternatively at least 98% percent homology or identity and exhibits substantially equivalent biological activity to the reference protein, polypeptide, antibody or fragment thereof or nucleic acid. Alternatively, when referring to polynucleotides, an equivalent thereof is a polynucleotide that hybridizes under stringent conditions to the reference polynucleotide or its complement. Alternatively, when referring to polypeptides or proteins, an equivalent thereof is a expressed polypeptide or protein from a polynucleotide that hybridizes under stringent conditions to the polynucleotide or its complement that encodes the reference polypeptide or protein.

Cloning and expression methods will be apparent to a person of skill in the art and may be as described in WO 2015/142675; Sambrook et al., 2012, MOLECULAR CLONING: A LABORATORY MANUAL, volumes 1-4, Cold Spring Harbor Press, NY; June et al. 2009 Nature Reviews Immunology 9.10: 704-716; WO 01/96584; WO 01/29058; U.S. Pat. No. 6,326,193, the contents of each of which are herein incorporated by reference in their entirety as though set forth herein.

Physical methods for introducing polynucleotides of into host cells such as calcium phosphate transfection and the like are well known in the art and will be apparent to a person of skill in the art. In exemplary embodiments, such methods are set forth in Sambrook et al., 2012, MOLECULAR CLONING: A LABORATORY MANUAL, volumes 1-4, Cold Spring Harbor Press, NY); U.S. Pat. Nos. 5,350,674 and 5,585,362, the contents of each of which are herein incorporated by reference in their entirety as though set forth herein.

As used herein, “amino acid” is meant to include both natural and synthetic amino acids, and both D and L amino acids. “Standard amino acid” means any of the twenty L-amino acids commonly found in naturally occurring peptides. “Nonstandard amino acid” means any amino acid, other than the standard amino acids, regardless of whether it is prepared synthetically or derived from a natural source.

Herein, “peptide” and “protein” are used interchangeably, and refer to a compound comprised of at least two amino acid residues covalently linked by peptide bonds or modified peptide bonds (e.g., peptide isosteres). No limitation is placed on the maximum number of amino acids which may comprise a protein or peptide. The amino acids comprising the peptides or proteins described herein and in the appended claims are understood to be either D or L amino acids with L amino acids being preferred. The amino acid comprising the peptides or proteins described herein may also be modified either by natural processes, such as posttranslational processing, or by chemical modification techniques which are well known in the art. Modifications can occur anywhere in a peptide, including the peptide backbone, the amino acid side-chains and the amino or carboxyl termini. It is understood that the same type of modification may be present in the same or varying degrees at several sites in a given peptide. Also, a given peptide may contain many types of modifications. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. See, for instance, Proteins--Structure and Molecular Properties, 2nd Ed., T. E. Creighton, W.H. Freeman and Company, New York, 1993 and Wold F, Posttranslational Protein Modifications: Perspectives and Prospects, pgs. 1-12 in Posttranslational Covalent Modification of Proteins, B. C. Johnson, Ed., Academic Press, New York, 1983; Seifter et al., “Analysis for protein modifications and nonprotein cofactors,” Meth. Enzymol. (1990) 182: 626-646 and Rattan et al. (1992), “Protein Synthesis: Posttranslational Modifications and Aging,” Ann NY Acad Sci 663: 48-62.

“Polynucleotide” as used herein includes but is not limited to DNA, RNA, cDNA (complementary DNA), mRNA (messenger RNA), rRNA (ribosomal RNA), shRNA (small hairpin RNA), snRNA (small nuclear RNA), snoRNA (short nucleolar RNA), miRNA (microRNA), genomic DNA, synthetic DNA, synthetic RNA, and/or tRNA.

As used herein, the term “polypeptide” encompasses amino acid chains of any length, including full length proteins (i.e., antigens), wherein the amino acid residues are linked by covalent peptide bonds.

A polypeptide “variant,” as used herein, is a polypeptide that differs from the recited polypeptide only in conservative substitutions and/or modifications, such that the therapeutic, antigenic and/or immunogenic properties of the polypeptide are retained. Polypeptide variants preferably exhibit at least about 70%, more preferably at least about 90% and most preferably at least about 95% homology to the identified polypeptides. For polypeptides with immunoreactive properties, variants can, alternatively, be identified by modifying the amino acid sequence of one of the above polypeptides, and evaluating the immunoreactivity of the modified polypeptide. Such modified sequences can be prepared and tested using, for example, the representative procedures described herein.

As used herein, a “conservative substitution” is one in which an amino acid is substituted for another amino acid that has similar properties, such that one skilled in the art of peptide chemistry would expect the secondary structure and hydropathic nature of the polypeptide to be substantially unchanged. In general, the following groups of amino acids represent conservative changes: (1) ala, pro, gly, fly asp, gln, asn, set, thr; (2) cys, ser, fyr, thr; (3) val, ile, leu, met, ala, phe; (4) lys, arg, his; and (5) phe, tyr, trp, his.

Variants can also, or alternatively, contain other modifications, including the deletion or addition of amino acids that have minimal influence on the antigenic properties, secondary structure and hydropathic nature of the polypeptide. For example, a polypeptide can be conjugated to a signal (or leader) sequence at the N-terminal end of the protein which co-translationally or post-translationally directs transfer of the protein. The polypeptide can also be conjugated to a linker or other sequence for ease of synthesis, purification or identification of the polypeptide (e.g., poly-His), or to enhance binding of the polypeptide to a solid support. For example, a polypeptide can be conjugated to an immunoglobulin Fc region.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

### RNA Transfection

The nucleic acids encoding the reporters of the invention can be introduced into target cells using RNA transfection. Methods for producing an in vitro transcribed RNA reporters are disclosed herein. A method for generating mRNA for use in transfection can involve in vitro transcription (IVT) of a template with specially designed primers, followed by poly A addition, to produce a construct containing 3′ and 5′ untranslated sequence (“UTR”), a 5′ cap and/or Internal Ribosome Entry Site (IRES), the nucleic acid to be expressed, and a poly A tail, typically 50-2000 bases in length. RNA so generated can efficiently transfect different kinds of cells. In one aspect, the template includes sequences for the reporter.

In one aspect, a reporter (e.g., GLuc or NLuc) of the present invention is encoded by an mRNA or an in vitro transcribed RNA (IVTR). In one aspect, the IVTR encoding a reporter described herein is introduced into a target cell, e.g., a primary cell or a cell line, for production of a reporter-expressing cell for use in the Matador assays as described herein.

In one embodiment, the in vitro transcribed RNA reporter can be introduced to a cell using transient transfection. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, plasmid DNA, phage DNA, cDNA, synthetic DNA sequence or any other appropriate source of DNA. The desired template for in vitro transcription is a reporter, such as GLuc or NLuc coding sequence lacking the signal peptide, described herein.

PCR is used to generate a template for in vitro transcription of mRNA which is used for transfection. Methods for performing PCR are well known in the art. Any DNA polymerase useful for PCR can be used in the methods disclosed herein. The reagents and polymerases are commercially available from a number of sources.

Chemical structures with the ability to promote stability and/or translation efficiency may also be used. The RNA preferably has 5′ and 3′ UTRs. In one embodiment, the 5′ UTR is between one and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the nucleic acid of interest. Alternatively, UTR sequences that are not endogenous to the nucleic acid of interest can be added by incorporating the UTR sequences into the forward and reverse primers or by any other modifications of the template. The use of UTR sequences that are not endogenous to the nucleic acid of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous nucleic acid. Alternatively, when a 5′ UTR that is not endogenous to the nucleic acid of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be 5′UTR of an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one preferred embodiment, the promoter is a T7 polymerase promoter. Other useful promoters include, but are not limited to, T3 and SP6 RNA polymerase promoters. Consensus nucleotide sequences for T7 and SP6 promoters are known in the art.

In an embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100 T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines. Poly(A) tails of RNAs can be further extended following in vitro transcription with the use of a poly(A) polymerase, such as E. coli poly A polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps on also provide stability to RNA molecules. In a preferred embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

An internal ribosome entry site (IRES) sequence can be present in the RNAs produced by the methods disclosed herein. The IRES sequence may be any viral, chromosomal or artificially designed sequence which initiates cap-independent ribosome binding to mRNA and promotes the initiation of translation. Any solutes suitable for cell electroporation, which can contain factors facilitating cellular permeability and viability such as sugars, peptides, lipids, proteins, antioxidants, and surfactants can be included.

RNA can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001) or by causing transient perturbations in cell membranes using a microfluidic device (see, for example, patent applications WO 2013/059343 A1 and PCT/US2012/060646).

### Vectors

Vectors which may be used to express the reporters of the invention include but are not limited to lentivirus vectors, gamma retrovirus vectors, foamy virus vectors, AAV vectors, adeno virus vectors, engineered hybrid viruses, (including but not limited to transposon mediated vectors, such as Sleeping Beauty, Piggybak, and Integrases such as Phi31.

The term “recombinant DNA molecule”, as used herein, refers to a DNA molecule which is comprised of segments of DNA joined together by means of molecular biological techniques known to one of skill in the art, including but not limited to genetic recombination (i.e., molecular cloning).

The term “recombinant protein” or “recombinant polypeptide”, as used herein, refers to a protein molecule which is expressed from a recombinant DNA molecule.

“Recombinant virus” refers to a virus that has been genetically altered (e.g., by the addition or insertion of a heterologous nucleic acid construct into the particle).

A reporter transgene may also include regulatory sequences and intron regions. Promoters that would regulate reporter expression may include constitutive, inducible and tissue-specific promoters. The term “promoter region” is used herein in its ordinary sense to refer to a nucleotide region including a DNA regulatory sequence, wherein the regulatory sequence is derived from a gene which is capable of binding RNA polymerase and initiating transcription of a downstream (3′-direction) coding sequence. The promoter region may comprise of or be linked to additional regulatory sequences, such as enhancers, that modulate the expression of the reporter transgene. The preferred promoter for expressing the reporters of the invention is a strong constitutive promoter, e.g., Elongation Factor α promoter or a CMV immediate early promoter.

A “gene” or “coding sequence” or a sequence which “encodes” a particular protein is a nucleic acid molecule that is transcribed (in the case of DNA) and translated (in the case of mRNA) into a polypeptide in vitro or in vivo when placed under the control of appropriate regulatory sequences.

The term “transfection” is used herein to refer to the uptake of foreign DNA or RNA by a cell. A cell has been “transfected” when exogenous DNA or RNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous DNA moieties, such as a plasmid vector and other nucleic acid molecules, into suitable host cells. The term refers to both stable and transient uptake of the genetic material.

The term “transduction” denotes the delivery of a DNA or RNA molecule to a recipient cell either in vivo or in vitro, via any method of gene delivery, including viral vector delivery.

The term “transformation” as used herein refers to a change in a cell's genetic characteristics, and a cell has been transformed when it has been modified to contain a new DNA. For example, a cell is transformed where it is genetically modified from its native state. Following transfection, the transforming nucleic acid can recombine with that of the cell by physically integrating into a chromosome of the cell, can be maintained transiently as an episomal element without being replicated, or can replicate independently as a plasmid. A cell is considered to have been stably transformed when the transforming nucleic acid is replicated with the division of the cell.

Various methods produce stable transfectants which express the reporters of the invention. In one embodiment, a method of stably transfecting and re-directing cells is by electroporation using naked DNA. By using naked DNA, the time required to produce redirected cells may be significantly reduced. Additional methods to genetically engineer cells include but are not limited to chemical transformation methods (e.g., using calcium phosphate, dendrimers, liposomes and/or cationic polymers), non-chemical transformation methods (e.g., electroporation, optical transformation, gene electrotransfer, transient perturbation in cell membranes and/or hydrodynamic delivery) and/or particle-based methods (e.g., impalefection, using a gene gun and/or magnetofection). Viral transduction methods may also be used to generate redirected cells which express the reporters of the invention.

As used herein an “expression vector” refers to a DNA molecule, or a clone of such a molecule, which has been modified through human intervention to contain segments of DNA combined and juxtaposed in a manner that would not otherwise exist in nature. DNA constructs can be engineered to include a first DNA segment encoding an acetylation-resistant engineered PDCL3 polypeptide described herein operably linked to additional DNA segments encoding a desired recombinant protein of interest. In addition, an expression vector can comprise additional DNA segments, such as promoters, transcription terminators, enhancers, and other elements. One or more selectable markers can also be included. DNA constructs useful for expressing cloned DNA segments in a variety of prokaryotic and eukaryotic host cells can be prepared from readily available components or purchased from commercial suppliers.

After transfection, the host cell can be maintained either transiently transformed or stably transformed with said nucleic acid or expression vector. Introduction of multiple nucleic acids or expression vectors, and selection of cells containing the multiple nucleic acids or expression vectors can be done either simultaneously or, more preferably, sequentially. The technique of establishing a cell line stably transformed with a genetic material or expression vector is well known in the art (Current Protocols in Molecular Biology). In general, after transfection, the growth medium will select for cells containing the nucleic acid construct by, for example, drug selection or deficiency in an essential nutrient, which is complemented by a selectable marker on the nucleic acid construct or co-transfected with the nucleic acid construct. Cultured mammalian cells are generally cultured in commercially available serum-containing or serum-free medium. Selection of a medium appropriate for the particular host cell used is within the level of ordinary skill in the art.

By “cell culture” or “culture” is meant the growth and propagation of cells outside of a multicellular organism or tissue. Suitable culture conditions for mammalian cells are known in the art. See e.g. Animal cell culture: A Practical Approach, D. Rickwood, ed., Oxford University Press, New York (1992). Mammalian cells can be cultured in suspension or while attached to a solid substrate. Fluidized bed bioreactors, hollow fiber bioreactors, roller bottles, shake flasks, or stirred tank bioreactors, with or without microcarriers, can be used.

As used herein, “cell culture medium” is a media suitable for growth of animal cells, such as mammalian cells, in in vitro cell culture. Cell culture media formulations are well known in the art. Typically, cell culture media are comprised of buffers, salts, carbohydrates, amino acids, vitamins and trace essential elements. “Serum-free” applies to a cell culture medium that does not contain animal sera, such as fetal bovine serum. Various tissue culture media, including defined culture media, are commercially available, for example, any one or a combination of the following cell culture media can be used: RPMI-1640 Medium, RPMI-1641 Medium, Dulbecco's Modified Eagle's Medium (DMEM), Minimum Essential Medium Eagle, F-12K Medium, Ham's F12 Medium, Iscove's Modified Dulbecco's Medium, McCoy's 5A Medium, Leibovitz's L-15 Medium, and serum-free media such as EX-CELL™ 300 Series (JRH Biosciences, Lenexa, Kans.), among others. Serum-free versions of such culture media are also available.

Suitable selectable markers for drug selection used with the compositions and methods described herein include, but are not limited to, neomycin (G418), hygromycin, puromycin, zeocin, colchine, methotrexate, and methionine sulfoximine.

Once a drug resistant cell population is established, individual clones may be selected and screened for high expressing clones. Methods of establishing cloned cell line are well known in the art, including, but not limited to, using a cloning cylinder, or by limiting dilution. Expression of the recombinant protein of interest from each clone can be measured by methods such as, but not limited to, immunoassay, enzymatic assay, or chromogenic assay. A cell line stably transformed with a first nucleic acid construct may be then used as host cell for transfection with a second or more nucleic acid constructs, and subjected to different drug selections.

The terms “T-cell” and “T-lymphocyte” are interchangeable and used synonymously herein. Examples include but are not limited to naïve T cells, central memory T cells, effector memory T cells or combinations thereof.

The term “thermostable” generally refers to the resilience of a substance to relatively high temperature treatment. A thermostable enzyme is an enzyme that retains its definitive enzymatic activity despite exposure to relatively high temperature. A thermostable reporter is a reporter that retains its definitive reporter activity despite exposure to relatively high temperature. As most cytotoxic assays are carried out at temperature of 37° C., a thermostable reporter suitable for cytotoxic assay of the invention has a half-life (the length of time it takes for a reporter to lose one half of its initial activity) of greater than 10 min; of greater than 30 min; of greater than 60 min; of greater than 2 hours; of greater than 4 hours; of preferably greater than 8 hour; of more preferably greater than 24 hours; of even more preferably greater than 96 hours at a temperature of 37° C. under tissue culture conditions used in the cytotoxicity assay.

The term “functional” when used in conjunction with “derivative” or “variant” or “fragment” refers to a polypeptide which possess a biological activity that is substantially similar to a biological activity of the entity or molecule of which it is a derivative or variant or fragment thereof.

“Luciferase” or “Luciferases” as used herein refer class of oxidative enzymes that produce bioluminescence. The non-secretory form of luciferases for use in the compositions and methods described herein lack the N-terminal secretory sequence but produce bioluminescence. In the preferred embodiment, the non-secretory form of luciferase is stable under the cell culture conditions of the Matador assay. In the preferred embodiment, the non-secretory form of luciferase is thermostable under the cell culture conditions of the Matador assay. Methods of generating thermostable luciferases are known in the art (e.g, PCT/US99/30925). In exemplary embodiments, the luciferase is obtained from copepods, deep sea shrimp, beetle or homologs or orthologs thereof or mutants or derivatives thereof. In some embodiments, the copepods are any one or more of Gaussia princeps, Pleuromamma abdominalis, Metridia pacifica, Metridia curticauda, Metridia asymmetrica, Metridia okhotensis, Metridia longa, Lucicutia ovaliformis, Heterorhabdus tanneri, and Pleuromamma scutullata. In some embodiments, the beetles are any one or more of Photuris Pennsylvanica and Pyrophorus plagiothalamus. In exemplary embodiments, the luciferases are any one or more of GLuc, NanoLuc (NLuc), MLuc7, HtLuc, LoLuc, PaLuc1, PaLuc2, MpLuc1, McLuc1, MaLuc1, MoLuc1, MoLuc2, MLuc39, PsLuc1, LocLuc1-3, HtLuc2 Renilla, TurboLuc16 (TLuc), Lucia-Luc, Flue or FfLuc (Firefly Luc), LucPpe1, LucPpe2, LucPPe-146-1H2, LucPPe-133-1B2, LucPPe-78-0B10, LucPPe49-7C6A, LucPpL-81-6G1, CBGRluc or any of the thermostable luciferases described in PCT/US99/30925 or homologs or orthologs thereof or mutants or functional derivatives or variants thereof. In various embodiments, mutants or functional derivatives of the luciferases retain at least 100%, 90%, 80%, 70%, 60%, 50%, 40%, 30%, 20%, 10%, 5% of the bioluminescence activity of the parent luciferase from which the mutant or function derivative is derived. As described in Takenaka et al (Evolution of bioluminescence in marine planktonic copepods. 2012 Mol Biol Evol 29(6): 1669-1681), the copepod luciferases comprise two domains and each domain includes conserved sequences across the various luciferases. In some embodiments, any luciferase comprising the consensus sequence C-x(3)-C-L-x(2)-L-x(4)-C-x(8)-P-x-R-C(SEQ ID NO: 101) in each of domains 1 and 2 as described by Takenaka et al., may be used in the compositions and methods described herein. In some embodiments, any luciferase comprising the consensus sequence K-x(7)-E-M-E-A-N-A-x(3)-G-C-x-R-x-C-L-I-x-L-S-x-I-K-C-T-x-K-M-x(4)-P-G-R-C-H-X-Y-x(8)-G (SEQ ID NO: 102) in domain 1 as described by Takenaka et al. and consensus sequences I-x-G-x(6)-M-x-Q-F-x(2)-Q-V-x(2)-C-x(2)-C-x(3)-C-L-K-G-L-A-N-x(2)-C-S-x(2)-L-x(3)-L-P-x-R-C-x(2)-F-x(3)-I-X(8)-G (SEQ ID NO: 103) in domain 2 as described by Takenaka et al., may be used in the compositions and methods described herein. In various embodiments, the functional fragments or mutants of luciferases for use with the compositions or methods described herein comprise the consensus sequence C-x(3)-C-L-x(2)-L-x(4)-C-x(8)-P-x-R-C (SEQ ID NO: 101) in domain 1 as described by Takenaka et al. In various embodiments, the functional fragments or mutants of luciferases for use with the compositions or methods described herein comprise the consensus sequence C-x(3)-C-L-x(2)-L-x(4)-C-x(8)-P-x-R-C (SEQ ID NO: 101) in domain 2 as described by Takenaka et al. In various embodiments, the functional fragments or mutants of luciferases for use with the compositions or methods described herein comprise the consensus sequence C-x(3)-C-L-x(2)-L-x(4)-C-x(8)-P-x-R-C (SEQ ID NO: 101) in domain 1 and in domain 2, as described by Takenaka et al.

The term “reporter” means a gene or a protein whose product is easily and quantifiably assayed when expressed in a cell. The introduction and expression of reporters into cells is described in U.S. Pat. No. 5,298,429, incorporated herein by reference. Representative reporters include β-galactosidase (lacZ), chloramphenicol acetyltransferase (cat), β-glucuronidase (GUS), and the like, and luciferase. A preferred reporter of the invention is a thermostable luciferase.

As the term is used herein, a “cytotoxicity assay” refers to an assay for the quantitative or qualitative detection of the death of a cell. Examples of cytotoxicity assays include LDH release assay and Cr⁵¹ release assay. A cytotoxicity assay generally involves exposure of target cell(s) to an agent or agents capable to modulating cell death for a period of time ranging for a few seconds to several days followed by quantitative or qualitative detection of the death of the exposed cell(s).

As the term is used herein, a “bioluminescence assay” or “luciferase assay” refers to an assay for the quantitative or qualitative detection of the luciferase protein by its enzymatic activity.

As used herein, bioluminescence substrate refers to the compound that is oxidized in the presence of a luciferase, and any necessary activators, and generates light. These substrates are referred to as luciferins herein, are substrates that undergo oxidation in a bioluminescence reaction. These bioluminescence substrates include any luciferin or analog thereof or any synthetic compound with which a luciferase interacts to generate light. Preferred substrates are those that are oxidized in the presence of a luciferase or protein in a light-generating reaction. Bioluminescence substrates, thus, include those compounds that those of skill in the art recognize as luciferins. Luciferins, for example, include firefly luciferin, coelenterazine, bacterial luciferin, as well as synthetic analogs of these substrates or other compounds that are oxidized in the presence of a luciferase in a reaction that produces bioluminescence. As described in Hall M P et al, ACS Chemical Biology, bioluminescence is found across a diversity of life that includes bacteria, insects, fungi, and an abundance of marine organisms. It occurs when a photon-emitting substrate (luciferin) is oxidized by a generic class of enzymes called luciferases. These enzymes have been popular as reporters of cellular physiology because of their ability to provide highly sensitive quantitation with broad linearity. Firefly (FLuc, or FfLuc, 61 kDa) and Renilla (RLuc, 36 kDa) luciferases have accounted for the majority of such applications, particularly for elucidating molecular processes coupled to gene expression. More recently, bioluminescence has been applied to other aspects of cellular analysis. The widely recognized utility of bioluminescence has spurred investigation of alternative luciferases, predominantly from marine organisms. Luciferase genes have been derived from the copepods Gaussia princeps (20 kDa) and Metridia longa (24 kDa), the ostracod Cypridina noctiluca (61 kDa), the dinoflagellate Pyrocystis lunula (40 kDa), and the deep sea shrimp, Oplophorus gracilirostris (106 kDa). Takenaka Y et al. has provided a description of the structure and evolutionary history of copepod luciferase genes (Takenaka, Yamaguchi et al. 2012). They classified Copepod luciferases into two groups of Metridinidae and Heterorhabdidae/Lucicutiidae families based on phylogenetic analyses, with confirmation of the interrelationships within the Calanoida using 18S ribosomal DNA sequences. The specific activity of luciferases isolated from copepods in the family Metridinidae (MpLuc1, MpLuc2, MoLuc1, MoLuc2, PaLuc1, and PaLuc2) was notably higher than that of luciferases from the Heterorhabdidae family (HtLuc1, HtLuc2, HmLuc1, and HmLuc2). LoLuc, isolated from L. ovaliformis, which belongs to the Lucicutiidae family, showed intermediate levels of activity. Two catalytic domains had been identified in Gaussia luciferase (Inouye and Sahara 2008), and Takenake Y et al confirmed the presence of two short repeat sequences in the primary structures of the novel copepod luciferases isolate by them (Takenaka, Yamaguchi et al. 2012). Alignment of two repeat sequences consisting of 62-64 amino acid residues (referred as domains 1 and 2, respectively) from 12 luciferases revealed consensus sequence of C-x(3)-C-L-x(2)-L-x(4)-Cx(8)-P-x-R-C(X, amino acid residue) in both domains (Takenaka, Yamaguchi et al. 2012). Thus, highly conserved amino acid residues, C-x(3)-C-L-x(2)-L-x(4)-C-x(8)-P-x-R-C, which are present in both domains, were considered one of the criteria for the copepod luciferase (Takenaka, Yamaguchi et al. 2012). Because the similarity in the structure of the two domains was found in all of the copepod luciferases isolated in this study, the authors assumed this similarity in the structure of the two domains to be very characteristic of the luciferases isolated from Augaptiloidea species (Takenaka, Yamaguchi et al. 2012). Substitution of all cysteine residues with alanine in domains 1 and 2 of MpLuc1 resulted in complete loss of activity when it was expressed in Escherichia coli, suggesting an essential role of cysteine residues in luciferase activity (Takenaka, Yamaguchi et al. 2012). Thus, the presence of two domains with sequence similarity along with the highly conserved amino acid residues, C-x(3)-C-L-x(2)-L-x(4)-C-x(8)-P-x-R-C, which are present in both domains, can be used to identify any new luciferases isolated from Augaptiloidea species.

Gaussia luciferase in particular has been used as a secreted reporter in mammalian cells, reportedly providing increased assay sensitivity owing to its bright luminescence and accumulation in the cell culture medium. However, the light intensity decays rapidly under most conditions, thus requiring luminometers equipped with injectors to measure the transient peak luminescence. Mutants of GLuc have been described that provide Glow type luminescence, which may be suitable for HTS applications (Degeling, Bovenberg et al. 2013). Additionally, presence of 0.1% Triton X-100 in the assay buffer has been shown to stabilize Gaussia princeps luminescence resulting in sustained signal. Other methods of stabilizing the luminescent signal form GLuc and related luciferases are known in the art and have been described in the U.S. Pat. No. 7,939,286 B2.

Furthermore, the coelenterazine substrate is prone to chemical instability and high auto-luminescence background, properties that make sample handling difficult and decrease assay sensitivity. Although bright luminescence is generally desirable, a sustained signal with low background is necessary to enable efficient assay methods with high sensitivity. Preferably the luciferase should be small, monomeric, and structurally stable to environmental conditions. Recently, Promega engineered both an enzyme and substrate in combination to create a novel bioluminescence system capable of more efficient light emission with superior biochemical and physical characteristics (U.S. Pat. No. 8,557,970-B2) (Hall, Unch et al. 2012). Using a small luciferase subunit (19 kDa) from the deep sea shrimp Oplophorus gracilirostris, they improved luminescence expression in mammalian cells˜2.5 million-fold by merging optimization of protein structure with development of a novel imidazopyrazinone substrate (furimazine). The new luciferase, NanoLuc (NLuc), produces glow-type luminescence (signal half-life >2h) with a specific activity ˜150-fold greater than that of either firefly (Photinus pyralis) or Renilla luciferases similarly configured for glow-type assays. In mammalian cells, NanoLuc (NLuc) shows no evidence of posttranslational modifications or subcellular partitioning. The enzyme exhibits high physical stability, retaining activity with incubation up to 55° C. or in culture medium for >15 h at 37° C. Reporter quantitation is achievable even at very low expression levels to facilitate more reliable coupling with endogenous cellular processes.

As stated above, a major limitation of LDH release assay is its inability to distinguish between the death of target and effector cells. To overcome this limitation, the inventor engineered the target cells so that they express a reporter that is not expressed by the effector cells. In order to develop a sensitive assay of cell death, without being bound to a particular theory the inventor hypothesized that the reporter should ideally have the following properties: 1) it should be preferentially retained inside the cells and not secreted outside; 2) it should be relatively non-toxic to the host cells; 3) it should be relatively small in size so that it can readily leak outside when the integrity of the cell membrane is compromised; 4) it should be monomeric and not make aggregates inside the cells which could potentially prevent its leakage; 5) it should preferably not bound to any cellular proteins, which would prevent its leakage upon cell damage; 6) it should be relatively stable with relatively long half-life and not prone to degradation when released in the extracellular compartment; 7) it should be preferably resistant to proteases that are present in the serum and that are released during the process of cell death; 8) it should be preferably heat stable at 37° C.; 8) its activity should be readily measured by an assay which is quick, inexpensive, sensitive, homogenous (i.e. single step), with a broad linear range, and amenable to high-throughput screening applications; 9) if the reporter requires a substrate for the detection of its activity, then the substrate should not readily enter a live cell but should readily enter a dead or dying cell that has lost its cell membrane integrity. It is to be noted, however, that a reporter for the invention need not possess all the above properties.

In various embodiments, the reporter is a non-secretory form of a luciferase. In various embodiments, the non-secretory form of luciferase is obtained from copepods, deep sea shrimp, a beetle luciferase or homologs or orthologs thereof or mutants or functional variants or derivatives thereof. In various embodiments, the reporter is a non-secretory form of a thermostable alkaline phosphatase.

In exemplary embodiments, the copepods are selected from the group consisting of any one or more of Gaussia princeps, Pleuromamma abdominalis, Metridia pacifica, Metridia curticauda, Metridia asymmetrica, Metridia okhotensis, Metridia longa, Lucicutia ovaliformis, Heterorhabdus tanneri, and Pleuromamma scutullata.

In exemplary embodiments, the reporter luciferase is a non-secretory form of any one or more of GLuc, NanoLuc (NLuc), MLuc7, HtLuc, LoLuc, PaLuc1, PaLuc2, MpLuc1, McLuc1, MaLuc1, MoLuc1, MoLuc2, MLuc39, PsLuc1, LocLuc1-3, HtLuc2, TurboLuc16 (TLuc), Lucia-Luc, LucPPe-146-1H2, LucPPe-133-1B2, LucPPe-78-0B10, LucPPe49-7C6A, LucPpL-81-6G1, CBGRluc, Renilla (RLuc), Firefly Luc (Flue or FfLuc of fFLuc) or homologs or orthologs thereof or mutants or functional variants or derivatives thereof. All the reporters of the invention are designed to be expressed intracellularly and, therefore, lack a secretory signal. The DNA and Protein (PRT) SEQ IDs of several exemplary reporters of the invention, including luciferases, are provided in Table 1.

TABLE 1
Exemplary Reporters for use with in the compositions and methods described herein.

| SEQ ID (DNA) | SEQ ID (Prt) | REPORTER NAME |
|---|---|---|
| 1 | 31 | GLuc (Gaussia princeps Luc) |
| 2 | 32 | NLuc (NanoLuc) |
| 3 | 33 | TLuc (TurboLuc16) |
| 4 | 34 | MLuc7 (Metrida longa Luc7) M43L/M110L variant |
| 5 | 35 | LoLuc (Lucicutia ovaliformis Luc) |
| 6 | 36 | HtLuc (Heterorhabdus tanneri Luc) |
| 7 | 37 | PaLuc1 (Pleuromamma abdominalis Luc1) |
| 8 | 38 | PaLuc2 (Pleuromamma abdominalis Luc2) |
| 9 | 39 | MpLuc1[Metridia pacifica Luc1] |
| 10 | 40 | McLuc1 [Metridia curticauda Luc1] |
| 11 | 41 | MaLuc1 [Metridia asymmetrica Luc1] |
| 12 | 42 | MoLuc1 [Metridia okhotensis Luc1] |
| 13 | 43 | MoLuc2 [Metridia okhotensis Luc2] |
| 14 | 44 | MLuc39 [Metridia longa Luc39] |
| 15 | 45 | PsLuc1 [Pleuromamma scutullata Luc1] |
| 16 | 46 | LoLuc1-3 [Lucicutia ovaliformis Luc1-3] |
| 17 | 47 | HtLuc2 [Heterorhabdus tanneri Luc 2] |
| 18 | 48 | Lucia-Luc |
| 19 | 49 | RLuc (Renilla Luc) |
| 20 | 50 | Fluc or FfLuc (Firefly Luc) |
| 21 | 51 | LucPPe-146-1H2 |
| 22 | 52 | LucPPe-133-1B2 |
| 23 | 53 | LucPPe-78-0B10 |
| 24 | 54 | LucPPe49-7C6A |
| 25 | 55 | LucPpL-81-6G1 |
| 26 | 56 | CBGRluc |
| 27 | 57 | Embryonic Alkaline Phosphatase (EAP) |
| 28 | 58 | mCherry |
| 29 | 59 | EGFP (Enhanced Green Fluorescent Protein) |
| 30 | 60 | MYR-GGS-GLuc (M60L/M127L variant) |

In one embodiment, the reporter luciferase is GLuc (Gaussia princeps Luc) and comprises, consists of or consists essentially of the sequence set forth in SEQ ID NO: 1 or SEQ ID NO: 31 or a sequence with 70-99% homology to the sequence set forth in SEQ ID NO: 1 or SEQ ID NO: 31.

In another embodiment, the reporter luciferase is NLuc (NanoLuc) and comprises, consists of or consists essentially of the sequence set forth in SEQ ID NO: 2 or SEQ ID NO: 32 or a sequence with 70-99% homology to sequence set forth in SEQ ID NO: 2 or SEQ ID NO: 32.

In a further embodiment, the reporter luciferase is TLuc (TurboLuc16) and comprises, consists of or consists essentially of the sequence set forth in SEQ ID NO: 3 or SEQ ID NO: 33 or a sequence with 70-99% homology to the sequence set forth in SEQ ID NO: 3 or SEQ ID NO: 33.

In a further embodiment, the reporter luciferase is MLuc7-(Metrida longa Luc) M43L/M110L variant and comprises, consists of or consists essentially of the sequence set forth in SEQ ID NO: 4 or SEQ ID NO: 34 or a sequence with 70-99% homology to the sequence set forth in SEQ ID NO: 4 or SEQ ID NO: 34.

In another embodiment, the reporter luciferase is LoLuc (Lucicutia ovaliformis Luc) and comprises, consists of or consists essentially of the sequence set forth in SEQ ID NO: 5 or SEQ ID NO: 35 or a sequence with 70-99% homology to the sequence set forth in SEQ ID NO: 5 or SEQ ID NO: 35.

In another embodiment, the reporter luciferase is HtLuc (Heterorhabdus tanneri Luc) and comprises, consists of or consists essentially of the sequence set forth in SEQ ID NO: 6 or SEQ ID NO: 36 or a sequence with 70-99% homology to sequence set forth in SEQ ID NO: 6 or SEQ ID NO: 36.

In another embodiment, the reporter luciferase is PaLuc1 (Pleuromamma abdominalis Luc) and comprises, consists of or consists essentially of the sequence set forth in SEQ ID NO: 7 or SEQ ID NO: 37 or a sequence with 70-99% homology to the sequence set forth in SEQ ID NO: 7 or SEQ ID NO: 37.

In another embodiment, the reporter luciferase is PaLuc2 (Pleuromamma abdominalis Luc2) and comprises, consists of or consists essentially of the sequence set forth in SEQ ID NO: 8 or SEQ ID NO: 38 or a sequence with 70-99% homology to the sequence set forth in SEQ ID NO: 8 or SEQ ID NO: 38.

In another embodiment, the reporter luciferase is MpLuc1 (Metridia pacifica Luc1) and comprises, consists of or consists essentially of the sequence set forth in SEQ ID NO: 9 or SEQ ID NO: 39 or a sequence with 70-99% homology to sequence set forth in SEQ ID NO: 9 or SEQ ID NO: 39.

In another embodiment, the reporter luciferase is McLuc1 (Metridia curticauda Luc1) and comprises, consists of or consists essentially of the sequence set forth in SEQ ID NO: 10, or SEQ ID NO: 40 or a sequence with 70-99% homology to the sequence set forth in SEQ ID NO: 10 or SEQ ID NO: 40.

In another embodiment, the reporter luciferase is MaLuc1 (Metridia asymmetrica Luc1) and comprises, consists of or consists essentially of the sequence set forth in SEQ ID NO: 11 or SEQ ID NO: 41 or a sequence with 70-99% homology to sequence set forth in SEQ ID NO: 11 or SEQ ID NO: 41.

In another embodiment, the reporter luciferase is MoLuc1 (Metridia okhotensis Luc1) and comprises, consists of or consists essentially of the sequence set forth in SEQ ID NO: 12 or SEQ ID NO: 42 or a sequence with 70-99% homology to sequence set forth in SEQ ID NO: 12 or SEQ ID NO: 42.

In another embodiment, the reporter luciferase is MoLuc2 (Metridia okhotensis Luc2) and comprises, consists of or consists essentially of the sequence set forth in SEQ ID NO: 13 or SEQ ID NO: 43 or a sequence with 70-99% homology to sequence set forth in SEQ ID NO: 13 or SEQ ID NO: 43.

In another embodiment, the reporter luciferase is MLuc39 (Metridia longa Luc39) and comprises, consists of or consists essentially of the sequence set forth in SEQ ID NO: 14 or 44 or a sequence with 70-99% homology to sequence set forth in SEQ ID NO: 14 or SEQ ID NO: 44.

In another embodiment, the reporter luciferase is PsLuc1 (Pleuromamma scutullata Luc1) and comprises, consists of or consists essentially of the sequence set forth in SEQ ID NO:15 or SEQ ID NO: 45 or a sequence with 70-99% homology to sequence set forth in SEQ ID NO:15 or SEQ ID NO: 45.

In another embodiment, the reporter luciferase is LoLuc1-3 (Lucicutia ovaliformis Luc1-3) and comprises, consists of or consists essentially of the sequence set forth in SEQ ID NO: 16 or SEQ ID NO: 46 or a sequence with 70-99% homology to sequence set forth in SEQ ID NO:16 or SEQ ID NO: 46.

In another embodiment, the reporter luciferase is HtLuc2 (Heterorhabdus tanneri Luc2) and comprises, consists of or consists essentially of the sequence set forth in SEQ ID NO: 17 or SEQ ID NO: 47 or a sequence with 70-99% homology to the sequence set forth in SEQ ID NO:17 or SEQ ID NO: 47.

In another embodiment, the reporter luciferase is Lucia-Luc and comprises, consists of or consists essentially of the sequence set forth in SEQ ID NO: 18 or SEQ ID NO: 48 or a sequence with 70-99% homology to sequence set forth in SEQ ID NO: 18 or SEQ ID NO: 48.

In another embodiment, the reporter luciferase is Renilla Luc (RLuc) and comprises, consists of or consists essentially of the sequence set forth in SEQ ID NO: 19 or SEQ ID NO: 49 or a sequence with 70-99% homology to the sequence set forth in SEQ ID NO: 19 or SEQ ID NO: 49.

In another embodiment, the reporter luciferase is Firefly Luc (FLuc or FfLuc or fFLuc) and comprises, consists of or consists essentially of the sequence set forth in SEQ ID NO: 20 or SEQ ID NO: 50 or a sequence with 70-99% homology to the sequence set forth in SEQ ID NO: 20 or SEQ ID NO: 50.

In another embodiment, the reporter luciferase is LucPPe-146-1H2 and comprises, consists of or consists essentially of the sequence set forth in SEQ ID NO: 21 or SEQ ID NO: 51 or a sequence with 70-99% homology to the sequence set forth in SEQ ID NO: 21 or SEQ ID NO: 51.

In another embodiment, the reporter luciferase is LucPPe-133-1B2 and comprises, consists of or consists essentially of the sequence set forth in SEQ ID NO: 22 or SEQ ID NO: 52 or a sequence with 70-99% homology to the sequence set forth in SEQ ID NO: 22 or SEQ ID NO: 52.

In another embodiment, the reporter luciferase is LucPPe-78-0B10 and comprises, consists of or consists essentially of the sequence set forth in SEQ ID NO: 23 or SEQ ID NO: 53 or a sequence with 70-99% homology to the sequence set forth in SEQ ID NO: 23 or SEQ ID NO: 53.

In another embodiment, the reporter luciferase is LucPPe49-7C6A and comprises, consists of or consists essentially of the sequence set forth in SEQ ID NO: 24 or SEQ ID NO: 54 or a sequence with 70-99% homology to the sequence set forth in SEQ ID NO: 24 or SEQ ID NO: 54.

In another embodiment, the reporter luciferase is LucPpL-81-6G1 and comprises, consists of or consists essentially of the sequence set forth in SEQ ID NO: 25 or SEQ ID NO: 55 or a sequence with 70-99% homology to the sequence set forth in SEQ ID NO: 25 or SEQ ID NO: 55.

In another embodiment, the reporter luciferase is CBGRluc and comprises, consists of or consists essentially of the sequence set forth in SEQ ID NO: 26 or SEQ ID NO: 56 or a sequence with 70-99% homology to the sequence set forth in SEQ ID NO: 26 or SEQ ID NO: 56.

In another embodiment, the reporter is a thermostable alkaline phosphatase (e.g., Embryonic Alkaline Phosphatase) and comprises, consists of or consists essentially of the sequence set forth in SEQ ID NO: 27 or SEQ ID NO: 57 or a sequence with 70-99% homology to the sequence set forth in SEQ ID NO: 27 or SEQ ID NO: 57.

In another embodiment, the reporter fluorescent protein is mCherry and comprises, consists of or consists essentially of the sequence set forth in SEQ ID NO: 28 or SEQ ID NO: 58 or a sequence with 70-99% homology to the sequence set forth in SEQ ID NO: 28 or SEQ ID NO: 58.

In another embodiment, the reporter fluorescent protein is EGFP and comprises, consists of or consists essentially of the sequence set forth in SEQ ID NO:29 or SEQ ID NO: 59 or a sequence with 70-99% homology to the sequence set forth in SEQ ID NO: 29 or SEQ ID NO: 59.

In another embodiment, the reporter luciferase is GLuc (M60L/M127L) variant and comprises, consists of or consists essentially of the luciferase sequence set forth in SEQ ID NO:30 or SEQ ID NO: 60 or a sequence with 70-99% homology to the luciferase sequence set forth in SEQ ID NO: 30 or SEQ ID NO: 60.

The present invention provides methods of assaying cytotoxicity in vitro and in vivo. The methods are non-radioactive and do not require labeling the cells prior to each assay, and thus are easier and safer to perform than assays which require labeling cells prior to each assay. The methods provided can help distinguish between the death of target and effector cells and are highly sensitive with broader linearity than standard cytotoxicity assays, allowing minor differences in the cytotoxicity kinetics of different products to be detected. Further, in some embodiments the simplicity of the methods provided herein means that they are homogenous and faster than standard cytotoxicity assays, and also have the potential to be optimized for high-throughput screening to identify compounds that have cytotoxic activity of their own or can modulate (i.e. increase or decrease) the cytotoxic activity of different agents. The methods can be used to detect the cytotoxic activity of different drugs, small molecules, biologicals (e.g. antibodies), and cells, including lymphocytes and NK cells and immune cells that have been modified to express chimeric and synthetic receptors and T cell receptors. The methods also can be performed on both adherent cells and cells in suspension. The methods can be performed on both eukaryotic and prokaryotic cells and both in vitro and in vivo. The methods can be performed on primary cells as well as cell lines. The method can be performed on normal cells or diseased cells or disease-associated cells, such as cancer cells or cells infected with infectious organisms. The method can be also adapted to detect the toxicity of different agents on vital organs in vivo. In addition, the methods can be adapted for multiplexing so that cytotoxic activity of a therapeutic agent on different cell types (i.e. normal vs. cancer; liver cancer vs. lung cancer etc.) and different organs (e.g. liver vs. kidney) can be measured in the same assay and in the same animal, respectively.

The method can be used to detect the cytotoxicity of any agent targeting any molecule or antigens expressed in a cell. In some embodiments, the methods can be used to measure cytotoxicity of one or more agents that target one or more of the antigens selected from a group consisting of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRviii); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); FmsLike Tyrosine Kinase 3 (FLT3); Tumor-associated glycoprotein 72 (TAG72); CD38; CD44v6; a glycosylated CD43 epitope expersed on acute leukemia or lymphoma but not on hematopoietic progenitors, a glycosylated CD43 epitope expressed on non-hematopoietic cancers, Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-llRa); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDClalp(1-4)bDGlcp(1-1)Cer); transglutaminase 5 (TGS5); high molecular weight-melanoma associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCT A-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B 1 (CYP1B 1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1), MPL, Biotin, c-MYC epitope Tag, CD34, LAMP1 TROP2, GFRalpha4, CDH17, CDH6, NYBR1, CDH19, CD200R, Slea (CA19.9; Sialyl Lewis Antigen) Fucosyl-GM1, PTK7, gpNMB, CDH1-CD324, DLL3, CD276/B7H3, IL11Ra, IL13Ra2, CD179b-IGLll, ALK TCRgamma-delta, NKG2D, CD32 (FCGR2A), Tn ag, CSPG4-HMW-MAA, Tim1-/HVCR1, CSF2RA (GM-CSFR-alpha), TGFbetaR2, VEGFR2/KDR, Lews Ag, TCR-beta1 chain, TCR-beta2 chain, TCR-gamma chain, TCR-delta chain, FITC, Leutenizing hormone receptor (LHR), Follicle stimulating hormone receptor (FSHR), Chorionic Gonadotropin Hormone receptor (CGHR), CCR4, GD3, SLAMF6, SLAMF4, HIV1 envelope glycoprotein, HTLV1-Tax, CMV pp65, EBV-EBNA3c, influenza A hemagglutinin (HA), GAD, PDL1, Guanylyl cyclase C (GCC), KSHV-K8.1 protein, KSHV-gH protein, HLA, HLA-A, HLA-A2, HLA-B, HLA-C, HLA-DP, HLA-DM, HLA-DOA, HLA-DOB, HLA-DQ, HLA-DR or HLA-G.

The method is based on the principle that cell membrane integrity is lost upon cell death. In one embodiment, provided herein are assays using any reporter that can be expressed intracellularly and the measure of whose activity is increased upon cell damage. The increase in the measured activity of the reporter upon cell damage can happen due the release or leakage of the reporter into the extracellular compartment where it can readily interact with its substrate. Alternatively, the increase in the increase in the measured activity of the reporter upon cell damage can happen due to loss of cell membrane integrity that results in the increased or faster penetration of the substrate into dead and dying cells where it can readily interact with the reporter still trapped inside the cell. Finally, both the above processes may contribute to the increase in the reporter activity upon cell damage.

In some embodiments, the assays include the steps of i) providing a target cell expressing non-secretory form of a reporter; ii) exposing the target cell to a test agent capable of inducing cytotoxicity and a control agent not capable of inducing cytotoxicity; iii) exposing the target cell from (ii) or the medium (i.e. supernatant) from target cell from (ii) or the target cell with the medium from (ii) to a reporter-specific substrate; iv) assaying the reporter-specific activity; and v) comparing the reporter-specific activity between the test agent- and the control agent-treated samples. In some embodiments, an increase in the reporter-specific activity in the test agent-treated samples as compared to the control agent-treated samples is indicative of the test agent being cytotoxic to the target cell In some embodiments, an increase in reporter activity relative to a reference value is indicative of the agent being cytotoxic to the target cell.

In some embodiments, the reference value is the reporter activity in any one or more of (i) mean or median reporter activity in target cells that do not express the reporter, (ii) mean or median reporter activity in target cells that express reporter but are not treated with the test agent(s); (iii) mean or median reported activity in target cells that are left untreated; (iv) mean or median reporter activity in target cells that are not treated with the substrate for the reporter, or (iii) a combination thereof. In some embodiments, a change in reporter activity is an increase in reporter activity relative to the reference value. In some embodiments, reporter activity is increased relative to the reference value by at least or about 10%, 20%, 30%, 40%, 50%, 60%, 70%, 80%, 90% or 100%. In some embodiments, reporter activity is increased relative to the reference value by at least or about 1-fold, 2-fold, 3-fold, 4-fold, 5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold or a combination thereof.

In some embodiments, the reporter is non-secretory form of a reporter that is stable under the culture conditions. The preferred reporter for the cytotoxicity assay is stable under the conditions of the cytotoxicity assay. In the preferred embodiment, the reporter is stable under the culture conditions of the cytotoxicity assay for more than 20 minutes; for more than 30 minutes; for more than 1 hour; for more than 2 hours; for more than 4 hours; for more than 6 hours; for more than 12 hour; for more than 24 hours; preferably for more than 48 hours; or more preferably for more than 96 hours at 37° C. In some embodiments, the reporter is non-secretory form of luciferase, preferentially a thermostable luciferase, for example, GLuc, NLuc, MLuc7, Lucia-Luc, PaLuc1, TurboLuc16 (TLuc), Renilla, thermostable luciferases from Photuris pennsylvanica and Pyrophorus plagiothalamus (as described in PCT/US99/30925) or any of their functional variants or homologs. In the preferred embodiments, the reporter is non-secretory form of a reporter whose activity is not negatively affected by cellular processes activated before, during or after induction of cell death, such as activation of cell death proteases or change in redox potential.

In some embodiments, the target cells express one reporter. In an alternate embodiment, the target cells express more than one reporter. In some embodiment, the two reporters belong to the same class, e.g. two marine luciferases. In other embodiments, the two reporters belong to different classes, for example a marine luciferase (e.g. NLuc) and a beetle luciferase (e.g. LucPPe-146-1H2) or a marine luciferase and an embryonic alkaline phosphatase.

In various embodiments, vectors that may express the reporter include but are not limited to plasmid vectors (e.g. pCDNA3), retroviral vector (e.g. MSCVpac, MSCVneo, MSCVhygro), lentiviral vectors (e.g. pLenti-EF1α), adenoviral vectors, adenoassociated viral vector, sleeping beauty transposon, piggyback transposon. In other embodiment, the reporter can be introduced in the cells without the use of a vector, such as by transfection of DNA, RNA or protein encoding the reporter. Various methods of introducing DNA, RNA or protein inside cells have been described in the literature, including lipofection, electroporation, nucleofection and by causing transient perturbation in cell membrane (US2012/060646 and WO 2013/059343) (Sharel, Adamo et al.) In some embodiments, the target cell can be any one or more of healthy cells, cancer cells, infected cells (for example, infected by virus, bacteria, and/or parasites). In some embodiments, the agent that results in cellular cytotoxicity is an effector cell. In exemplary embodiments, effector cells may be any one or more of peripheral blood mononuclear cells (PBMCs), natural killer (NK) cells, monocytes, T cells, cytotoxic T cells, recombinant (for example, expressing CARs or TCR) T cells, neutrophils or combinations thereof.

In some embodiments, the assays described herein may be used to screen a candidate antibody for its ability to induce antibody-dependent cellular cytotoxicity (ADCC) against target cells. In some embodiments, the assays described herein may be used to identify a patient having a disease associated with target cells that is suitable for treatment with a candidate antibody. In some embodiments, the assays described herein may be used to screen for compounds for the ability to modulate ADCC. In some embodiments, the assays described herein may be used to determine the optimal concentration of an antibody to induce an ADCC response in a subject.

In some embodiments, the assays described herein may be used to test the effects of an agent, for example, T cells expressing exogenous T cell receptor, or chimeric and synthetic receptors. The assay may be also used to test the effect of bispecific antibodies, BiTEs, DART (dual affinity retargeting) monoclonal antibodies (mAbs), non-immunoglobulin binding scaffolds (e.g. affibodies, DARINS etc), radiolabelled antibodies and/or antibody drug conjugates on target cells (for example, in drug discovery).

In some embodiments, the assays described herein may be used to carry out high throughput screening for agents, such as chemical compounds including small molecule compounds, biological and cytokines, that can modulate (enhance or decrease) the function of other agents, such as CARs, TCRs, biologicals (e.g. TNF, Interferons), bispecific antibodies (including BiTE), DART (Dual affinity retargeting), monoclonal antibodies and antibody-drug conjugates (ADCs), when used alone or in combination.

In some embodiments, the assays described herein may be used in quality control as potency assays, for example, at the site of production of a cellular therapy product. For example, CARs targeting CD19 antigen for the treatment of acute and chronic lymphocytic leukemias, lymphoma and myeloma may need to be developed for each patient individually and need to be tested at the time of manufacturing to ensure consistency and reliable performance. Potency assays are mandated by US Congress before a cell therapy product, such as CAR-T, can be released for administration and are an FDA-requirement for product release. Matador cytotoxicity assays based on RAJI-GLuc, RAJI-NLuc and RAJI-MLuc7 cell lines, as described herein, may be used as potency assays to ensure the quality of the CAR product since the assays described herein are extremely sensitive (down to a single cell level), quick, non-radioactive and easy to use. In an embodiment, the assay is performed on CAR-T cell products being manufactured on different days of manufacturing to monitor the potency of the product. In a standard cytotoxicity assay, target cells are co-cultured with the effector cells for 4 hours to 96 hours. The extreme sensitivity of the Matador assay allows shortening of the co-culture period to an hour or less. This would result in significant time-saving in the CAR-T cell manufacturing and product-release process, and hence carries significant commercial value.

The inventor has shown that the Matador assays can be performed on primary patient samples. The assays described herein have the advantage of directly measuring the activity of the cell therapy product on patient's cancer cells and assessing the sensitivity of patient's sample to the therapeutic product. For example, even though the therapeutic product (for example, CAR-T cell) may be effective and functional in a cell line model, in rare cases a patient's leukemia cells may still not respond to the therapeutic product because the leukemia cells express inhibitory receptors that block the function of the therapeutic product (for example, CAR modified T cells). In some embodiments, the assays described herein may be used to test the efficacy of other cellular therapy and biological products, including but not limited to NK cells, donor lymphocytes, T cells and their subsets, microtransplantation, monoclonal antibodies, antibody drug conjugates and bispecific antibodies (including BiTE), when used alone or combinations thereof.

The CAR-T cells are living drugs as the cells once infused can multiply in the patient. They can also kill a target cell that expresses as few as 100 molecules of their target antigen. In addition, CAR cells can kill healthy cells that do not express their target via a bystander effect through production of cytokines. Therefore, toxicity of the CAR and other cellular therapy products (including antibody drug conjugates, and Bispecific T cell engagers or BiTE, DART) on normal tissues is a major concern as even low level expression of the CAR target-antigen on a vital organ can be lethal. The assay described herein is sensitive (down to a single cell level) and provides a useful platform to monitor and assure the safety of each lot of CAR-T cell product that is generated. For example, a panel of cell lines of different tissue origins expressing GLuc (or NLuc or any of the other reporter of the invention) can be used to rapidly test for killing by each lot of the CAR-T cell product, which would be helpful in predicting their toxicity on normal tissues. In some embodiments, the panel of primary cells or cell lines to be used in the Matador assay may express different levels of the target antigen. In some embodiments, the panel of primary cells or cell lines to be used in the Matador assay may be selected to have lost a particular target antigen so as to determine the off-target toxicity of the test product (e.g., a CAR-T cell).

In various embodiments, in vitro and in vivo applications of the assay are described and include: 1) Immuno-binding the reporter (e.g., GLuc) by an antibody as has been described (Bovenberg, Degeling et al. 2012) and then measuring the activity of the reporter. This may be useful in case of interfering substances in the media or blood or serum that can interfere with the assay for the reporter; 2) Epitope tagging the reporter (e.g. GLuc, NLuc, TurboLuc or MLuc7 or any of the other reporters) with tags such as Flag, HA, His, Myc, V5, AcV5 or Glu (van Rijn, Nilsson et al. 2013, van Rijn, Wurdinger et al. 2014) and then using such tagged reporter constructs to generate target cells ectopically expressing the reporter or reporters; 3) It is also possible to use the reporters in combination. For example, it is possible to use GLuc in combination with a thermostable beetle luciferase (e.g. LucPPe-146-1H2) as they have different substrates. As an example, RAJI cells (CD19⁺) stably expressing GLuc are mixed with HL60 cells (CD19⁻) stably expressing LucPPe-146-1H2 in a 1:1 ratio and are co-cultured for 4 hours with CD19 CAR-T cells. At the end of the co-culture period, the cell supernatant is tested for GLuc activity using coelentrazine as its substrate and for LucPPe-146-1H2 activity using D-luciferin as a substrate to determine the relative cytotoxicity of the CD19 CAR-T cells against RAJI and HL60 cells, respectively. It is also possible to use GLuc in combination with NLuc, TurboLuc16, MLuc7 or other reporters. As an example, RAJI cells (CD19⁺) stably expressing GLuc-Flag are mixed with HL60 cells (CD19⁻) stably expressing NLuc-Myc in a 1:1 ratio and are co-cultured with CD19 CAR-T cells. At the end of the co-culture period, the cell supernatant is collected and GLuc-Flag and NLuc-Myc proteins are separated by immunobinding with Flag and Myc antibody containing beads or control beads. Subsequently, the GLuc and NLuc activities are measured on the Flag and Myc antibody-bound fractions using coelentrazine as a substrate.GLucGLucGLucGLucGLucGLuc; 4) using mutants of GLuc and NLuc that emit light at different wavelengths; 5) having the target cells express one reporter (e.g., GLuc) and the effector cells (e.g., NK or T cells) express a different reporter (e.g. LucPPe-146-1H2NLuc-Myc) and measuring the cell death of both target and effector cells from the same experiment by measuring GLuc and LucPPe-146-1H2 activities.GLuc; 6) monitoring the susceptibility of leukemic and cancer cells to cytotoxic cells derived from different allogeneic donors. This may include infecting the leukemic or cancer cells with a viral vector expressing the appropriate reporter construct or by transient transfection with reporter plasmid or in vitro transcribed RNA and then exposing the reporter-expressing cells to cytotoxic cells (NK, T cells, peripheral blood mononuclear cells) from different donors to identify the donor most suitable for the patient; 7) performing in vivo assay by xenografting the reporter cell line expressing the reporter (e.g., GLuc or LucPPe-146-1H2) in an animal and treating the animal with appropriate agent (drug, irradiation or cytotoxic effector cell) and then measuring the released GLuc or LucPPe-146-1H2 activity in blood, urine, plasma, serum, CSF or other body fluids directly or after immunobinding using assay described above and known in the art; 8) the above method can be also adapted for use in cells of other origin such as bacterial, yeast and insect cells and then used for screening drugs/compounds that exert cytotoxicity or inhibit cytotoxicity on these cells.

Currently, there is no easy, sensitive and inexpensive way to monitor toxicity of different drugs or interventions on different organs. The Matador assay described herein can be adapted for generating animals that can be used for quickly detecting toxicity of different agents or to monitor efficacy of different interventions against diseases (e.g., Alzheimer, stroke, ischemia), which are accompanied by cellular toxicity and injury. In an exemplary embodiment, transgenic animals can be generated that express GLuc constitutively or in a tissue-specific manner by using tissue- or cell lineage-specific promoters. The animals can be exposed to a candidate agent (e.g. a new drug or a CAR-T cell product) and toxicity of agent monitored by assaying for GLuc activity in body fluids (e.g., serum, blood, plasma, urine, CSF, ascites fluid etc.) either directly or by including the step of immunobinding to purify GLuc from the body fluid.

In another embodiment, transgenic animals can be generated that express a reporter, such as GLuc, with different tags in different cellular compartments, lineages or organs using methodologies known in the art. Thus, it is possible to generate transgenic animals expressing GLuc-Flag in liver, GLuc-HA in heart, GLuc-V5 in kidneys, and GLuc-AcV5 in gastrointestinal tract. Using the techniques known in the art, it is possible to generate single, double, triple or multiple transgenic animals expressing the above constructs singly or in combination. It is also possible to generate transgenic animals expressing different reporter (e.g, GLuc or NLuc) constructs in different lineages or compartments of a single organ (e.g. proximal and distal tubules of kidney or different cellular compartments of gut or CNS or heart or liver, or lungs.). Such single or multiple transgenic animals can be exposed to a candidate test agent and its toxicity measured by checking for the reporter activity in the body fluid either directly or after immunobinding using antibodies against different epitope tags that could be linked to the reporters or using antibodies against different reporters (e.g. antibodies against GLuc or NLuc).

To increase the half-life of the reporters of the invention in the blood and other body fluids, it is possible to generate their fusion variants by adding the sequence encoding albumin or an albumin binding domain (ABD) to their coding region. The fusion proteins of the reporters with albumin (e.g. GLuc-Alb) or with ABD (e.g. GLuc-ABD) are expected to have longer half lives as they will not be filtered through the urine. In an exemplary embodiment, cell lines can be generated expressing GLuc-Alb or GLuc-ABD or GLuc-ABD-Flag and used in Matador assay in vivo. It is also possible to increase the half-life of a reporter by fusing it (with or without via a flexible Glycine-serine linker) to an antibody or antibody fragment (e.g. scFV) or a single domain antibody (e.g. nanobody) directed against albumin. An example of a nanobody against albumin is provided in patent WO 2011/144749 A1.

In another embodiment, transgenic animals expressing the reporters of the invention can be used to measure the efficacy of an agent or endeavor to reduce cell toxicity or cell death, such as in the case of Alzheimer disease, stroke, degenerative disease, or autoimmune diseases. In an exemplary embodiment, transgenic animals expressing GLuc in neurons are generated using neuron-specific Thy1 or Nestin promoter. Such animals are used to test the efficacy of a candidate anti-stroke agent in an appropriate stroke model known in the art. In case the test agent is effective, it will reduce the death of neurons and the release of GLuc from them. The GLuc activity, in turn, can be measured ex vivo in blood, urine or CSF or by in vivo bioluminescence imaging following injection of coelentrazine.

In another embodiment, the reporters of the invention are expressed in different cells of an animal using viral vectors (e.g. AAV, Adeno, lentiviral etc.) and then these animals are used for monitoring cytotoxicity.

### Kits

In various embodiments, the present invention provides a kit for assessing cytotoxicity of an agent. In various embodiments, the kits comprise vectors, target cells, primary cells, agents of interest or combinations thereof and instructions for carrying out the assays.

The kit is an assemblage of materials or components, including at least one of the inventive reporters, substrates, buffers, vectors and cell lines. Thus, in some embodiments the kit contains a cell or a cell line expressing non-secretory forms of a reporter (for example, GLuc) and the substrate for the reporter. For kits of the invention the reporter(s), the reporter substrate(s), and buffers and other chemicals can each be contained in a separate container, or they can be contained in a single container. The kits can also optionally comprise a second substrate or a quenching agent for a luminescent enzyme reaction.

The exact nature of the components configured in the inventive kit depends on its intended purpose. In one embodiment, the kit is configured particularly for research and development. In another embodiment, the kit is configured particularly for quality control testing or potency testing in manufacturing of a drug, antibody, biological or cellular therapy product. In yet another embodiment, the kit is configured for testing human samples. In further embodiments, the kit is configured for veterinary applications.

Instructions for use may be included in the kit. “Instructions for use” typically include a tangible expression describing the technique to be employed in using the components of the kit to effect a desired outcome, such as to test the cytotoxicity of a CAR-T cell product. Still in accordance with the present invention, “instructions for use” may include a tangible expression describing the preparation of components for use with the assays described herein. Optionally, the kit also contains other useful components, such as, measuring tools, diluents, buffers reporter substrates or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a glass vial used to contain suitable quantities of a composition containing a volume of the non-secretory form of GLuc encoding vector. The packaging material generally has an external label which indicates the contents and/or purpose of the kit and/or its components

### EXAMPLES

### Example 1

### Expression Vectors

The pcDNA3 mammalian expression vector was obtained from Invitrogen, ThermoFisher Scientific. The pLENTI-Blast vector was derived from pLenti6v5gw_lacz vector (Invitrogen; ThermoFisher Scientific) by removal of the LacZ gene. The expression of the inserted gene in this vector is driven by CMV promoter. pLenti-MP2 was a gift from Pantelis Tsoulfas (Addgene plasmid #36097) and was used to generate pLENTI-EF1α lentiviral vector (SEQ ID NO: 91) by replacement of the CMV promoter with human EF1α promoter using standard molecular biology techniques. psPAX2 was a gift from Didier Trono (Addgene plasmid #12260). The pLP/VSVG envelope plasmid and 293FT cells were obtained from Invitrogen (ThermoFisher Scientific). The retroviral transfer vector MSCVneo, MSCVhygro, and MSCVpac and the packaging vector pKAT were obtained from Dr. Robert Illaria's laboratory. phRGTK Renilla Luciferase plasmid was from Promega. To construct lentiviral constructs encoding FMC63-BBZ Chimeric antigen receptor (CAR), the gene fragment encoding FMC63 CAR was synthesized by GeneArt™ using the published sequence as template. The gene fragment was then used as a template in a PCR reaction to amplify the encoded sequence which was then cloned in a lentiviral vector driven by an EF1α promoter promoter and in frame with T2A ribosomal skip sequence followed by EGFP nucleotide sequence using standard molecular biology techniques. The resulting construct was labeled pLenti-EF1α-FMC63-MYC-BBZ-T2A-EGFP. The DNA and protein sequences of this CAR insert are provided in SEQ ID NO: 79 and SEQ ID NO: 85, respectively. A lentiviral construct, pLenti-EF1α-FMC63-MYC-BBZ-T2A-PAC, was also constructed by replacement of EGFP gene with a Puromycin resistance gene. The DNA and protein sequences of this CAR insert are provided in SEQ ID NO: 80 and SEQ ID NO: 86, respectively. Finally, a pLenti-EF1α-FMC63-MYC-CD28Z-T2A-Pac construct was made by using a synthetic CD28Z gene fragment (synthesized by IDT) as a template for PCR amplification and standard molecular biology techniques to replace the BBZ cassette with CD28Z cassette. The DNA and protein sequences of this CAR insert are provided in SEQ ID NO: 81 and SEQ ID NO: 87, respectively. CAR constructs targeting other antigens (e.g., CD22, MPL etc.) were generated similarly by replacing the nucleotide sequence encoding the FMC63 scFV with the nucleotide sequence encoding scFVs targeting the different antigens.

All the luciferase constructs used in this invention were designed to lack a secretory signal. The nucleotide sequences encoding NLuc, MLuc7-MM-LL, TurboLuc16, PaLuc1, Lucicutia ovaliformis luciferase, Heterorhabdus tanneri luciferase, Lucia-luciferase and LucPPe-146-1H2 were codon optimized using GeneArt™ software (Thermo Fisher Scientific) and gene-fragments encoding the optimized sequences were synthesized by GeneArt™ or Integrated DNA Technologies (IDT). The gene fragments were used as template in PCR reactions using custom primers to amplify the corresponding DNAs (lacking the secretory signal), which were then cloned in different expression vectors using standard molecular biology techniques. In all cases, where the nucleotide sequence containing the secretory signal was deleted, an ATG start codon was included in the 5′ custom primer that was used to amplify the DNAs. In some cases, the amplified fragments were cloned in-frame with nucleotide sequences encoding different epitope tags, such as FLAG (SEQ ID NO: 61), 3×FLAG (SEQ ID NO: 62), AcV5 (SEQ ID NO: 64), MYC (SEQ ID NO: 65) and HA (SEQ ID NO: 63). Essentially a similar strategy was used to clone codon optimized GLuc (also called hGLuc), Fire-Fly luciferase (FLUC or FfLuc) and Renilla Luciferase except the plasmids encoding the corresponding cDNAs were used as template in PCR reactions. Unless indicated otherwise, all luciferases (e.g., GLuc, NLuc, TLuc, PaLuc, Lucia-Luc etc.) described in this invention were used lacking their signal peptide or the secretory signal to target their expression to the cytosolic compartment. The sequences of several exemplary vectors encoding the different luciferases are provided in SEQ ID NO: 92 to SEQ ID NO: 100. These vectors can be used to clone any new reporter by replacing the existing reporter inserts using standard molecular biology techniques.

The sequence of inserts was confirmed by automated sequencing. The DNA and protein SEQ ID NOs of the different reporters used in this invention are shown in Tables 1. Table 2 provides the characteristics of exemplary expression constructs used in this invention.

TABLE 2
Characteristics of Exemplary Expression Constructs

| | | | | Antibiotic for |
|---|---|---|---|---|
| | | | | Selection of |
| | | | | mammalian |
| | Construct Name | Vector Backbone | Promoter | cells |
| 1 | pLenti-EF1a (or pLenti-EF1α) | pLenti-EF1a | EF1a | None |
| 2 | pLENTI-NLuc-AcV5-Blasticidin-Pa08 | pLenti6v5gw_lacz | CMV | Blasticidin |
| 3 | pLENTI-TurboLuc-16-X3Flag-Blast-C04 | pLenti6v5gw_lacz | CMV | Blasticidin |
| 4 | pLenti-EF1a-Pac-T2A-GLuc-B07 | pLenti-EF1a | CMV | Puromycin |
| 5 | MSCVhygro-GLuc-HA-G02 | MSCVhygro | MSCV LTR | G418 |
| 6 | MSCVpac-GLuc-HA-R03 | MSCVpac | MSCV LTR | Puromycin |
| 7 | MSCVneo-Lucia-Luc-x3-Flag-B08 | MSCVneo | MSCV LTR | G418 |
| 8 | pLENTI-GLuc-Flag-blast-B07 | pLenti6v5gw_lacz | CMV | Blasticidin |
| 9 | MSCVneo-GLuc-X3-FLAG-Q02 | MSCVneo | MSCV LTR | G418 |
| 10 | MSCVhygro-MLuc7-MM-LL-HA-R03 | MSCVhygro | MSCV LTR | Hygromycin |
| 11 | MSCVhygro-Renilla-V03 | MSCVhygro | MSCV LTR | Hygromycin |
| 12 | MSCV-hygro-NLuc-AcV5-D07 | MSCVhygro | MSCV LTR | Hygromycin |
| 13 | pLENTI-EF1a-FfLuc-Blasticidin-A07 | pLenti6v5gw_lacz | EF1a | Blasticidin |
| 14 | Plenti-MLuc7-MM-LL-HA-C03 | pLenti6v5gw_lacz | CMV | Blasticidin |
| 15 | MSCVhygro-2xFLAG-L-ovaliformis-AcV5- | MSCVhygro | MSCV LTR | Hygromycin |
| | D04 | | | |
| 16 | MSCV-neo-NLuc-AcV5-B04 | MSCVneo | MSCV LTR | G418 |
| 17 | MSCVpac-Nluc-AcV5-C08 | MSCVpac | MSCV LTR | Puromycin |
| 18 | pLenti-H-tanneri-Luc-x3-Flag-T05 | pLenti6v5gw_lacz | CMV | Blasticidin |
| 19 | MSCVhygro-H-tanneri-Luc-Flag-U02 | MSCVhygro | MSCV LTR | Hygromycin |
| 20 | pLenti-2XFLAG-L-ovaliformis-AcV5-S01 | pLenti6v5gw_lacz | CMV | Blasticidin |
| 21 | pLenti-LucPPe-146-1H2-3X-FLAG-S01 | pLenti6v5gw_lacz | CMV | Blasticidin |
| 22 | pLenti-EF1-LucPPe-146-1H2-Xho-Flag-Pac- | pLenti-EF1a | EF1a | Puromycin |
| | R01 | | | |
| 23 | MSCVhygro-PaLuc1-x3Flag-C01 | MSCVhygro | MSCV LTR | Hygromycin |
| 24 | pCDNA3 | pCDNA3 | CMV | G418 |

### Lentivirus and Retrovirus Viruses

Lentiviruses were generated by calcium phosphate based transfection in 293FT cells essentially as described previously (Matta, Hozayev et al. 2003). Briefly, 293FT cells were grown in DMEM with 10% FCS 4 mM L-Glutamine, 0.1 mM MEM Non-Essential Amino Acids, and 1 mM MEM Sodium Pyruvate (hereby referred to as DMEM-10). For generation of lentiviruses, 293FT cells were plated in 10 ml of DMEM-10% FCS medium without antibiotics in a 10 cm tissue culture plate so that they will be approximately 80 confluent on the day of transfection. The following day, the cells were transfected by calcium phosphate transfection method using 10 g of lentiviral expression plasmid encoding different genes, 7.5 g of PSPAX2 plasmid and 2 g of PLP/VSVG plasmid. Approximately 15-16 hours post-transfection, 9 ml of media was removed and replaced with 5 ml of fresh media. Approximately, 48 hours post-transfection, 5 ml of supernatant was collected (first collection) and replaced with fresh 5 ml media. Approximately 72 hours post-transfection, all media was collected (second collection, usually around 6 ml). The collected supernatants were pooled and centrifuged at 1000 rpm for 1 minute to remove any cell debris and non-adherent cells. The cell-free supernatant was filtered through 0.45 m syringe filter. In some cases, the supernatant was further concentrated by ultra-centrifugation at 18500 rpm for 2 hours at 4° C. The viral pellet was re-suspended in 1/10 of the initial volume. The virus was either used fresh to infect the target cells or stored frozen in aliquots at −80° C. In general, primary cells (e.g. T cells) were infected using spin-infection (1800 rpm for 90 minutes at 37° C. with 300 μl of concentrated virus in the presence of 8 μg/ml of Polybrene® (Sigma, Catalog no. H9268) in the morning. The media was changed in the evening and the infection was repeated for two more days for a total of 3 infections. After the 3^{rd} infection, the cells were pelleted and resuspended in the media with respective antibiotics and place in the cell culture flask for selection, unless indicated otherwise. In cases, where cells were infected with a lentivirus expressing EGFP, they were expanded without drug-selection or flow-sorted to enrich for EGFP-expressing cells. For infection of cancer cell lines, approximately 500,000 cells were infected with 2 ml of the un-concentrated viral supernatant in a total volume of 3 ml with Polybrene® (Sigma, Catalog no. H9268). Then next morning, the cells were pelleted and resuspended in the media with respective antibiotics and place in the cell culture flask for selection.

Essentially a similar procedure as described above for lentivirus vector production was used for generation of retroviral vectors with the exception that 293FT cells were generally transfected in 10 cm tissue culture plates in 10 ml of DMEM-10% FCS medium using 10 g of retroviral construct, 4 g of pKAT and 2 g of VSVG plasmid. The virus collection and infection of target cells was carried out essentially as described above for lentiviral vectors.

### Cell Lines and Cells

K562, chronic myelogenous leukemia (CML), Raji (Burkitt's lymphoma), HEL 92.1.7, (erythroleukemia; also called HEL), and RS;411 (acute lymphoblastic leukemia; also called RS411) cell lines were obtained from ATCC and maintained in RPMI1640 medium with 10% FBS. Jurkat (acute T cell leukemia) cell line was obtained from Dr Shao-Cong-Sun laboratory (Pennsylvania State University College of Medicine) and maintained in RPMI1640 medium with 10% FBS. Primary Effusion Lymphoma (PEL) derived cell lines BC-1, BC-3 and BCBL-1, which are infected with Kaposi's sarcoma associated herpes virus, were obtained from Dr. J. Jung (University of Southern California; USC) and maintained in RPMI1640 with 20% FBS. KSHV-transformed primary rat embryonic metanephric mesenchymal precursor (MM; a gift from Dr. S. J. Gao, University of Southern California) were cultured in DMEM 10% FBS. Hodgkin lymphoma cells (L428, L540, L1236, KMH2) were obtained from Dr. Mapara and cultured in RPMI 20% FBS. SK-BR-3 (breast adenocarcinoma) was obtained from ATCC and grown in McCoy's 5a medium with 10% FBS. Other cell lines were obtained from ATCC and maintained in the medium recommended by the supplier. Buffy coat cells were obtained from the Blood Bank at Children Hospital of Los Angeles and used to isolate peripheral blood mononuclear cells (PBMC) by Ficoll-Hypaque gradient centrifugation. PBMC were subsequently used to isolate T cells using CD3 magnetic microbeads (Miltenyi Biotech) and following the manufacturer's instructions. All cells were cultured at 37° C., in a 5% CO2 humidified incubator.

### Antibodies and Drugs

Blinatumomab was obtained from Amgen, Brentuximab vedotin (Adcetris) was obtained from Seattle Genetics, Rituximab was obtained from Genentech. BIIB021 was from Selleck chemicals and JQ1 was provided by Dr. James Bradner (Dana Farber Cancer Institute). Digitonin was purchased from Sigma (Cat. no D141) and a stock solution of 100 mg/ml was made in DMSO. A diluted stock of 1 mg/ml was made in PBS. Unless indicated otherwise, final concentration of Digitonin used for cell lysis was 30 μg/ml.

### Assay for Copepods Luciferases

A 100× stock solution of native coelenterazine (CTZ; Nanolight, cat #303) was made by dissolving 1 mg of lyophilized CTZ powder in 1.1 ml of 100% Methanol supplemented with 30 μl of 6N HCl to avoid oxidation of CTZ with time. To make CTZ assay buffer, the 100× stock solution of CTZ was diluted to 0.5× concentration in PBS. Unless indicated otherwise, a total volume of 15 μl of the CTZ assay buffer (as prepared above) was added using an auto-injector to each well of a 384-well white plate (Greiner, 384 well white plate cat #781075) containing cells expressing the non-secretory form of a copepod luciferase in approximately 50-60 μl volume of medium and plates were read for luminescence in well-mode using BioTek synergyH4 plate reader. In the well-mode, an auto-injector or dispenser is used and assay buffer is added in one well, luminescence is read in that well, then the assay buffer is added to next well and luminescence is read in that well. Where indicated, assay was also conducted in the endpoint mode. In the endpoint mode, an autoinjector or dispenser is not used, the assay buffer is added in all wells manually using a pipette and then luminescence is read in all wells of the plate. For 96 well plates, cells were plated in 200 μl of media and approximately 50 μl of 0.5×CTZ assay buffer was added. Unless indicated otherwise, the 0.5×CTZ assay buffer was used for assaying the activity of copepod luciferase (e.g., GLuc, NLuc, MLuc7, HTLuc, PaLuc1, PaLuc2, MpLuc1, McLuc1, MaLuc1, MoLuc1, MoLuc2, MLuc39, PsLuc1, LoLuc1-3, HtLuc2, TurboLuc16 (TLuc), and Lucia Luc). The CTZ assay buffer (diluted to 0.125× concentration) was also used for measurement of NLuc activity in most experiments. Unless indicated otherwise, the volume of 0.5×CTZ assay buffer added was approximately ¼^{th} of the volume of the liquid in the well containing the cells, although the assay also worked when the 0.5×CTZ assay was added to the media containing the cells in 1:1 volume.

### Nluc Assay

Nano-Glo® Luciferase Assay System (Promega) was used for measurement of NLuc activity in some experiments by following the manufacturer's instructions. Briefly, Nano-Glo® Reagent was prepared by adding 5 μl of Nano-Glo® substrate in 1 ml of Nano-Glo® assay buffer (Promega). 15 μl of Nano-Glo® Reagent prepared above was added directly either manually or using auto-injector to each well of a 384-well white plate containing cells in 60 μl of media. For 96 well plates, cells were plated in 200 μl of media and approximately 50 μl of assay buffer was added. Plates were read for luminescence in endpoint mode using BioTek synergyH4 plate reader without prior cell lysis. In some experiments, NLuc activity was measured using CTZ assay buffer but here the buffer was diluted to final concentration of 0.125×. When CTZ assay buffer was used for measurement of NLuc activity, a total volume of approximately 15 μl (unless indicated otherwise) of the 0.125×CTZ assay buffer was added by auto-injector to each well of a 384-well white plate (Greiner, 384 well white plate cat #781075) containing cells in approximately 50-60 μl volume of medium and plates were read for luminescence using BioTek synergyH4 plate reader. For 96 well plates, cells were generally plated in 200 μl of media and approximately 50 μl of 0.125×CTZ assay buffer was added.

### Measurement of Maximum Cell Death

For measurement of maximum cell death, cells were treated with 301 of Digitonin dissolved in DMSO (final concentration 30 μg/ml) for approximately 90 min prior to measurement of activities of luciferases, such as GLuc, NLuc, TurboLuc, MLuc7, ovaliformis, H. tanneri and renilla luciferases. Percentage Specific lysis was calculated using the luciferase activity of Digitonin-treated cells as maximum cell death and untreated cells as spontaneous cell death and using the formula % specific lysis=100×[(experimental data−spontaneous cell death)/(maximum cell death−spontaneous cell death)].

### Renilla Luc Assay

Unless indicated otherwise, for measurement of Renilla Luciferase activity, the Renilla Luciferase Assay System Kit from Promega (Cat #E2820) was used. Renilla Luciferase Assay Reagent was prepared by adding 1 volume of 100× Renilla Luciferase Assay Substrate to 100 volumes of Renilla Luciferase Assay Buffer and the reagent used in the assay as per manufacturer's instructions. In some experiments, Renilla Luciferase activity was also measured using CTZ assay buffer.

### Assay for Beetle Luciferase (for Fire Fly-Luc (FfLuc or Fluc), LucPPe, LucPpL and CBGR-Luc)

Unless indicated otherwise, for measurement of FFLuc, CBGRLuc and LucPPe-146-1H2 activities, a 10× Luciferin stock solution was prepared consisting of 1 mM D-luciferin synthetic crystalline (Sigma), 25 mM glycylglycine, pH 7.8. A stock solution of luciferin assay buffer was prepared containing 25 mM glycylglycine, pH 7.8, 15 mM potassium phosphate, pH 7.8, 15 mM MgSO₄, 4 mM EGTA, 2 mM ATP (Sigma). Working solution of Luciferase assay buffer for each 1.0 ml consisted of 885.5 μl assay buffer+1 μl DTT (1M stock)+100 μl of 10× Luciferin stock solution+13.5 μl ATP (100 mM stock). The assay buffer containing substrate was generally added to the media containing cells or cellular supernatants at 1:1 (v/v) ratio, unless indicated otherwise.

### Cell Lysis

Unless indicated otherwise, cell lysates were prepared in 1× Renilla Luciferase Assay Lysis Buffer immediately before performing assays by diluting one volume of 5× Renilla Luciferase Assay Lysis Buffer (Promega, Cat #E2820) into four volumes of distilled water. Cell lysis was performed following the manufacturer's instructions.

Transient Transfection of 293FT Cells with Retro- and Lentiviral Based Luciferase Expression Vectors

The 293FT cells were transfected in a 24-well plate with various expression plasmids (500 ng/well) using calcium phosphate. Approximately, 18 hours post-transfection, the cells were either left untreated or treated with 30 μg/ml Digitonin to induce cell death, followed by the detection of luminescence as described under luciferase assays.

### Generation of NK92MI Cells Stably Expressing CARs

NK92MI cells stably expressing second generation CAR constructs and coexpressing EGFP were generated by infecting with concentrated lentiviruses as described for primary human T cells. The EGFP positive cells were sorted using BD FACSAria II and expanded.

### Example 2

### Construction and Testing of Vectors Expressing Gaussia Luciferase (GLuc) and NanoLuc (NLuc) for Use in Matador Cytotoxicity Assay

We cloned the a gene fragment encoding GLuc lacking its signal peptide (amino acid residues 1-17) and encoding its amino acid residues 18 to 185 preceded by an ATG start codon in retroviral vectors MSCVneo and MSCVhygro and lentiviral vector pLenti-Blast. We also expressed the GLuc cDNA lacking the signal peptide from a lentiviral vector pLenti-EF1α in which its expression was driven by the constitutively active Elongation Factor 1-alpha promoter. This construct also expressed the puromycin resistance gene (pac) upstream and in-frame with the GLuc cDNA and separated from it by a T2A ribosomal skip sequence. The nucleotide sequences of GLuc constructs pLenti-EF1α-Pac-T2A-Gluc-B07, MSCVhygro-GLuc-HA-G02, MSCVpac-GLUC-HA-R03 and pLENTI-Gluc-Flag-blast-B07 are provided in SEQ ID NOs: 94, 95, 96 and 98, respectively. We generated polyclonal population of several cell lines stably expressing GLuc and confirmed GLuc expression by measuring the GLuc luciferase activity in the cell lysates following addition of an assay buffer containing coelentrazine. In general the level of expression of GLuc was higher in cells infected with the lentiviral vectors as compared to those infected with the retroviral (i.e. MSCV) vectors. We also cloned the NanoLuc (NLuc; Promega) cDNA lacking a signal peptide into the pLenti-Blast vector, in which NLuc expression was being driven by CMV promoter, and used this vector to generate cells stably expressing NLuc. The DNA sequence of this vector is provided in SEQ ID NO: 92.

### Example 3

Gluc can be Used in Matador Cytotoxicity Assay to Measure Cell Death with Sensitivity Down to a Single Cell Level

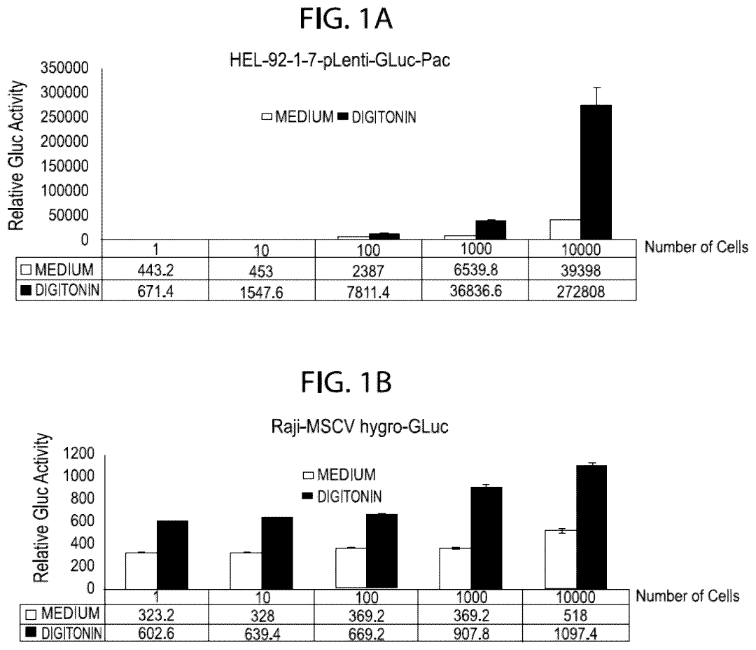

HEL-92-1-17 (obtained from ATCC) cells stably expressing GLuc from the pLenti-EF1α-PAC-T2A-GLuc-B07 vector (SEQ ID NO: 94) were grown in complete medium (RPMI 1640 media+10% FBS). Cells were washed with growth medium, and diluted to 3.3×10⁶ cells/ml in complete medium (100K cells/30 l). Cells were then serially diluted tenfold in complete medium to a final concentration of 10,000 cells/30 μl, 1000 cells/30 μl, 100 cells/30 μl and 1 cell/30 μl. A total volume of 30 μl of cells was plated per well in 10 wells of white 384-well plates for each concentration. The cells were either treated with 30 μl of PBS (n=5 wells at each cell concentration) or treated (n=5 wells at each cell concentration) with 30 μl of Digitonin (final concentration 30 μg/ml) and incubated for 1 hour at 37° C. in a 5% CO₂ incubator. A total volume of 15 μl of 0.5×CTZ assay buffer was added by autoinjector directly to each well of the 384-well white plate containing cells in 60 μl of volume (30 μl of RPMI-10% FBS+30 μl of Digitonin) without prior cell lysis and plates were read for luminescence using BioTek synergy H4 plate reader in well mode. FIG. 1 shows that induction of cell death by treatment of HEL-92-1-17 cells stably expressing GLuc with Digitonin resulted in an increase in GLuc luciferase activity, which was evident even in wells containing a single cell. Essentially similar results were obtained upon Digitonin treatment of RAJI cells stably expressing GLuc from the MSCVhygro-GLuc-HA-G02 vector (SEQ ID NO: 95). Thus, GLuc is not degraded by proteases released during cell death, making it a suitable candidate for the development of an assay of cellular cytotoxicity. These results further demonstrate that the CTZ assay buffer (CTZ dissolved in PBS) is not cytotoxic to cells as otherwise the GLuc activity in the samples with and without Digitonin treatment would have been similar, making it impractical to conduct the assay in a homogenous format.

### Example 4

Nluc can be Used in Matador Cytotoxicity Assay to Measure Cell Death with Sensitivity Down to a Single Cell Level

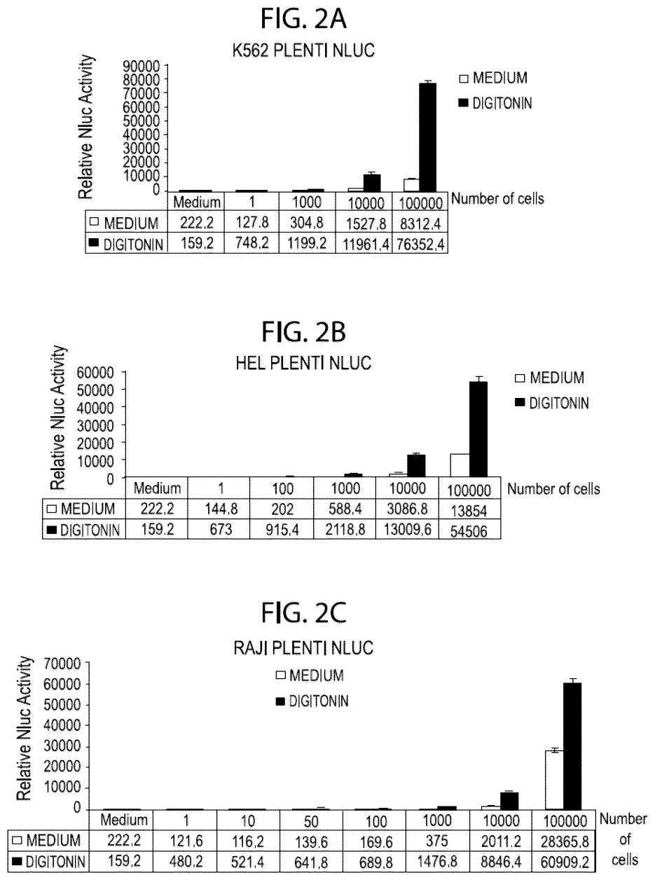

A similar experiment was conducted with K562, HEL-91-1-17 and RAJI cells stably expressing NLuc from pLENTI-NLuc-AcV5-Blasticidin-Pa08 vector (SEQ ID NO: 92). Cells plated in 30 μl of medium per well were either treated with 30 μl of PBS or 30 μl of PBS containing Digitonin (final concentration 30 μg/ml) for 1 hour at 37° C., in a 5% CO₂ incubator. For measurement of NLuc activity, 0.5×CTZ assay buffer was added by automated injector directly to each well of the 384-well white plate containing cells in 60 μl of volume (30 μl of RPMI-10% FBS+30 μl of Digitonin) without prior cell lysis and plates were read for luminescence using BioTek synergy H4 plate reader in well mode. FIG. 2 shows that Digitonin treatment of NLuc expressing K562, HEL-91-1-17 and RAJI cells resulted in increase in NLuc activity, which was evident even in wells containing a single cell. Furthermore, even though there was a progressive increase in the NLuc activity in both the untreated and Digitonin-treated samples with increase in cell number, the ratio (fold increase) of NLuc activity between Digitonin-treated and untreated (medium alone) samples was relatively stable and independent of the number of plated cells. Thus, NLuc is not degraded by proteases released during cell death, making it a suitable candidate for the development of an assay of cellular cytotoxicity.

### Example 5

### Use of MLuc7 in Matador Cytotoxicity Assay

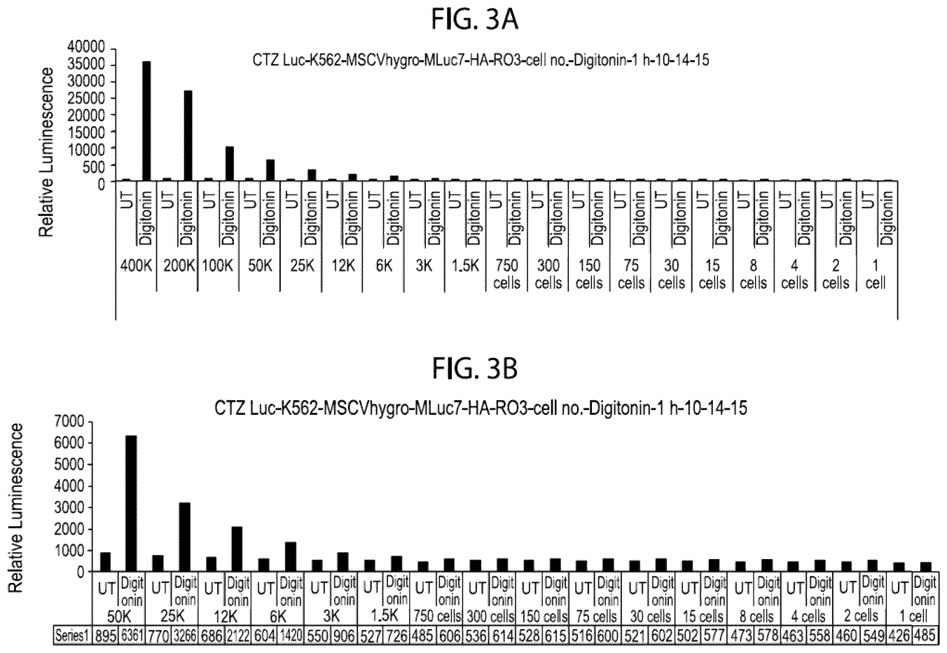

To test if other copepod luciferases may be used similarly to GLuc for development of Matador cellular cytotoxicity assay, we cloned a gene fragmentencoding the non-secretory (lacking the signal peptide) form of Metridia longa luciferase 7 (MLuc7) into MSCVhygro vector. The MLuc7 gene fragment (SEQ ID NO: 4) also carried M43L and M110L substitutions. The corresponding substitutions in GLuc have been previously shown to result in Glow type luminescence suitable for high throughput applications (Welsh, Patel et al. 2009). Stable population of K562 cells expressing MLuc7-M43L-M110L mutant and carrying a carboxy terminal HA epitope tag were generated by retroviral mediated gene transfer and selection with hygromycin. K562-MLuc7-expressing target cells were washed with growth medium, and diluted to 4.0×10⁶ cells/ml in complete medium. Cells were then serially diluted in complete medium to a final concentration of 200K cells/30 μl, 100K cells/30 μl, 50K cells/30 μl, 25K cells/30 μl, 12K cells/30 μl, 6K cells/30 μl, 3.0K cells/30 μl, 1.5K cells/30 μl, 750 cells/30 μl, 300 cells/30 μl, 150 cells/30 μl, 75 cells/30 μl, 30 cells/30 μl, 15 cells/30 μl, 8 cells/30 μl, 4 cells/30 μl, 2 cells/30 μl and 1 cells/30 μl. A total volume of 30 μl of target cells was plated per well in 3 wells for each concentration in a Greiner 384-well flat bottom white opaque plate. The cells were either treated with Digitonin for measurement of maximum cell death or left untreated and incubated for 1 hour in a 37° C., 5% C02 incubator. The luciferase activity in K562-MLuc7-expressing cells was precisely measured by BioTek synergy plate reader that directly injected 15 μl of the 0.5×CTZ assay buffer into the 384 well plates in well mode. FIG. 3 shows that induction of cell death by Digitonin results in increase in MLuc7 activity that was detectable even at 1 cell/well level and there was a progressive increase in MLuc7 activity with increase in cell number up to 400K cells/well. The lower panel is derived from the data in the upper panel and focuses on the range from 1 cell/well to 50K cells/well. Even though the MLuc7-M43L-M110L mutant was used in the above example, we believe that the non-secretory form of wild type MLuc7 could be used in the development of the above assay as well. Thus, MLuc7 is not degraded by proteases released during cell death, making it a suitable candidate for the development of the Matador assay of cellular cytotoxicity.

### MLuc7 Assay (Additional Example)

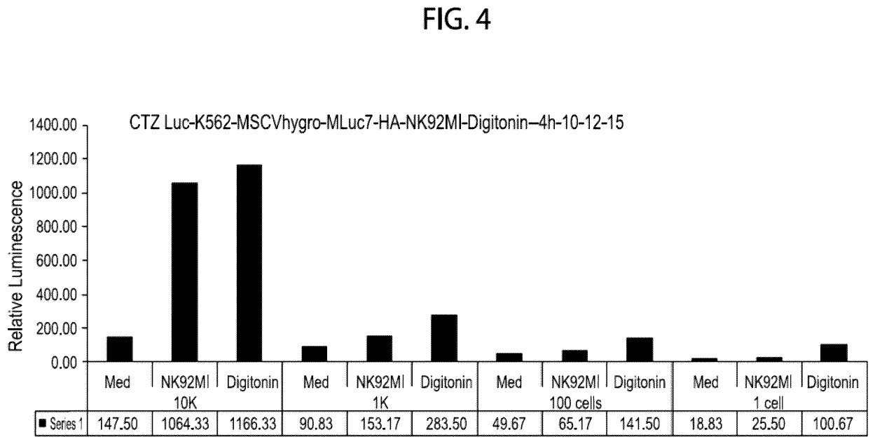

K562-M7-Luc (MSCVhygro-MLuc7-MM-LL-HA-R03)-expressing target cells were plated at a final concentration of 10K cells/30 μl, 1K cells/30 μl, 100 cells/30 μl, and 1 cell/30 μl. A total volume of 30 μl of target cells was plated per well into 3 wells for each concentration in a Greiner 384-well flat bottom white opaque plate. The cells were either left untreated or co-cultured with NK92MI effector cells at a Effector (E) to Target (T) ratio of 0.5:1 and incubated for 4 hours in a 37° C., 5% CO2 incubator. Cells were treated with Digitonin (final concentration 30 μg/ml) for maximum cell death. Total volume in each well was 60 μl where Effector and Target cells were co-cultured and 30 μl of culture media was added in other wells to make a total volume of 60 μl in all the wells. The luciferase activity in K562-MLuc7-expressing cells was precisely measured by BioTek synergy plate reader by directly injecting 15 μl 0.5×CTZ assay buffer containing native coeloentrazine (Nanaolight) into the 384 well plates in a well mode. FIG. 4 shows that induction of cell death by Digitonin and NK92MI cells results in increase in MLuc7 activity that was detectable even at 1 cell/well level and there was a progressive increase in MLuc7 activity with increase in cell number up to 10K cells/well. These results further demonstrate that MLuc7 activity is stable even under conditions when cell death is induced by NK92MI cells. Thus, cell death proteases released during NK92MI-induced cell death do not degrade MLuc7 and this luciferase can be used for the development of an assay of cellular cytotoxicity.

### Example 6

### Use of TurboLuc16 (TLuc) in Matador Cytotoxicity Assay

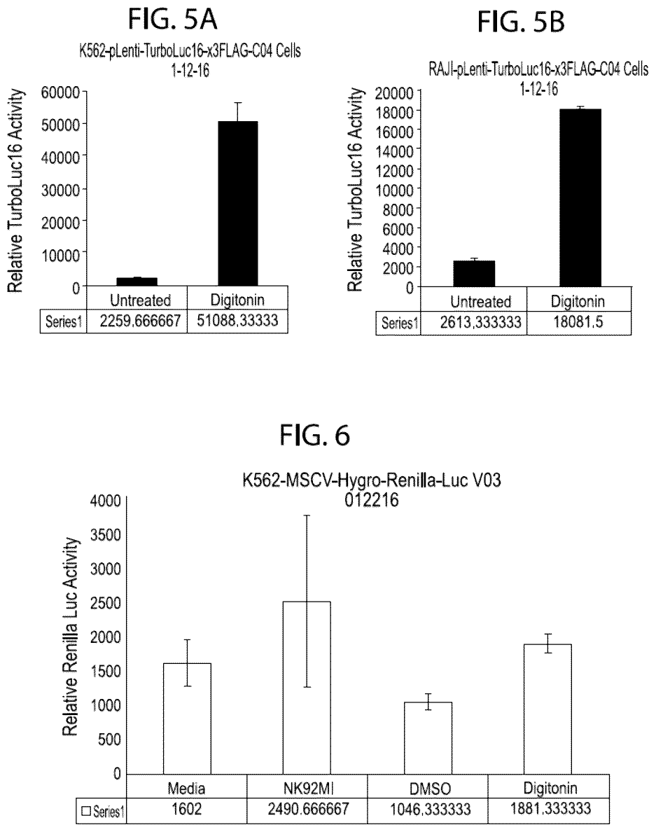

Stable population of K562 and RAJI cells expressing TurboLuc16 (TLuc) and carrying a carboxy terminal ×3FLAG epitope tag were generated by lentiviral mediated gene transfer of pLENTI-TurboLuc-16-X3Flag-Blast-C04 construct (SEQ ID NO: 93) and selection with blasticidin. Cells were plated in 384 well plates (25,000 cells/well) in 25 μl media and treated with 5 μl of PBS or PBS containing Digitonin (5 μl) for 90 min. The TurboLuc activity was measured by BioTek synergy plate reader by directly injecting 30 μl of 0.5×CTZ assay buffer containing native coeloentrazine into the 384 well plates in well mode. FIG. 5 shows that induction of cell death by Digitonin results in increase in TLuc activity in both K562 and RAJI cells. Thus, TLuc is not degraded by proteases released during cell death and it can be used for the development of an assay of cellular cytotoxicity.

### Example 7

### Use of Renilla Luc in Matador Cytotoxicity Assay

K562 cells were infected with MSCVhygro-Renilla-Luc retroviral vector and selected in hygromycin to generate polyclonal population of cells stably expressing Renilla Luc in the cytosol. Approximately 2.5×10⁴ cells were plated in each well of a 384 well plate in 20 μl of medium. Cells were either treated with 20 μl of medium alone, 20 μl of medium containing NK92MI cells (E:T ratio of 1:1), 20 μl of DMSO (control) or 20 μl of Digitonin dissolved in DMSO. Treatment with Digitonin was for 2 hours, while all other treatments were for approximately 4 hours. Cell death was measured by injection of 0.5×CTZ assay buffer. FIG. 6 shows increase in Renilla Luc activity upon induction of cell death by NK92MI cells or by treatment with Digitonin.

### Example 8

### Use of CBGRluc in Matador Cytotoxicity Assay

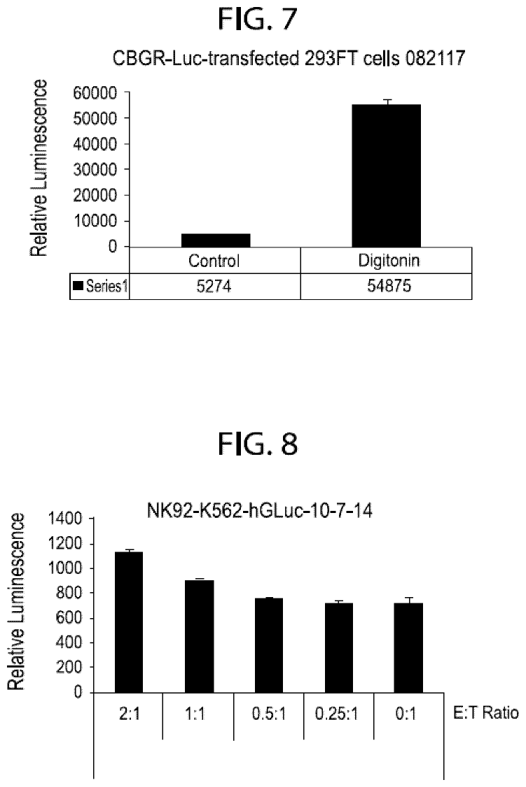

293FT cells were transfected with pLenti-CBGRluc construct in a 100 mM plate. 72 hours post-transfection, cells were trypsinized and plated in 6-well plates. For induction of cell death, cells in some wells were treated with Digitonin (50 μg/mL final concentration). CBGRluc activity was determined by adding assay buffer (1 mM D-Luciferin, 25 mM glycylclycine, 15 mM potassium phosphate, 15 mM MgSO₄, 4 mM EGTA, 2 mM ATP, and 1 mM DTT) in a well mode. The assay buffer with substrate was added at 1:1 (v/v) ratio. FIG. 7 shows increase in CBGRluc activity upon induction of cell death with Digitonin. Thus, CBGRLuc is not degraded by proteases released during cell death and it can be used for the development of an assay of cellular cytotoxicity.

### Example 9

### Use of Matador Assay to Measure Cell-Induced Cytotoxicity Mediated by NK Cells

To demonstrate that Matador assay can be used to measure cell induced cytotoxicity, K562-GLuc cells were used as a target for cytotoxicity mediated by the natural killer derived cell line NK92MI (effector cells). K562-GLuc cells were plated at a concentration of 100,000 cells/well and co-cultured for 24 h with NK92MI cells at E:T ratios of 2:1, 1:1, 0.5:1, 0.25:1 and 0:1, respectively. At the end of the culture period, GLuc activity was measured by addition of coelentrazine containing assay buffer to the wells in well mode. FIG. 8 shows that there is a progressive increase in GLuc activity with increase in E:T ratio of NK92MI cells.

### Example 10

### Matador Assay can be Used to Measure Cytotoxicity in a Single Step Manner (Homogenous)

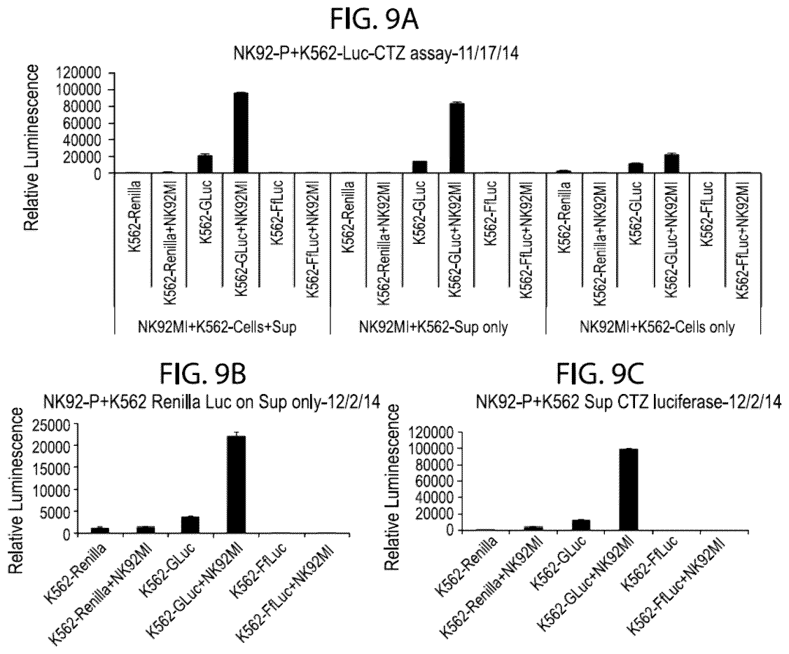

The NK92-sensitive K562 cells were engineered to express either a Gaussia princeps (GLuc) or a Fire-fly (FFLuc), luciferase construct by transducing these cells with MSCVhygro-GLuc or pRetro-FFLuc encoding viruses. Cells were selected with hygromycin and puromycin, respectively, to make stable populations of K562-GLuc and K562-FFLuc target cells, respectively. K562 were also co-transfected with phRGTK Renilla Luciferase (Promega) with pCMV-puro plasmid DNA and selected with puromycin to generate K562-Renilla Luc cells. K562-GLuc, K562-FFLuc and K562-Renilla Luc cells were plated either alone or in combination with NK92MI effector cells at concentration of 100K cells/well in 2 wells of a flat-bottom 24 well plate in 500 μl of phenol-red-free RPMI medium at a E:T ratio of 0.5:1. After 4 hours of co-culture, the cells were mixed well, collected in 1.5 ml microfuge tubes and divided into 3 groups of 166 μl each. From the first group, the cells with cell supernatant were directly assayed for luciferase activity by plating 50 μl of cells/well in a white 384 well plate in triplicate. Second group of 166 μl of cells were centrifuged and only cell supernatants were collected in a new tube and plated at 50 μl/well in a 384 well plate in triplicate. The third group samples were centrifuged, supernatant was removed, cells were re-suspended in PBS to assess luciferase activity in the cells by plating 50 μl of re-suspended cells per well in 384-well plate in triplicate. The luciferase activity was precisely measured by BioTek synergy plate reader that directly injecting 0.5×CTZ assay buffer containing native coelentrazine (Nanaolight) into the 384 well plates in a well mode. FIG. 9A shows that induction of K562-GLuc cell death by NK92MI cells results in significant and near equivalent increase in GLuc activity when measured either by directly adding 0.5×CTZ assay buffer to the wells containing cells plus supernatant or when the assay buffer is added to supernatant that had been cleared of cells by centrifugation step. These results demonstrate that the centrifugation step to collect the supernatant freed of cells may be omitted without significantly compromising the sensitivity of the Matador assay, thereby yielding a single step homogenous assay. There was also a slight increase in GLuc activity in the pellet of K562-GLuc cells that had been incubated with NK92MI cells. This could be due to the fact that loss of cell membrane integrity results in increased penetration of coelentrazine into the dead and dying cells where it interacts with any GLuc that has been trapped inside the cells and has not yet leaked out.

FIG. 9A also shows that induction of death of K562-Renilla Luc cell by NK92MI cells also resulted in a slight increase in Renilla luciferase activity when measured either by directly adding 0.5×CTZ assay buffer to the wells containing cells plus supernatant or when added to supernatant that had been cleared of cells by the centrifugation step.

FIG. 9A also shows that addition of 0.5×CTZ assay buffer to cells plus supernatant fraction, supernatant only fraction or cell pellet only fraction of K562-FFluc cells did not result in an increase in luciferase activity, which is consistent with the fact that coelentrazine is not a substrate for firefly luciferase.

To test whether the GLuc and Renilla Luc activities are stable after freezing and thawing, the supernatant fractions collected from K562-GLuc, K562-Renilla-Luc and K562-FFluc cells that had been assayed above were frozen. Two weeks later, the supernatants were thawed and then re-assayed after addition of coeloentrazine. As shown in FIG. 9B, there was no major change in the GLuc and Renilla Luc activities as compared to the results obtained in FIG. 9A.

The supernatant fractions after thawing was also assayed using the Renilla Luciferin substrate (Promega). FIG. 9C shows that the assay conducted using the Renilla Luciferin substrate gave similar results as those obtained after addition of 0.5×CTZ assay buffer containing native coeloentrazine.

### Example 11

### Matador Assay can be Used as a Single Step Homogeneous Assay

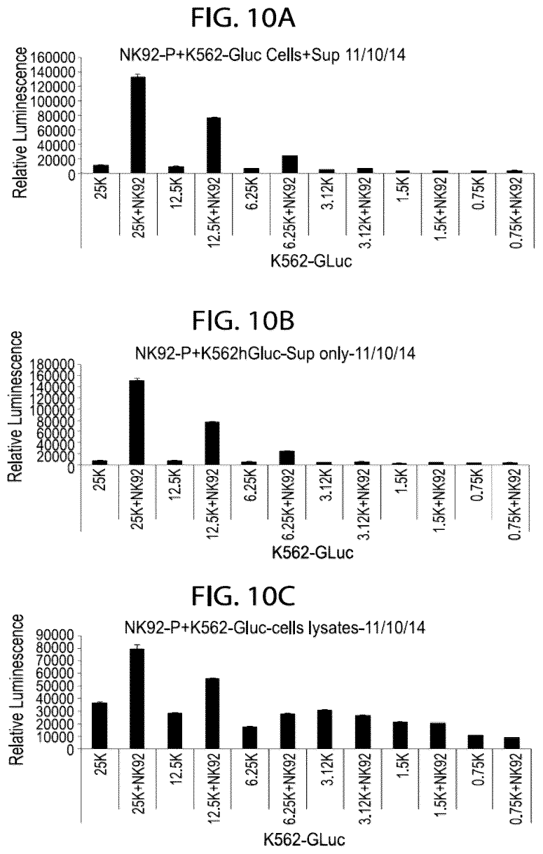

K562-GLuc cells were plated either alone or in combination with NK92MI effector cells at the indicated cell concentrations/well in 2 wells of a flat-bottom 24 well plate in 500 μl phenol-red-free RPMI medium. The effector to target cell ratio (E:T) was kept constant at 0.5:1 in each well. After 4 hours of co-culture, the cells were mixed well, collected in 1.5 ml microfuge tubes and divided into 3 groups of 166 μl each. From the first group, the cells with cell supernatant were directly assayed for luciferase activity by plating 50 μl/well in a white 384 well plate in triplicate. Second group of 166 μl cells were centrifuged and only cell supernatants were collected in new tubes and plated in a 384 well-plate at 50 μl/well in triplicate. The third group of samples were centrifuged, supernatants were removed, cells were re-suspended in PBS to assess luciferase activity by plating 50 μl of re-suspended cells per well in a 384-well plate in triplicate. For fractions derived from K562-GLuc (FIGS. 10A-10C), the GLuc activity was precisely measured by BioTek synergy plate reader by directly injecting 15 μl of 0.5×CTZ assay buffer containing native coeloentrazine into the 384 well plates in well mode. FIG. 10A-10B show that there is a progressive increase in GLuc activity in both the supernatant fraction and the cells plus supernatant fraction with increase in number of target cells from 0.75K to 25K. Additionally, there was a near equivalent increase in GLuc activity when measured either by directly adding 0.5×CTZ assay buffer to the wells containing cells plus supernatant or when added to the supernatant fractions that had been cleared of cells by centrifugation step. These results confirm that the centrifugation step to collect the supernatant freed of cells can be omitted without compromising the sensitivity of the assay, thereby yielding a single step homogenous assay. Finally, there was also an increase in GLuc activity in the pellet of K562-GLuc cells that have been incubated with NK92MI cells (FIG. 10C; compare lanes showing result with 6.25K, 12.5K and 25K cells with and without NK92MI cells). This could be due to the fact that loss of cell membrane integrity results in increased penetration of coeloentrazine into the dead and dying cells where it reacts with any GLuc that had been trapped inside the cells and has not yet leaked out. Thus, the cytotoxicity assay described in this invention when performed in the homogeneous format is based not only on the release of the reporter (e.g., GLuc or NLuc) into the extracellular compartment but also on the increased penetration of the substrate required for the reporter activity into cells that have lost membrane integrity.

### Example 12

### Use of Matador Cytotoxicity to Monitor Cytotoxicity Induced by Chimeric Antigen Receptor Modified NK Cells

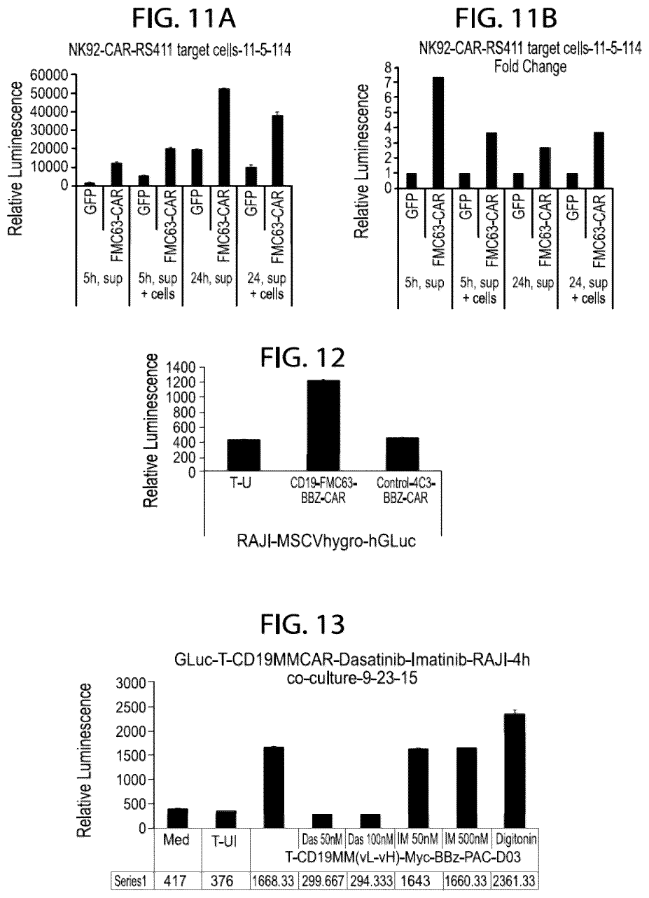

RS411-GLuc cells were plated at concentration of 1×10⁶ cells/well either alone or in combination with NK92MI effector cells expressing either an empty vector (GFP) or a chimeric antigen receptor targeting CD19 (FMC63-MYC-BBZ-T2A-EGFP; SEQ ID NO: 79)) in 2 wells of a flat-bottom 24 well plate in 500 μl phenol-red-free RPMI medium. The effector to target cell ratio (E:T) was kept constant at 0.25:1 in each well. After the indicated time intervals, fractions containing cells plus supernatant, supernatant only or cells only were collected and 50 μl of each fraction was plated in 384 well plate as described in the preceding sections. GLuc activity was measured by injecting 15 μl of 0.5×CTZ assay buffer in a well mode. FIG. 11A shows the absolute values while the FIG. 11B shows fold increase in activity when normalized relative to the activity observed in RS411-GLuc cells that had been incubated with NK92MI cells expressing GFP. The results show that Matador cytotoxicity assay can be used to monitor cytotoxicity induced by CAR-expressing effector cells and the assay can be performed as a single step homogenous assay (i.e. cells+supernatant) format by directly adding 0.5×CTZ assay buffer to the wells or by adding 0.5×CTZ assay buffer to the supernatant fraction that has been separated from the cells.

### Example 13

Use of Matador cytotoxicity to monitor cytotoxicity induced by chimeric antigen receptor modified T (CAR-T) cells

CD19-expressing Raji-MSCV-hygro-GLuc cells were cultured for 4 hours with uninfected T cells (T-UI) or T cells that had been infected with lentiviral vectors expressing chimeric antigen receptors FMC63-BBZ (directed against human CD19 antigen) and 4C3-BBZ (negative control CAR targeted against an irrelevant protein). GLuc activity was measured by addition of coelentrazine assay buffer to the wells as described above. The results (FIG. 12) show that induction of RAJI cell death by T cells expressing CD19-specific CAR-T results in increase in GLuc activity, whereas no increase in GLuc activity was observed by co-culture with uninfected T cells or those expressing a negative control CAR (i.e. 4C3) whose target antigen is not expressed in RAJI cells. Essentially similar results were obtained by T cells expressing multiple additional CARs (e.g. targeting CD20, CD22, CD123, FR1 etc) when co-cultured with their target cells expressing different marine luciferases (e.g. GLuc, NLuc etc.).

### Example 14

### Use of Matador Assay for Measuring Cytotoxicity by Chimeric Antigen Expressing Cells and its Inhibition by Drug Treatment

Human peripheral blood T cells isolated using CD3 magnetic beads were infected with lentiviruses expressing the CAR construct CD8SP2-CD19MM-(vL-vH)-Myc-BBz-T2A-PAC [SEQ ID NO: 82] targeting CD19 and selected with puromycin. CAR-T cells were pre-incubated with the indicated concentrations of Dasatinib and Imatinib for approximately 30 min at 37° C. The drug-treated and untreated T cells were plated in a white 384-well plate. RAJI cells stably expressing GLuc were added to the wells containing the T cells at a concentration of 30K cells/well to give an E:T ratio was 5:1. Dasatinib (Das) and Imatinib (Imat) were added to the wells to maintain the final concentrations as indicated. After 4 hours of co-culture, CAR-T cells mediated induction of lysis of target cells was assayed by increase of GLuc activity by directly injecting 0.5×CTZ assay buffer containing native coeloentrazine in a well mode. FIG. 13 shows inhibition of CAR CD8SP2-CD19MM-(vL-vH)-Myc-BBz-T2A-PAC-induced cell death by 50 nM and 100 nM Dasatinib, while Imatinib has no effect. These results demonstrate that Matador cytotoxicity assay can be used to study cytotoxicity by CAR-T cells and its modulation by drug treatment. Therefore, the assay can be used to screen for and identify candidate compounds that modulate the activity of cytotoxic cells, such as cytotoxic T cells, NK cells, CAR-T cells and T cells expressing engineered TCRs.

### Example 15

### Use of Matador Cytotoxicity Assay for Measuring Death of Solid Tumor Derived Cells

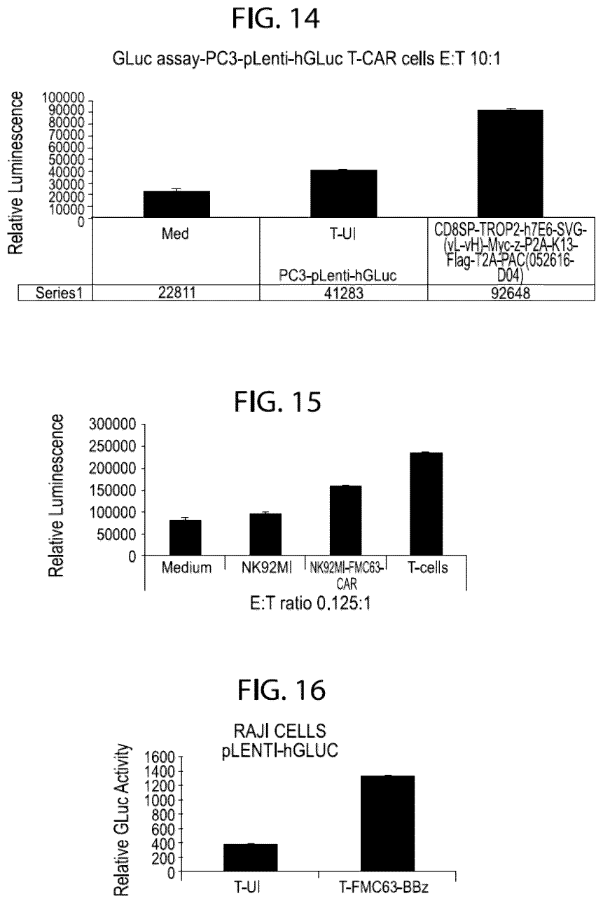

Prostate cancer derived PC3 cells were grown as adherent culture in DMEM, 10% FCS. Human peripheral blood T cells isolated using CD3 magnetic beads were infected with lentiviruses expressing the construct CD8SP-TROP2-h7E6-SVG-(vL-vH)-Myc-z-P2A-K13-Flag-T2A-PAC [SEQ ID NO: 84] targeting TROP2 and selected with puromycin and tested for the ability to kill PC3 (prostate cancer) cells expressing GLuc using the assay described previously. PC3-Gluc cells were plated in a flat bottom 384 well plate as adherent culture and co-cultured overnight with the CAR-T cells targeting TROP2 at an E:T ratio of 10:1. CAR-T cells mediated induction of lysis of target cells was assayed by increase of GLuc activity by directly injecting 0.5×CTZ assay buffer containing native coeloentrazine using automatic injector. FIG. 14 shows effective killing of PC3 cells by T cells expressing the CD8SP-TROP2-h7E6-SVG-(vL-vH)-Myc-z-P2A-K13-Flag-T2A-PAC CAR construct as measured by increase in GLuc activity, thereby demonstrating the utility of the Matador assay to monitor death of solid-tumor derived cells grown as adherent culture.

### Example 16

### Use of Matador Assay for Measuring Cellular Cytotoxicity Against Primary Cells

The preceding examples showed that the Matador assay can be used to measure death of established cell lines. To examine if this assay can be also used to measure death of primary cells, leukemic blasts were isolated from a de-identified leukemia patient using Ficoll-Hypaque gradient centrifugation and then transduced with pLenti-EF1α-Pac-T2A-Gluc-B07 (SEQ ID NO: 94) lentiviral vector to generate primary leukemia blast cells (Chrt3) expressing the non-secretory form of GLuc cDNA. Briefly, 1 million primary leukemic blasts were cultured in XVIVO medium in a 6-well plate in the presence of GLuc lentiviral supernatant and polybrene (5 μg/ml) and centrifuged at 2,800 rpm at 32° C. for 2 hours followed by incubation of cells at 37° C. for 24 hours in XVIVO medium. To measure the killing of Chrt3-GLuc cells, the transduced cells were plated at a density of 20,000 cells per well in 30 μl of medium in triplicate in a white 384 well plate and co-cultured with the effector cells for 4 hours at an E:T ratio of 0.125:1. Cell death was assayed by the single-step homogenous GLuc assay by injecting 0.5×CTZ assay buffer in well mode. As shown in FIG. 15, co-culture with NK92MI effector cells expressing the FMC63-BBZ-CAR targeting the CD19 antigen resulted in significant increase in GLuc activity, indicating cytotoxicity. Similarly, co-culture with primary T cells isolated from a healthy unrelated donor resulted in a marked increase in GLuc activity. These results demonstrate that primary patient-derived cells can be effectively modified to express the non-secretory form of GLuc. These results further demonstrate that the GLuc release assay can be used to measure cell death of primary patient derived cells. Finally, primary cells in the above example were used without prior drug selection to select for lentiviral transduced GLuc-expressing cells. Thus, the GLuc release assay can be used on target cells in which only a fraction of cells express the non-secretory form of GLuc and the assay does not require generation of target cells uniformly expressing the non-secretory form of GLuc. Two additional primary leukemia samples are infected with GLuc encoding lentiviral vector and cell death is induced by treatment with Digitonin. Induction of cell death in both cases by treatment with Digitonin is shown to results in an increase in GLuc activity as compared to untreated cells.

### Example 17

Matador Assay can be Used to Measure Death of Target Cells in which Non-Secretory Forms of GLuc is Expressed in Target Cells by Transient Transfection

To determine if transient transfection of a cDNA encoding GLuc can be used to develop Matador cytotoxicity assay, RAJI (CD19+ve) cells were transfected with pLenti-EF1α-Pac-T2A-Gluc-B07 (SEQ ID NO: 94) plasmid DNA using Fugene HD (Promega). Briefly, a transfection mixture was prepared using 97 μl of serum-free RPMI medium with 3 μl of Fugene HD and 2 μg/ml of plasmid DNA, followed by 15 min incubation at room temperature. RAJI cells were plated at a density of 1×10⁶ cells/well in a 24-well plate in 500 μl of serum-free RPMI medium. The cells were transfected by adding the transfection mixture on top of the cells as per manufacturer's instructions and incubated for 48 hours to allow expression of the transfected GLuc cDNA lacking the signal peptide. At the end of 48 hours, RAJI cells transiently expressing GLuc were plated at a density of 100K cells/well in 30 μl medium in a white 384-well plate either alone or in the presence of the indicated effector cells (T cells uninfected, or T cells infected with a lentiviral encoding the FMC63-BBz CAR directed against CD19). After 4 hours, the release of GLuc from dead and dying cells was measured by a luminometer that directly injected native coeloentrazine into the 384 well plates in well mode. As shown in FIG. 16, co-culture of RAJI cells transiently transfected with the non-secretory form of GLuc cDNA with T cells expressing FMC63-BBz chimeric antigen receptor resulted in increase in GLuc activity as compared to uninfected T cells, indicative of cell death.

### Example 18

### Use of Matador Assay to Measure Cell Mediated Cytotoxicity by CAR-Modified NK and T Cells

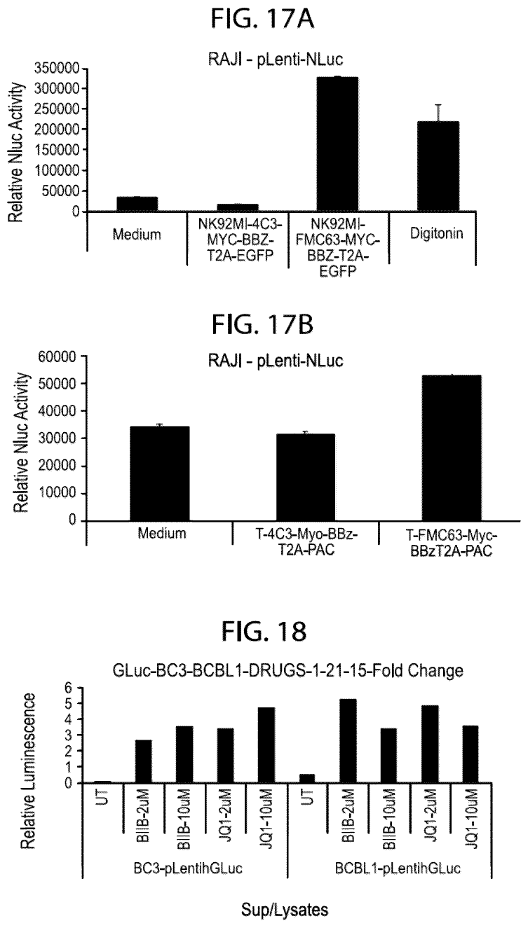

The pLENTI-NLuc-AcV5-Blast virus was used to infect RAJI cells followed by selection with blasticidin to generate stably transduced cells. RAJI-NLuc cells were plated in a 384 well plate at 30K/well in 30 μl of medium. NK92MI cells were infected with a lentiviral CAR construct targeting CD19 (FMC63-Myc-BBz-T2A-EGFP; SEQ ID NO: 79) or a control CAR (4C3-Myc-BBz-T2A-EGFP) and CAR-expressing cells sorted based on EGFP expression. Primary human T cells were infected with a lentiviral CAR construct targeting CD19 (FMC63-Myc-BBz-T2A-PAC; SEQ ID NO: 80) and CAR-expressing cells selected by puromycin selection. Where indicated, parental and CAR-expressing NK92MI effector cells were added at 15K/well in 30 μl of media to give an E:T ratio 0.5:1. Where indicated, parental and FMC63 CAR-expressing T cells were added at 300K/well in 30 μl to give an E:T ratio of 10:1. After approximately 4 hours of co-culture, NLuc activity was measured using BioTek synergy plate reader after injection of 15 μl of 0.125×CTZ assay buffer in well mode. As shown in FIG. 17A, co-culture of RAJI-NLuc cells with NK92MI cells expressing FMC63-BBZ chimeric antigen receptor resulted in significant increase in NLuc activity as compared to co-culture with NK92MI cells expressing a control (4C3-BBZ) chimeric antigen receptor or culture without NK92MI cells (medium alone). FIG. 17B shows that RAJI cells expressing NLuc also showed a significant increase in NLuc activity upon co-culture with T cells expressing FMC63-BBZ chimeric antigen receptor as compared to those expressing the control (4C3-BBZ) chimeric antigen receptor.

### Example 19

### Use of Matador Assay for Measuring Drug Induced Cytotoxicity

To test whether the Matador assay can be used to measure drug induced cell death, BC-3-pLenti-GLuc and BCBL-1-pLenti-GLuc cells that were stably transduced with a lentiviral vector expressing GLuc were treated with different doses of BIIB021 (Hsp90 inhibitor; Selleck chemicals) and JQ1 (BRD4 inhibitor) for 96 hours. Since the duration of drug treatment was relatively long in this experiment, the untreated cells would have continued to proliferate as compared to the drug treated cells. As a result, at the time of GLuc assay, the number of GLuc-expressing cells in the wells that were left untreated was significantly higher as compared to the wells that were drug-treated. To control for the varying number of GLuc-expressing cells in different wells, which could have confounded the results of GLuc cytotoxicity assay due to varying level of spontaneous GLuc release, cells were centrifuged at the end of drug treatments and cellular supernatants were separated from cell pellets. The cell pellets were lysed in 1× Renilla Luciferase Assay Lysis Buffer by diluting one volume of 5× Renilla Luciferase Assay Lysis Buffer (Promega, Cat #E2820) into four volumes of distilled water. GLuc activity was measured in the cellular supernatants and cell lysates fractions separately in triplicate by addition of 0.25× coelentrazine assay buffer (15 μl of assay buffer added to 15 μl of sample in a 384 well plate) to each fraction. For each sample, the average GLuc activity in the cellular supernatant was divided by the average GLuc activity in the cell lysates to normalize for difference in the number of GLuc expressing cells. FIG. 18 shows that treatment of BC-3-pLenti-GLuc and BCBL-1-pLenti-GLuc cells with BIIB021 and JQ1 results in a significant increase in GLuc activity in the supernatant when normalized for the difference in the GLuc-expressing cells as measured by GLuc activity in the cell lysates. As the BC-3 and BCBL-1 cell lines are infected with Kaposi's sarcoma associated herpes virus, the above results demonstrate the utility of the Matador assay to detect cytotoxicity of virally infected cells.

### Example 20

### Use of Matador Assay for Measuring Cellular Cytotoxicity Induced by an Antibody

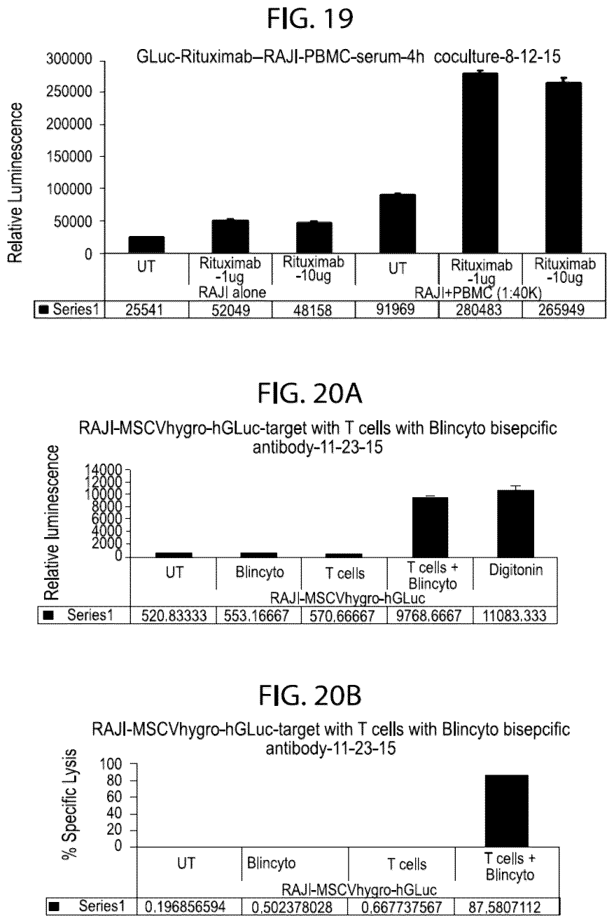

Antibody-dependent cellular cytotoxicity (ADCC) assay was performed essentially as described earlier (Teeling, French et al. 2004). To determine the cytotoxicity of Rituximab monoclonal antibody, the cell death was assessed using Matador assay. Peripheral Blood Mononuclear Cells (PBMC) were isolated by Ficoll-Hypaque gradient centrifugation from buffy coat cells obtained from a de-identified healthy donor as a source of natural effector. Briefly, 1×10⁵ RAJI cells expressing GLuc were plated either alone in a 30 μl volume per well in a white 384 well plates or plated in the presence of 4×10⁶ PBMC. The cells were either left untreated, or treated with 1 μg/ml or 10 μg/ml of Rituximab. The plates were incubated at 37° C. for 4 hours in a CO₂ incubator. After incubation for 4 hours, the cell death was assessed by luciferase assay by addition of CTZ assay buffer containing native coelentrazine to the wells as described earlier. FIG. 19 shows that treatment with rituximab at both 1 μg/ml and 10 μg/ml concentrations resulted in increase in luciferase activity as compared to untreated cells, suggesting mild increase in cell death by treatment with rituximab alone. Addition of PBMC alone also resulted in a modest (approximately 3.5 fold) increase in GLuc luciferase activity. However, addition of rituximab in the presence of PBMC resulted in greater than 10-fold increase in GLuc activity as compared to the untreated cells. The result shows that the Matador cytotoxicity assay can be used to measure antibody dependent cellular toxicity in a rapid and sensitive manner. Essentially similar results were obtained when RAJI cells stably expressing NLuc were used.

### Example 21

### Use of Matador Assay to Measure Cytotoxicity Induced by a Bispecific Antibody

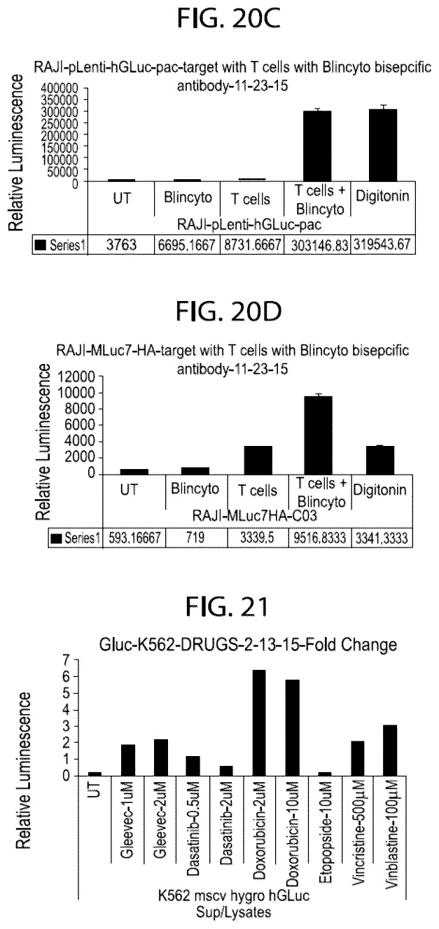

RAJI-MSCVhygro-hGLuc (FIG. 20A and FIG. 20B), RAJI-pLenti-Pac-T2A-hGLuc (FIG. 20C) and Plenti-MLuc7-MM-LL-HA (FIG. 20D) target cells were incubated with a bispecific antibody (Blinatumomab or BLINCYTO; AMGEN) at 100 ng/10⁶ cells for 30 min at 37° C. in PBS/2% FBS. Next, antibody-treated cells were washed three times in PBS, re-suspended in XVIVO medium and 100 μl (100K) cells were plated per well of a 96-well U-bottom plate. Effector cells (T-cells isolated by CD3 microbeads from Miltenyi Biotech) were washed twice in XVIVO medium and added to wells at an E:T ratio of 20:1. Total volume in each well was adjusted to 200 μl with medium. Plates were quickly centrifuged to settle cells, and incubated at 37° C. in a 5% CO₂ incubator for 4 hours. For maximum cell death, one hour prior to doing luciferase-based cell death assay, a total volume of 6 μl of Digitonin (stock 1 mg/ml) was added to wells containing target cells alone to achieve a final concentration of 30 g/ml of Digitonin. The plates were centrifuge at 1,000 rpm for 5 min and 50 μl of supernatants were transferred to a Greiner 384-well flat bottom white opaque plate. The luciferase activity was measured by BioTek synergy plate reader by directly injecting 0.5×CTZ assay buffer containing native coeloentrazine into the 384 well plates in a well mode. As shown in FIG. 20A, and FIG. 20C, treatment of GLuc expressing RAJI cells with T cells that had been incubated with Blinatumomab (BLINCYTO), resulted in significant increase in GLuc activity as compared to treatment with Blinatumomab or T cells alone. Essentially similar results were obtained when the experiment was repeated with RAJI cells expressing MLuc7-MM-LL-HA (FIG. 20D). The % specific lysis (FIG. 20B) was calculated by taking Digitonin-induced cell death as 100% and untreated cells as 0%. % specific lysis was calculated by using formula: % Specific lysis=100×((experimental data)−(spontaneous cell death))/((maximum cell death)−spontaneous cell death)).

### Example 22

### Use of Matadorassay for Measuring Cytotoxicity Induced by Targeted Agents and Chemotherapy Drugs

K562-MSCVhygro-GLuc cells were plated at a concentration of 100K cells/500 μl/well in a flat-bottom 24 well plate in phenol-red-free RPMI medium and treated with the indicated chemotherapeutic drugs. After 5 days of drug treatment, the cells were mixed well, collected in 1.5 ml microfuge tubes and divided into 3 groups of 166 μl each. For the first group, the cells with cell supernatant were directly assayed for luciferase activity by plating 50 μl of cells/well in a white 384 well plate in triplicate. For the second group, 166 μl of cells were centrifuged and only cell supernatants were collected in new tubes and plated at 50 μl/well in a white 384 well plate in triplicate. For the third group, samples were centrifuged, supernatant was discarded, cells were re-suspended in PBS, and 50 μl of re-suspended cells were plated per well in a white 384-well plate in triplicate. The luciferase activity in was precisely measured by BioTek synergy plate reader that directly injecting 0.5×CTZ assay buffer containing native coeloentrazine into each well of the 384 well plates in a well mode. Drug-induced cell death was measured by calculating fold-change by dividing mean luciferase values in cell supernatant (Group 2) by mean luciferase values in cell pellets (Group 3). Results are shown in FIG. 21 and demonstrate that GLuc assay can be used to measure cytotoxicity of targeted agents (e.g. Imatinib (or Gleevec) and Dasatinib) as well as conventional chemotherapy drugs (e.g. doxorubicin, vincristine and vinblastine).

### Example 23

### Development of Matador Assay Based on Transient Transfection of MLuc7, HT Luc, and Lucitia Luc

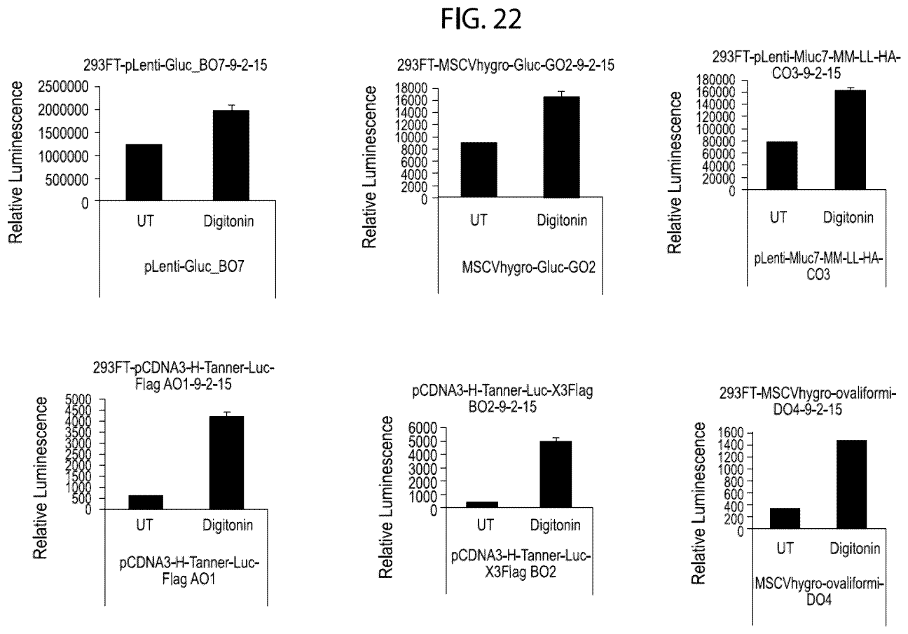

In addition to GLuc, a number of luciferases from the copepods, such as Pleuromamma abdominalis, Metridia pacifica, Metridia curticauda, Metridia asymmetrica, Metridia okhotensis, Metridia longa, Lucicutia ovaliformis, Heterorhabdus tanneri, and Pleuromamma scutullata have been described. To test if the other copepod luciferases can be used similarly to GLuc for development of an assay for cellular cytotoxicity, we cloned the cDNAs encoding the non-secretory (lacking the signal peptide) forms of Heterorhabdus tanneri luciferase (HtLuc; SEQ ID NO: 6), Lucicutia ovaliformis luciferase (LoLuc; SEQ ID NO: 5), and Metridia longa luciferase 7 (MLuc7; SEQ ID NO: 4) into mammalian expression vectors. The MLuc7 cDNA also carried M43L and M110L substitutions. The corresponding substitutions in GLuc have been previously shown to result in Glow type luminescence (Welsh, Patel et al. 2009). The mammalian expression vectors encoding the non-secretory forms of the above luciferase cDNAs were transiently transfected into 293FT cells by calcium phosphate co-precipitation method. Approximately 24 hours post-transfection, cells from each transfection were split into 12 wells of 384 well plates. 6 wells were treated with Digitonin and 6 wells were left untreated. Luciferase activity was measured by addition of coelentrazine containing assay buffer directly to each well. As shown in FIG. 22, treatment with Digitonin resulted in increase in luciferase activity in Digitonin-treated samples of 293FT cells transfected with all luciferases tested. These results demonstrate that, similar to GLuc, non-secretory forms of luciferases from other copepods can be used to develop assay for measurement of cell toxicity.

### Example 24

### Development of Matador Assay Based on Transient Transfection of GLuc, TurboLuc16, NLuc, Renilla, PaLuc1, HTluc1, LoLuc, Lucia Luc and MLuc7 in 293FT Cells

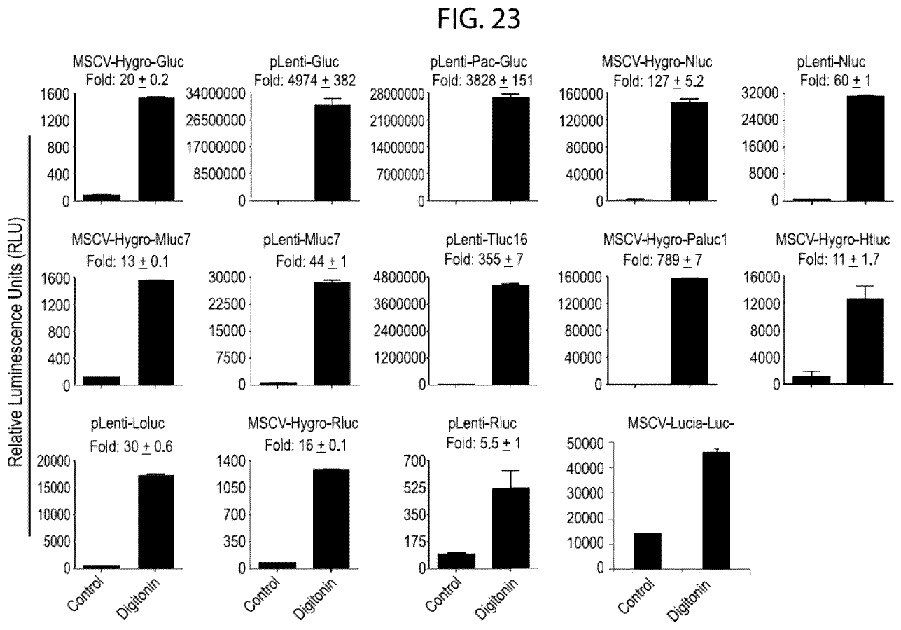

The indicated luciferase constructs in either retroviral or lentiviral vectors were transiently transfected into 293FT cells by calcium phosphate co-precipitation method. Approximately 18 hours post-transfection, cells were treated with Digitonin for 90 minutes or left untreated (control). Cell-free supernatants (25 μl) were assayed for luminescence by addition of coelenterazine containing assay buffer (25 μl) directly to each well in a 384-well lumitrac plate for GLuc, NLuc, MLuc7, TLuc16, Paluc1, Htluc1, Loluc and Renilla luc (RLuc). Essentially a similar procedure was used with a construct encoding Lucia-Luc. As shown in FIG. 23, treatment with Digitonin resulted in increase in luciferase activity in the cell-free supernatants of 293FT cells transiently transfected with all the luciferase constructs tested. Essentially similar results are obtained when the experiment is repeated using other marine luciferases listed in Table 1.

### Example 25

Use of Matador Assay when GLuc is Targeted to Cell Membrane

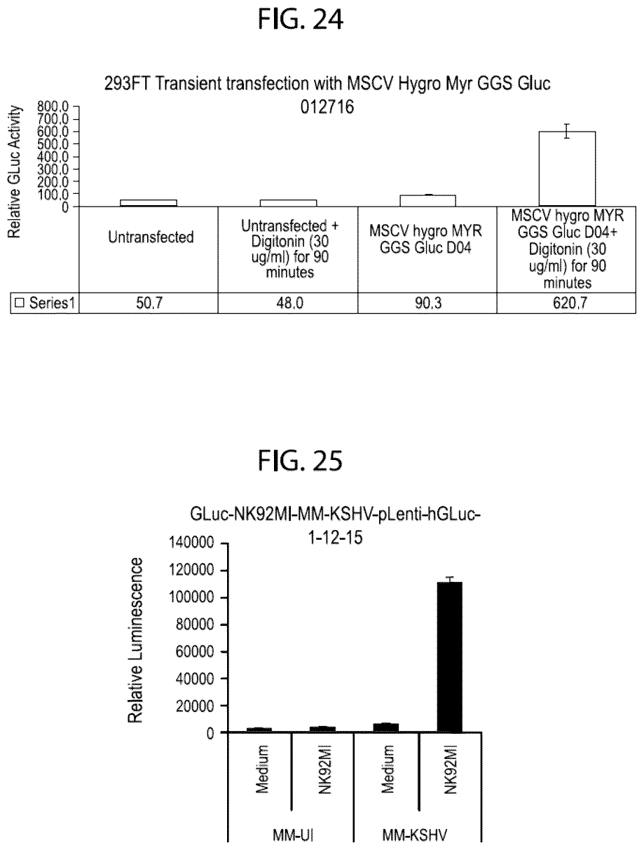

In the preceding examples, GLuc was expressed in the cytosol. To examine if the assay would work if GLuc is targeted to a specific cellular compartment, a construct (MSCV-hygro-MYR-GGS-GLuc-D04) was created in which GLuc encoding its amino acids 18 to 185 is expressed with an N-terminal Myristolyation sequence through an intervening Glycine-Glyceine-Serine linker. The GLuc cDNA in this construct also carried M60L and M127L mutations that have been previously shown to result in Glow type luminescence (Welsh, Patel et al. 2009). The sequence of the MYR-GGS-GLuc fragment is provided in SEQ ID NO: 30. 293FT cells were transiently transfected with the above construct or left untransfected. Approximately 18 hours post-transfection, cells were treated for 90 min with Digitonin (final concentration 30 μg/ml) or left untreated. After the 90 minute incubation, 200 μl supernatant was collected from each well in a 1.5 ml tubes. The tubes were spun down at1500 RPM for 5 minutes. Then, 25 μl supernatant was collected from the top and plated in a 384 well plate in triplicate (25 l/well). Luciferase activity was measured by addition of 0.5×CTZ assay buffer (25 l) directly to each well in well mode. FIG. 24 shows that treatment with Digitonin resulted in increase in Luc activity in case of 293FT cells transiently transfected with the MYR-GGS-GLuc construct. The fold increase in GLuc activity was however lower as compared to the experiments where GLuc was not targeted to the cell membrane through the myristoylation signal. Treatment with Digitonin also resulted in increase in GLuc activity in case of 293FT cells transiently transfected with the MYR-GGS-GLuc construct when measured in the cell plus supernatant fraction.

### Example 26

### Matador Cytotoxicity Assay can be Used to Measure Cytotoxicity Against Virally-Infected Cells

To determine if the reporter release assay can be used to measure cytotoxicity against virally infected cells, we tested the effector function of NK92MI cells against KSHV-transformed primary rat embryonic metanephric mesenchymal precursor (MM; a gift from Dr. S. J. Gao, University of Southern California) cells. MM-Parental and MM-KSHV cells were engineered to express GLuc by infection with pLenti-EF1α-Pac-T2A-Gluc-B07 (SEQ ID NO: 94) lentiviral vector and selection with puromycin. NK92MI effector cells were co-cultured with Gluc-expressing target cells at an E:T ratio of 05:1 co-culture for 4 hours in a 96-well flat bottom plate. The luciferase activity was precisely measured after directly injecting 0.5×CTZ assay buffer containing native coeloentrazine in the 96-well plate in well mode. FIG. 25 shows increase in GLuc activity upon co-culture of MM-KSHV-GLuc cells with NK92MI cells, indicating specific cytotoxicity. Thus, the Matador assay can be used to monitor cytotoxicity against virally infected cells. The above results also provide yet another example of the use of method of the Matador assay to measure cytotoxicity in solid-tumor derived adherent cells. They also demonstrate that the utility of the assay is not limited to human cells since MM cells are of rat origin.

### Example 27

### Uses of Matador Cytotoxicity Assay to Measure Cell Death Induced by Antibody Drug Conjugate Brentuximab Vedotin (Adcetris)

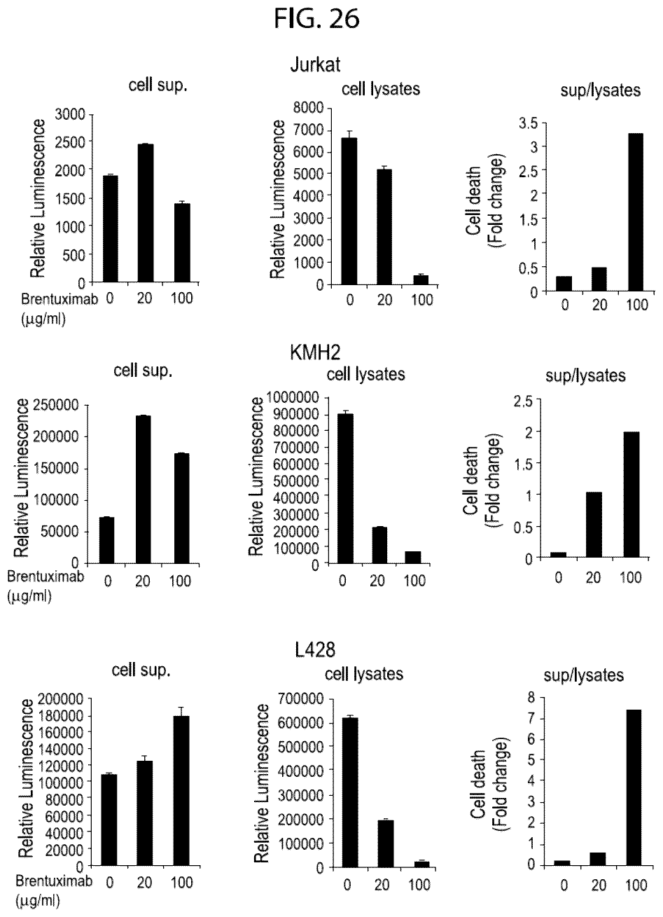

We next used the Matador cytotoxicity assay to measure cell death induced by CD30-directed antibody-drug-conjugate (ADC), Brentuximab vedotin (Adcetris). The CD30-positive Hodgkin lymphoma cells (L428 and KMH2) were engineered to express GLuc luciferase cDNA lacking the signal peptide by infecting these cells with pLenti-EF1α-Pac-T2A-Gluc-B07 (SEQ ID NO: 94) viral supernatant, followed by selection with puromycin to make stably transduced L428-GLuc, and KMH2-GLuc cells, respectively. Similarly, Jurkat cells were transduced with MSCVhygro-GLuc-HA-G02 (SEQ ID NO: 95) retroviral vector and selected with hygromycin to generate Jurkat-GLuc stable cell line. These GLuc-expressing target cells were plated at a concentration of 10×10⁵ cells/well in a flat-bottom 24 well plate in 500 μl of phenol-red-free RPMI medium with 10% FBS and treated with the indicated dose of Brentuximab vedotin. After 5 days of drug treatment, the cells were mixed well, collected in 1.5 ml microfuge tubes and divided into 3 groups of 166 μl each. For the first group, the cells with cell supernatant were directly assayed for luciferase activity by plating 30 μl of cells/well in a white 384 well plate in triplicate. For the second group, 166 μl of cells were centrifuged and only cell supernatants were collected in a new tube and plated at 30 μl/well in triplicate in a white 384 well plate. For the third group, samples were centrifuged, supernatant was removed, cells were lysed in Renilla lysis buffer (Promega) and 30 μl of cell lysates were plated per well in a 384-well plate in triplicate. The luciferase activity was precisely measured by BioTek synergy plate reader by injecting 0.5×CTZ assay buffer containing native coeloentrazine into each well of the 384 well plate in a well mode. As shown in FIG. 26, there was an increase in GLuc activity in the supernatant of Brentuximab vedotin-treated cells. However, in contrast to the cell-mediated cytotoxicity which occurs over a relatively short time (e.g., over 4-5 hour), Brentuximab vedotin-induced cell death is a slow process and takes 4-5 days to manifest. Since the duration of Brentuximab vedotin treatment was relatively long, the untreated cells had continued to proliferate as compared to the drug treated cells. As a result, at the time of GLuc assay, the number of GLuc-expressing cells in the wells that were left untreated was significantly higher as compared to the wells that were Brentuximab vedotin-treated. To control for the varying number of GLuc-expressing cells in different wells, which could have confounded the results of the Matador assay due to varying level of spontaneous GLuc-release, Brentuximab vedotin-induced cell death was measured by dividing mean GLuc values in the cell supernatant (Group 2) by mean luciferase values in cell lysates (Group 3). As shown in FIG. 26, Brentuximab vedotin treatment led to a significant increase in GLuc activity in cell supernatant when normalized for the GLuc activity in cell lysates (Right panels).

### Example 28

### Use of Matador Assay to Measure Activation Induced Cell Death

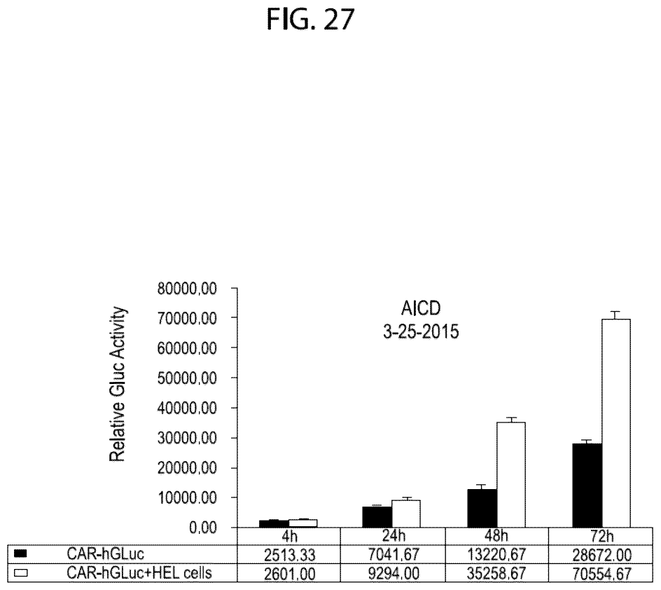

To determine the Activation Induced Cell death (AICD) in CAR-expressing primary cells, peripheral blood mononuclear cells (PBMCs) were obtained from healthy de-identified adult donors. PBMC were isolated from buffy coats by Ficoll-Hypaque gradient centrifugation and re-suspended in XVIVO medium (Lonza) supplanted with 10 ng/ml soluble anti-CD3, 10 ng/ml soluble anti-CD28 and 100 IU recombinant human-IL2. PBMCs were engineered to express a 2^{nd} generation chimeric antigen receptor (CAR) targeting a protein expressed on HEL.92.1.7 cells. The CAR construct consisted of human CD8 signal peptide, followed by codon-optimized sequences of a scFv derived from a monoclonal antibody directed against the extracellular domain of the target protein expressed on HEL.92.1.7 cells, a MYC epitope tag, the hinge, transmembrane and cytosolic signaling domains from human CD28 molecule, the cytoplasmic domain of human CD3ζ molecule, T2A ribosomal skip sequence and a Gaussia princeps luciferase cDNA sequence without the secretory signal. PBMC were transduced with CAR expressing lentiviral supernatant to make PBMC-CAR-GLuc effector cells. Specific activation-induced cell death of these GLuc expressing effector cells by HEL.92.1.7 cells was assayed after effector and target cells were co-cultured for 4 to 72 hours at an E:T ratio of 2:1 in a 384-well flat bottom plate. The luciferase activity was precisely measured by BioTek synergy plate reader that directly injected 15 μl of the 0.5×CTZ assay buffer into the 384 well plates in a well mode. As shown FIG. 27, there was a significant increase in GLuc activity when GLuc expressing CAR-T cells were co-cultured with the HEL target cells for 24 hours, 48 hours and 72 hours as compared to CAR-T cells that had been cultured alone without the target cells.

### Example 29

Development of Matador Assay Based on the Release of Enhanced Green Fluorescent Protein (EGFP) and mCherry

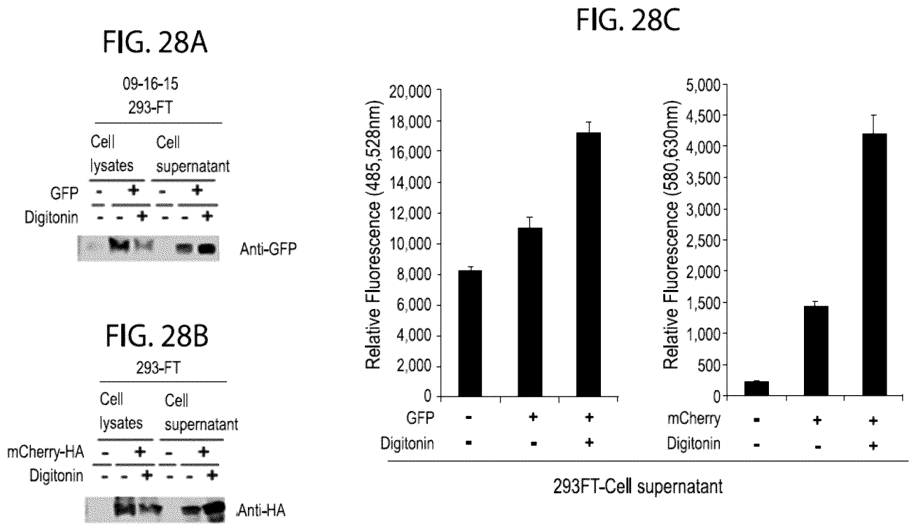

We next examined if the Matador cytotoxicity assay developed by us is limited to only luciferase proteins or can be adapted to any other protein that is not endogenously expressed by the target cells. For this purpose, we transiently transfected 293FT cells by calcium phosphate co-precipitation method with an expression plasmid encoding enhanced green fluorescent protein (GFP) or HA-epitope tagged-mCherry. Approximately 24 post-transfection, the transfected cells were either left untreated or treated with Digitonin to induce cell death. The cells were collected in tubes and centrifuged at 2,000 rpm for 5 min. Cell supernatant was collected in a new set of tubes and cell pellet were washed with cold phosphate-buffered saline twice and lysed in lysis buffer containing 20 mM Tris-HCl, pH 8.0, 500 mM NaCl, 0.25% Triton X-100, 1 mM EDTA, 10 mM β-glycophosphate, 10 mMNaF, 1 mM DTT, lx protease inhibitor mixture (Roche Molecular Biochemicals) at 4° C. for 30 min. After incubation, the mixture was pipetted five or six times to disperse the cells followed by centrifugation at 14,000 rpm at 4° C. for 10 min. The cell lysates were collected as whole cell extracts, and protein concentration was determined by using Bio-Rad protein assay reagent. 30 μl of cell supernatant or 30 g of whole cell extracts were heated in the presence of SDS-PAGE sample buffer and loaded on 10% SDS-PAGE gel followed by transferring to nitrocellulose membranes. These membranes were incubated with primary antibodies against specified proteins in Tris-buffered-saline with Tween 20 followed by incubation with secondary antibody (IRDye 800CW conjugates of goat anti-rabbit IgG; Li-Cor) and development with Odyssey Infrared Imaging System CLx (Li-Cor Biosciences). The secondary antibody was used at a 1:10,000 dilution. The blots were scanned and analyzed using an Odyssey Infrared scanner using Odyssey imaging software, version 2.0. The primary antibodies used in these experiments were anti-GFP or anti-HA polyclonal antibodies from Santa Cruz Biotechnology at 1:2,000 or 1:10,000 dilutions, respectively. As shown in FIG. 28A Digitonin-induced cell death of GFP transfected 293FT cells resulted in increase in GFP release in the supernatant fraction as measured by Western blotting with an antibody against GFP. There was a corresponding decrease in GFP expression in cell lysate fraction upon treatment with Digitonin. Similarly, Digitonin-induced cell death of HA-mCherry transfected 293FT cells resulted in increase in mCherry release in the supernatant fraction as measured by Western blotting with an antibody against HA tag (FIG. 28B). There was a corresponding decrease in mCherry expression in cell lysate fraction upon treatment with Digitonin. The above results demonstrate that any protein or peptide that can be expressed intracellularly and against which a suitable antibody or an antibody like binding-fragment is available can be used to develop an assay for cellular cytotoxicity. In addition, the results with mCherry-HA experiment demonstrate that an epitope tagged version of the protein can be used to develop an assay for cellular cytotoxicity. In addition to Western blotting, we used fluorescence measurement to detect release of GFP and mCherry in the supernatant fractions upon induction of cell death with Digitonin. Fluorescence signal from GFP (excitation=488 nm, emission=528 nm) and mCherry (excitation=580 nm, emission=630 nm) was measured using BioTek synergy plate reader. As shown in FIG. 28C, there was significant increase in fluorescence in cell supernatant collected from cells that had been transfected with GFP and mCherry-HA, respectively, and then treated with Digitonin as compared to untreated cells.

Taken collectively, these results demonstrate that a cytotoxicity assay can be developed using any molecule (e.g. protein, peptide, DNA, or RNA) that is 1) not expressed endogenously in the target cells; 2) can be ectopically expressed intracellularly; 3) is leaked extracellularly upon loss of cell membrane integrity; 4) can be measured in the extracellular compartment. The presence of protein/peptide can be detected by ELISA, mass-spectroscopy, Western blotting, or by measuring the activity of the protein by functional assays, such as luciferase reporter assay for luciferases or by measuring fluorescence as in case of a fluorescent protein, or combination thereof. In the case of DNA and RNA, various methods for measurement can be used, including PCR amplification, hybridization, mass spectroscopy, sequencing (including next generation sequencing) or combination thereof.

Finally, in another embodiment, it is possible to develop a cytotoxicity assay using 1) any molecule (e.g. protein, peptide, DNA, or RNA) that is not expressed endogenously in the cells; 2) can be ectopically expressed intracellularly; 3) whose detection requires the presence of factor or factors that are not cell membrane permeable or are cell membrane permeable to only limited extent and therefore are excluded from the intracellular compartment; 4) where the above factor or factors gain increased entrance to the intracellular compartment upon loss of cell membrane integrity which results in enhanced detection of the molecule described in step 1 that is ectopically expressed intracellularly. In the case of DNA and RNA, various methods for measurement can be used, including PCR amplification, hybridization, sequencing (including next generation sequencing) or combination thereof.

### Example 30

### Use of Matadaor Assay Based on Calculation of % Specific Lysis

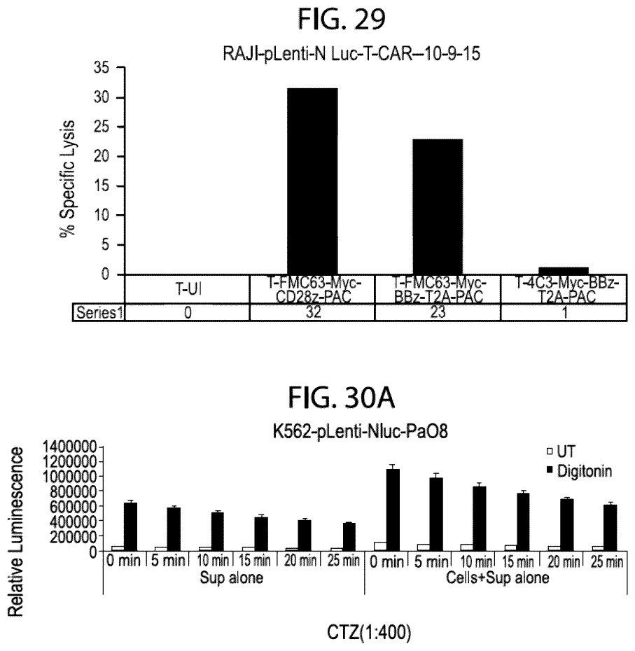

We constructed two lentiviral vectors encoding second generation CD19-specific CARs designated FMC63-Myc-BBz-T2A-Pac (SEQ ID NO: 80) and FMC63-Myc-28z-T2A-PAC (SEQ ID NO: 81), respectively, and a control CAR designated 4C3-Myc-BBz-T2A-PAC (SEQ ID NO: 83). To generate T cells expressing CD19-specific CAR, T cells were transduced by CAR lentiviral supernatant by spinfection at 2,800 rpm at 32° C. for 90 min. The spinfection steps were repeated for three consecutive days and cells selected in puromycin and expanded. The target (RAJI-NLuc) cells were plated a concentration of 5×10⁴ cells/well in a white 384-cell plate and either left untreated or treated with T cells expressing the CARs FMC63-Myc-BBZ-T2A-PAC, FMC63-Myc-CD28z-T2A-PAC or 4C3-Myc-BBz-T2A-PAC at an E:T ratio of 10:1 for 4 h. For measurement of maximum cell death, cells were treated with 30 μl of Digitonin dissolved in DMSO for approximately 90 min prior to measurement of NLuc activity. The cell death was assayed by luciferase-based cytotoxicity assay as discussed earlier by addition of 0.5×CTZ assay (15 μl/well) buffer containing native coelentrazine using an automatic injector. The % specific lysis was calculated by taking Digitonin-induced cell death as 100% and untreated cells as 0%. % specific lysis was calculated by using formula: % Specific lysis=100×((experimental data)−(spontaneous cell death))/((maximum cell death)-spontaneous cell death)). FIG. 29 shows increase in % specific lysis of RAJI-NLuc cells when co-cultured with CD19-directed FMC63-Myc-BBZ and FMC63-Myc-CD28z CAR constructs.

### Example 31

Effect of Different Assay Buffers on Glow-Type Luminescence by NLuc when Used in Matador Assay

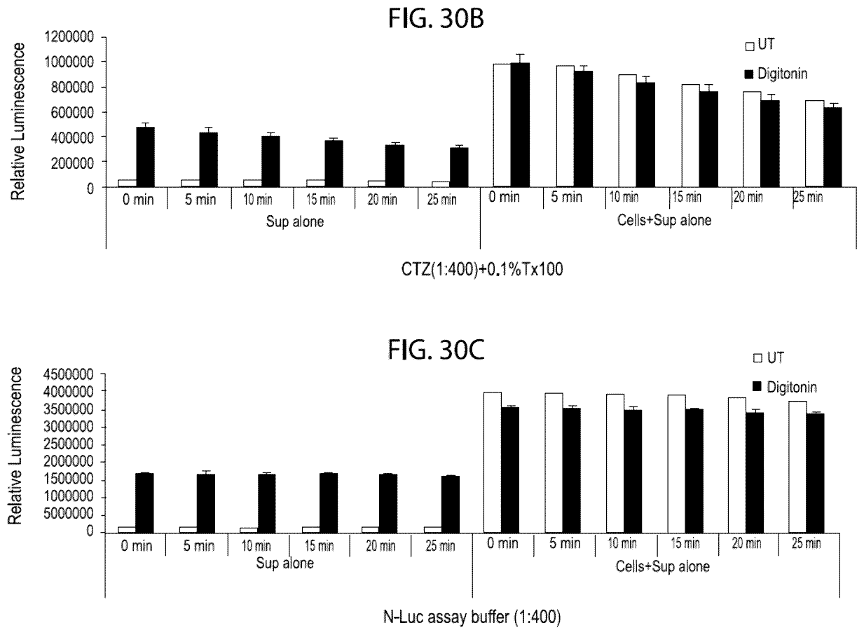

NLuc is known to result in glow-type luminescence which makes it an attractive candidate for inclusion in assays that are high-throughput compatible without the need for an automatic injector. To examine if NLuc would also result in glow-type luminescence when used in a cytotoxicity assay, K562-NLuc cells (1 million cells/ml) in XVIVO medium were aliquoted into 4 tubes. Cells in tubes 1 and 3 were left untreated while those in the tubes 2 and 4 were treated with Digitonin (30 μg/ml) for 1 h at 37° C. in duplicate. Tubes 1 and 2 were centrifuged to collect supernatant which were transferred into new tubes. Tubes 3 and 4 were designated as cells plus supernatant samples. All samples were diluted 1:2 in XVIVO medium and assayed in 384-well white plates. 25 μl of each sample was plated in each well and assayed using three assay buffers: A) CTZ diluted 1:400 in PBS; B) CTZ diluted 1:400 in PBS with 0.1% Triton 100; C) Nano-Glo® luciferase assay system (Promega) where Nano-Glo® assay substrate was diluted (1:400) in 1× Nano-Glo® assay buffer provided with the kit. 25 μl of assay buffer (A, B or C) was added in each well (n=5 for each sample) manually. Luminescence was read in a kinetic mode after 0, 5, 10, 15, 20 and 25 minutes. FIG. 30 shows that there is increase in luminescence upon Digitonin treatment when measured in the supernatant using all three assay buffers. However, while the luminescence value was stable in samples for over 25 minutes after addition of buffer C, it started declining within 5 minutes after addition of buffers A and B. These results demonstrate that the NLuc cytotoxicity assay results in a glow-type luminescence when performed on the cell supernatants using buffer C (i.e. Nano-Glo® assay reagent; Promega), making the assay high-throughput compatible without the need for an automatic injector. FIG. 30 further shows that there is increase in luminescence upon Digitonin treatment in the cells plus supernatant samples only in the buffer A (i.e. CTZ diluted 1:400 in PBS). No increase in luminescence upon Digitonin treatment in the cells+supernatant samples was observed in assay buffers B and C as the luminescence value was very high even in wells without Digitonin treatment. This is probably due to induction of cell death by 0.1% Triton X in case of buffer B. The exact composition of Nano-Glo® Luciferase Assay Buffer is proprietary but it could contain a cell death-inducing substance similar to Triton X. These results demonstrate that the Nano-Glo® assay reagent is not ideally suited for use in the Matador cytotoxicity when the assay is performed in a homogenous manner (i.e. performed on cells plus supernatant). It is, however, possible to use the Nano-Glo® assay reagent in the Matador assay when the assay is performed on cell supernatants that are freed from the cells. As it was suspected that the Nano-Glo® Luciferase assay buffer contains a cell death inducing agent, it was next examined if the Nano-Glo® substrate (Promega) could be diluted in Phosphate Buffer Saline (PBS) for use in the Matador assay. The experiment was repeated with RAJI-NLuc cells using Nano-Glo® substrate diluted 1:400 in PBS. It was observed that Nano-Glo® substrate in PBS led to glow-type luminescence with signal that was stable for 20-25 minutes. Additionally, in contrast to the situation with Nano-Glo® reagent (i.e. Nano-Glo® substrate dissolved in the Nano-Glo® Luciferase assay buffer), there was a clear difference in the signal between the untreated and digitonin treated cell samples when Nano-Glo® substrate suspended in PBS was used in the assay. These results demonstrate that Matador assay can be performed in homogeneous manner with NLuc expressing cells using Nano-Glo® substrate suspended in PBS. It was also noted that addition of Nano-Glo® reagent to K562-NLuc cells using an autoinjector followed immediately (i.e. without any time delay) by the measurement of luminescence still resulted in an increase in NLuc activity as compared to untreated cells. This result suggested that the exposure of the target cells to the cell death inducing agent present in the Nano-Glo® can instantly lead to induction of cell death, which can be measured by the Matador assay. Hence, depending on the agent that is being tested, the Matador assay can be performed even when the duration of exposure of the target cells to the test agent is very short (e.g., a second or less).

### Example 32

### Effect of Bovine Serum Albumin on Generation of Glow-Type Luminescence in Matador Cytotoxicity Assay

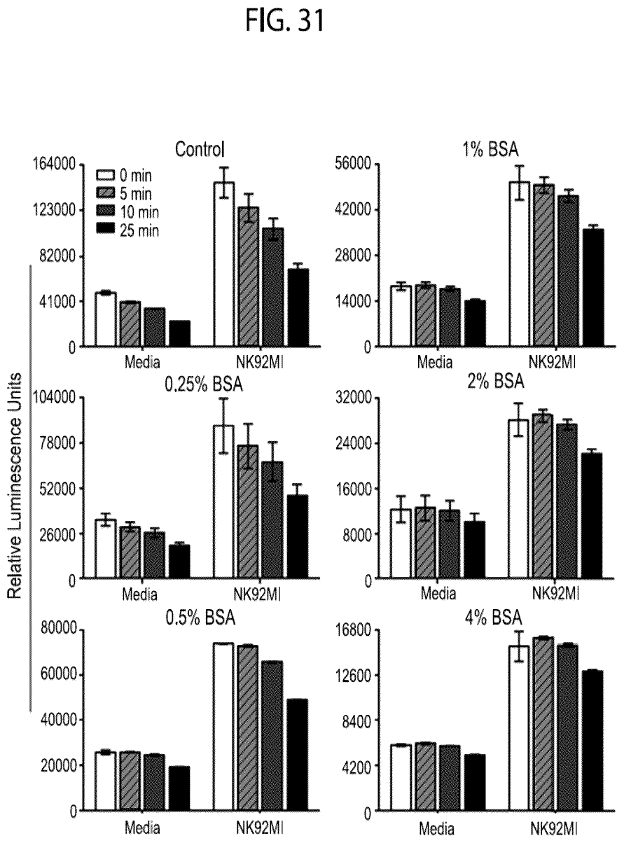

As addition of bovine serum albumin (BSA) has been reported to stabilize the luminescence, various concentrations of bovine serum albumin (BSA) were added in the assay buffer in order to make the assay high-throughput compatible in a homogeneous setting (i.e. when performed on cells +supernatant). K562 cells stably expressing NLuc were plated in a 384-well plate (15000 cells/15 μL) and treated with media alone or co-cultured with NK92MI at Effector:Target (E:T) ratio of 1:1 for 4 hours. After the incubation, the luminescence was measured from whole homogeneous mixture by manually adding CTZ containing assay buffer with indicated concentrations of BSA using a multi-channel pipette, read immediately (0 min) and at the indicated time points. The values shown are mean±SE of a representative experiment performed in duplicate for at least two times. FIG. 31 shows that addition of BSA to the CTZ assay buffer produced glow-type luminescence with stabilization of NLuc luminescence signal starting at a BSA concentration of >0.5%. These results demonstrate that by modifying the assay buffer with appropriate stabilizing agents the Matador cytotoxicity assay can be made high-throughput compatible in homogeneous setting when the assay buffer is added manually thus obviating the need of a luminometer equipped with an auto-injector.

### Example 33

### Matador Assay can be Used to Monitor Cell Toxicity In Vivo

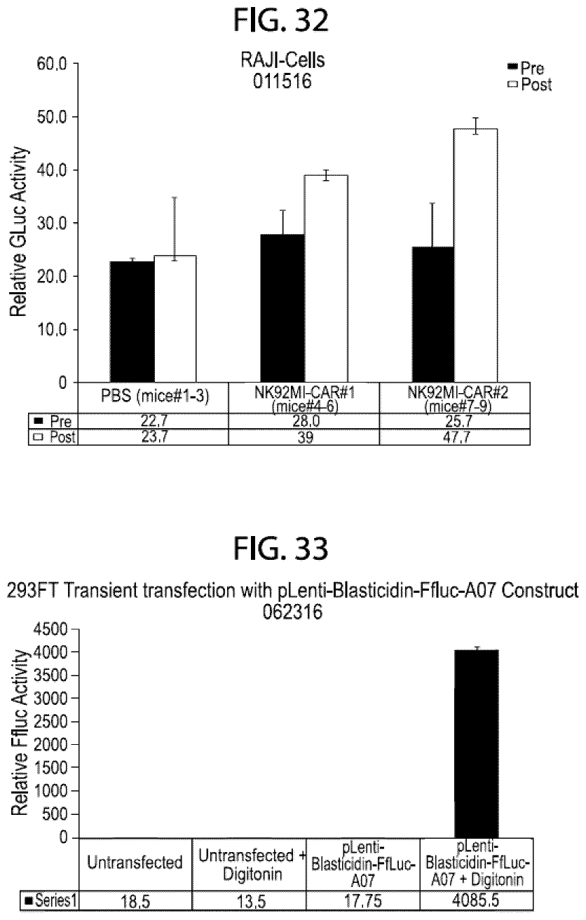

Raji cells were engineered to co-express GLuc and FfLuc (firefly luciferase) by sequential infection with lentiviral vectors (pLenti-EF1α-Pac-T2A-GLuc-B07 and pLENTI-EF1-pGL3-FfLuc-blasticidin-A07) expressing the two luciferases and selection with Puromycin and Blasticidin, respectively. However, only the GLuc activity was measured in this assay. Approximately 8 weeks old male NSG mice (n=9; Jackson Lab) were numbered 1 through 9 and injected with approximately 2 million Raji-GLuc/FfLuc cells in 100 μl PBS. Three additional mice (#10-12) were not injected with Raji cells and served as blanks. Approximately 30 minutes later, 50-100 μl of blood (pre-treatment sample) was collected from retro-orbital plexus of all animals (including blank animals) and immediately placed on ice. Approximately 1 hour post-injection of Raji cells, animals #1-3 were injected with 1001 of PBS while animals #4-6 and #7-9 were injected with 1001 of PBS containing 2 million of NK92MI cells expressing CAR #1 (chimeric antigen receptor #1) and CAR #2 (chimeric antigen receptor #2). 4 hours post NK92MI cells injection, 50-100 μl of blood (post-treatment sample) was collected from retro-orbital plexus of the mice and immediately placed on ice. The samples were spun at 4000 g for 10 min at 4° C. to separate serum. The serum was diluted 5 fold (i.e. 6 μl serum+24 μl PBS). GLuc activity in the diluted samples (25 l) was assayed using BioTek synergy plate reader that directly injected 25 μl of the 0.5×CTZ assay buffer into the samples plated in a 384 well plate as described earlier. The averaged value of blanked samples was subtracted from all test values to control for non-specific background reading. FIG. 32 shows that there was no significant increase in GLuc activity between the pre- and post-treatment samples of mice that had been treated with PBS. However, there was a significant increase in GLuc activity between the pre- and post-treatment samples of mice that had been treated with NK92MI cells expressing the two different chimeric antigen receptors. These results demonstrate that the Matador assay described in this invention can be used to monitor cytotoxicity in vivo.

### Example 34

### Development of Matador Assay Based on Transient Transfection of FfLuc in 293FT Cells

The mammalian expression vectors encoding Firefly luciferase cDNA (pLENTI-Blasticidin-FfLuc-A07; SEQ ID NO: 99) was transiently transfected into 293FT cells by calcium phosphate co-precipitation method. Approximately 18 hours post-transfection, one well for each transfection was treated for 90 min with Digitonin (final concentration 30 μg/ml) and the other well was left untreated. After the 90 minute incubation, 200 μl of supernatant was collected from each well in 1.5 ml tubes. The tubes were spun down at 1500 RPM for 5 minutes. Then, 25 μl of supernatant was collected and plated in a 384 well plate in triplicate (25 μl/well). Luciferase activity was measured in well mode using BioTek synergy plate reader by addition to each well of 25 μl Luciferase assay buffer described in FfLuc Assay, which consisted per 1.0 ml of 885.5 μl assay buffer+1 μl DTT (1M stock)+100 μl of 10× Luciferin stock solution+13.5 μl ATP (100 mM stock). As shown in FIG. 33, treatment with Digitonin resulted in increase in luciferase activity in case of 293FT cells transiently transfected with the FfLuc construct.

### Example 35

Development of Dual Reporter Based Matador Assay Utilizing a Beetle Luciferase (e.g. FfLuc) and a Marine Luciferase (e.g. GLuc)

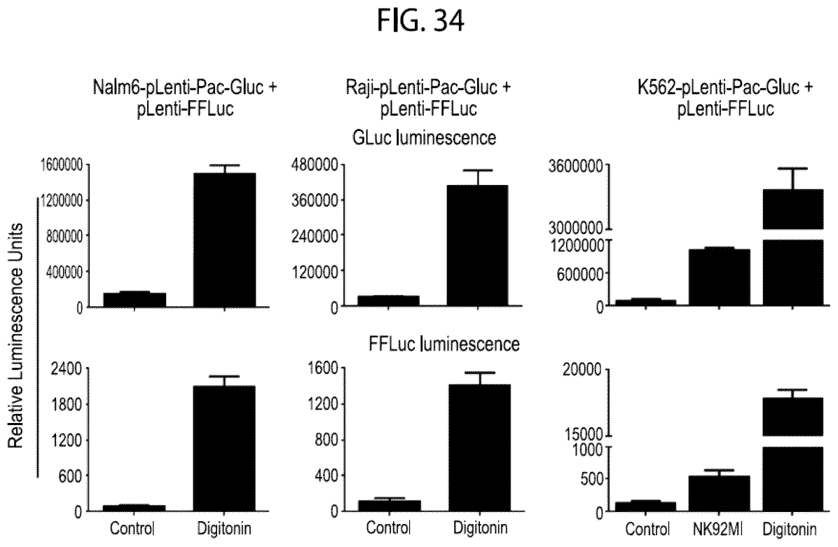

FfLuc and GLuc utilize different substrates (i.e., D-luciferin and coelentrazine) for light production. It was therefore next checked if increase in the activity of both GLuc and FfLuc can be measured from cells expressing both luciferases as a measure of cytotoxicity. For this purpose, Nalm6, Raji and K562 cells were engineered to stably express both GLuc and FfLuc by sequential infection with lentiviral vectors encoding the respective luciferases followed by selection with appropriate Puromycin and Blasticidin. Briefly, 20000 cells in 20 μl media were plated in each well of a 384 well plate followed by addition of 20 μl of Digitonin (final concentration 30 μg/ml) or media alone (20 μl) and incubated for 90 minutes. In the case of K562 cells, cell death was also induced by coincubation with NK92MI cells for 4 hours at an E:T ratio of 1:1. After the incubation, the firefly and GLuc activities were sequentially measured from the sample using the Dual-Luciferase® Reporter (DLR™) Assay System (Promega) and following the manufacturer's recommendations. As shown in the FIG. 34, treatment with Digitonin resulted in increase in both FfLuc and GLuc activities. Co-culture of K562 cells with NK92Mi cells also resulted in increase in both FfLuc and GLuc activities. Essentially, similar results are obtained when the assay is repeated using cells expressing other beetle luciferases in combination with different marine luciferases (e.g. NLuc). Thus, surprisingly and in contrast to a previous report (Fu, Tao et al. 2010), we were able to successfully detect significant increase in FfLuc activity upon induction of cell death from cells stably expressing this reporter in the cytosol, suggesting that FfLuc and other beetle luciferases can be potentially used in Matador cytotoxicity assay. It is possible that the success of our assay is in part due to use of optimized vectors, such as lentiviral vectors, and use of strong promoters (e.g., CMV and EF1α promoters) that result in higher levels of FfLuc expression.

### Example 36

### Stability of Different Luciferases Under the Conditions of Matador Assay

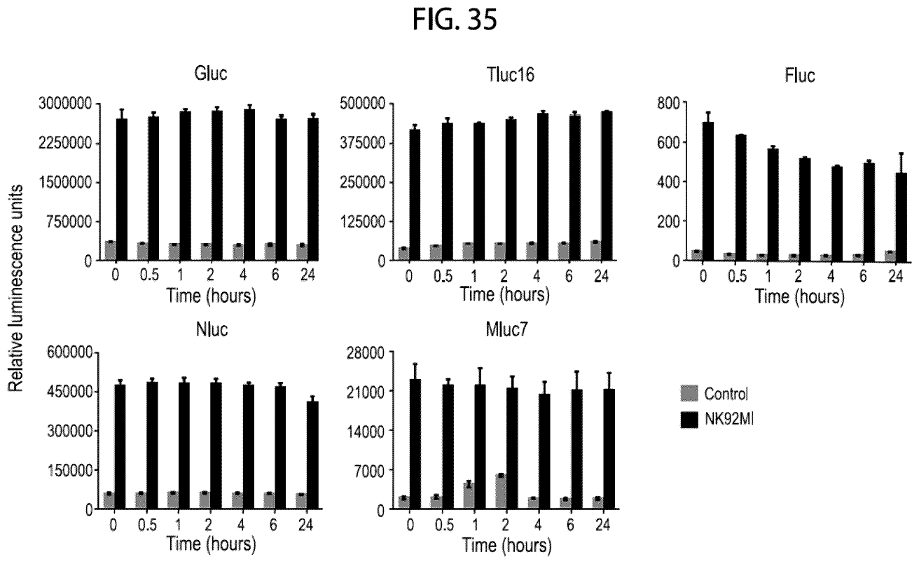

The shorter half-life (<30 minutes) of firefly luciferase (FLuc or FfLuc) in tissue culture medium has been reported to make it impractical to use it in a standard 4 hours co-culture cytotoxicity assay (Thompson, Hayes et al. 1991, Schafer, Schafer et al. 1997). Although the marine luciferases used in this study have been reported to have longer half-life (>24 hours) in tissue culture medium, we wanted to confirm that they would retain their stability under the assay conditions as the cell death proteases released during cell death could lead to their degradation. Therefore, to demonstrate that the marine luciferases are suitable for the Matador cytotoxicity assay, K562 cells stably expressing GLuc, NLuc, TLuc16, Mluc7 and Fluc, were plated in a 24-well plate and treated with media alone or with NK92MI cells at an Effector:Target (E:T) ratio of 1:1 for 6 hours. Post-incubation, the cell-free supernatants were divided into 7 different tubes and frozen immediately at −80° C. The tubes were then incubated at 37° C. for 0, 0.5, 1, 2, 4, 6, and 24 hours. After incubation, supernatants were transferred to a 384-well plate, and luminescence was measured by adding CTZ-containing assay buffer (for GLuc, NLuc, TLuc16, and Mluc7), and D-luciferin-containing assay buffer (for Fluc), respectively. As shown in FIG. 35, luminescence of GLuc, NLuc, TLuc16, and Mluc7 was stable for 24 hours at 37° C. in cell culture supernatants thereby demonstrating the suitability of these luciferases for the cytotoxicity assay. In contrast, luminescence of Flue started decaying as early as 0.5 hours after incubation at 37° C. in cell culture supernatants, confirming that Fluc may not be ideal for the Matador cytotoxicity assay.

### Example 37

Use of Thermostable Luciferase LucPPe-146-1H2 from Photuris Pennsylvanica in Cytotoxicity Assay

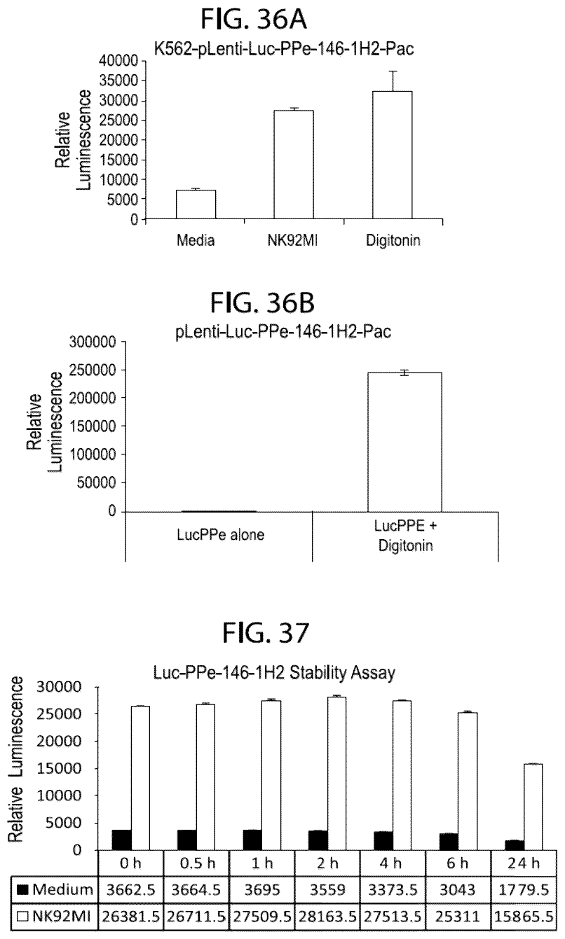

It was conceivable that short half-life of FLuc under the conditions of Matador assay is due to its poor thermostability. Thermostable luciferases from Photuris pennsylvanica and Pyrophorus plagiothalamus have been described (PCT/US99/30925). To examine whether thermostable luciferases are suitable for Matador cytotoxicity assay, K562 cells stably expressing LucPPe-146-1H2 mutant (SEQ ID NO: 21) were plated in triplicate at approximately 20,000 cells/well in a Greiner 384-well flat bottom white opaque plate. The cells were cultured alone or with NK92MI cells at an E:T ratio of 1:1 for 4 hours in 401 total volume. For measurement of maximum cell death, cells in three wells were treated with Digitonin (final concentration 30 μg/ml). At the end of co-culture period, the luminescence was measured by adding D-luciferin containing assay buffer (40 μl) to the cell-culture medium containing the cells at 1:1 (v/v) ratio. FIG. 36A shows that induction of cell death of K562 cells stably expressing the LucPPe-146-1H2 by Digitonin or by co-culture with NK92MI cells results in increase in LucPPe-146-1H2 activity. FIG. 36B shows that transient transfection of LucPPe-146-1H2 in 293FT cells also resulted in increase in LucPPe luciferase activity in Digitonin treated cells as compared to cells that had not been treated with Digitonin. The above results demonstrate that the activity of the thermostable beetle luciferases, such as LucPPe-146-1H2, is increased upon induction of cell death and therefore they are suitable candidates for inclusion in the Matador cytotoxicity assay.

### Example 38

Stability of Thermostable Luciferase LucPPe-146-1H2 from Photuris Pennsylvanica Under Conditions of Cytotoxicity Assay

Although thermostable luciferases are stable at 37° C., it was not obvious whether thermostability is the only criteria for stability of a reporter under the conditions used in the cytotoxicity assays. Thus, a thermostable luciferase could be degraded under the culture conditions of the cytotoxicity assay due to the activity of proteases that are present in the serum or are released during the process of cell death. Therefore, to examine whether thermostable luciferases are suitable for inclusion in the Matador cytotoxicity assay, K562 cells stably expressing LucPPe-146-1H2 mutant were plated in a 6-well plate, treated with media alone (control) or co-cultured with NK92MI at Effector:Target (E:T) ratio of 1:1 for 4 hours. After incubation, cell-free supernatants were transferred into 7 different tubes and frozen immediately at −80° C. The tubes were directly transferred to 37° C. (from −80° C.) and were incubated for indicated time periods (0 to 24 hours). The luminescence was measured by adding D-luciferin containing assay buffer directly to each well in well mode. FIG. 37 shows increase in LucPPe-146-1H2 luminescence in the supernatants collected from cells that had been co-culture with NK92MI cells. Furthermore, the luminescence signal was stable in the cell culture medium for nearly 4-6 hours. These results demonstrate that thermos-stability is a key determinant of stability of reporters under the conditions of use in the cytotoxicity assays. As most cytotoxicity assays are carried out at 37° C., these results demonstrate that thermostable reporters are the preferred reporters to be used in the cytotoxicity assay described in this invention. In addition to LucPPe-146-1H2, GLuc, NLuc and TLuc, a number of other thermostable reporters have been described in the art (e.g., PCT/US99/30925) and are the preferred reporters for use in the cytotoxicity assay of the present invention.

### Example 39

### Development of Dual Luciferase Based Matador Assay Using LucPPe-146-1H2 and NLuc

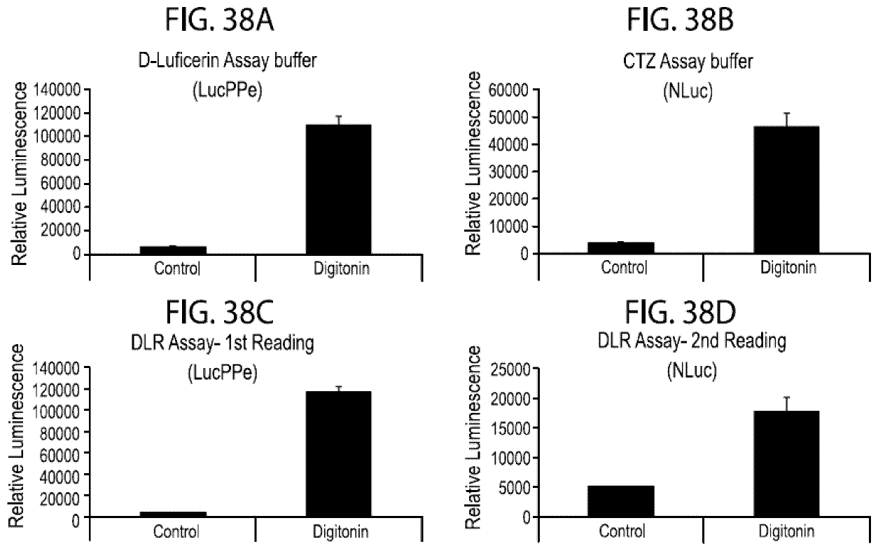

It was next checked if increase in the activity of both NLuc and LucPPe-146-1H2 can be measured from cells expressing both luciferases as a measure of cytotoxicity. For this purpose, K562 cells were engineered to stably express both NLuc and LucPPe-146-1H2 by sequential infection with lentiviral vectors (pLENTI-NLuc-AcV5-Blasticidin-Pa08, SEQ ID NO: 92 and pLenti-EF1-LucPPe-146-1H2-Flag-Pac-RO1; SEQ ID NO: 100) encoding the respective luciferases followed by selection with Blasticidin and Puromycin, respectively. Briefly, 20000 cells in 20 μl media were plated in each well of a 384 well plate followed by addition of 20 μl of Digitonin (final concentration 30 μg/ml) or media alone (20 μl) and incubated for 90 minutes. After incubation, the LucPPe-146-1H2 and NLuc activities were sequentially measured from the sample using the Dual-Luciferase® Reporter (DLR™) Assay System (Promega) and following the manufacturer's recommendations. Essentially, 20 μl of FFluc luciferase assay buffer was first added and the resulting luminescence detected for LucPPe-146-1H2. Then 20 μl of Stop and Glo assay buffer was added to the same wells and the resulting luminescence was measured for NLuc. FIG. 38A-B show that treatment with Digitonin resulted in increase in both LucPPe-146-1H2 and NLuc luminescence in cells co-expressing both luciferases when measured using the Dual-Luciferase® Reporter (DLR™) Assay System. The LucPPe-146-1H2 and NLuc activities were also measured from the samples that had been added to different wells. For this purpose, LucPPe-146-1H2 was measured by addition of 40 μl of D-luciferin assay buffer to three wells containing supernatant from untreated cells and three wells containing supernatant from Digitonin-treated cells, respectively. Similarly, NLuc activity was measured by addition of 40 μl of CTZ assay buffer to three wells containing supernatant from untreated cells and three wells containing supernatant from Digitonin-treated cells, respectively. FIG. 38C-D show that treatment with Digitonin resulted in increase in both LucPPe-146-1H2 and NLuc activities in cells co-expressing both luciferases. While in the above examples, a single cell co-expressed both beetle (i.e. LucPPe-146-1H2) and marine (NLuc) luciferases, it is possible to conduct the assay in which one target cell expresses one luciferase (e.g. LucPPe-146-1H2) and the other target cell expresses the other luciferase (e.g. NLuc). The two cells are mixed together in the same well and exposed to the cytotoxic agent. Digitonin-treated cells are used as a measure of maximum cell death for both cell types. Subsequently, the relative effect of the cytotoxicity agent on the two cell types is measured by assaying for the LucPPe-146-1H2 and NLuc activities, respectively.

In the above examples, the experiment is conducted with LucPPe-146-1H2 and NLuc. Essentially similar results are obtained when the assay is repeated using cells expressing other thermostable beetle luciferases in combination with different marine luciferases (e.g. GLuc) or RLuc.

### Example 40

Use of Thermostable Luciferases from Photuris pennsylvanica and Pyrophorus plagiothalamus in Matador Cytotoxicity Assay

Many thermostable luciferases from Photuris Pennsylvanica and Pyrophorus Plagiothalamus have been described (PCT/US99/30925). To examine whether other thermostable luciferases are suitable for cytotoxicity assay, K562 cells stably expressing Luc49-7C6A, Luc78-0B10, Luc133-IB2 and LucPpL-81-6G1 are plated in triplicate at approximately 20,000 cells/well in a Greiner 384-well flat bottom white opaque plate. The cells are cultured alone or with NK92MI cells at an E:T ratio of 1:1 for 4 hours in 40 μl total volume. For measurement of maximum cell death, cells in three wells are treated with Digitonin (final concentration 30 μg/ml). At the end of co-culture period, the luminescence is measured by adding D-luciferin containing assay buffer (40 l) to the cell-culture medium at 1:1 (v/v) ratio in well mode. Induction of cell death by Digitonin and NK92MI cells is shown to result in increase in Luc49-7C6A, Luc78-0B10, Luc133-IB2 and LucPpL-81-6G1 activities as compared to the activities measured in untreated samples.

### Example 41

### Utility of the Matador Cytotoxicity Assay to Demonstrate Specificity of a Targeting Agent and to Rule Out Non-Specific Toxicity

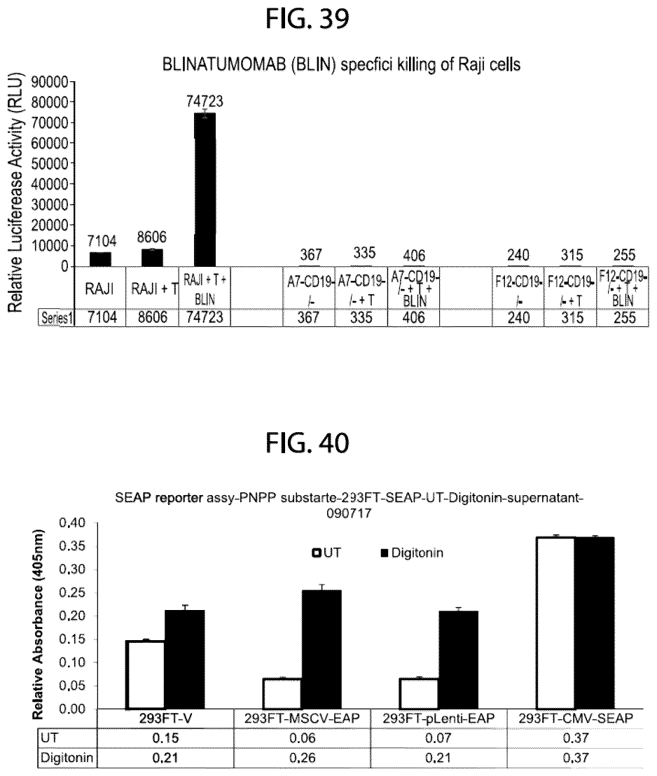

The off-target toxicity of immune-oncology agents, such as bispecific antibodies and CAR-T cells, is a major concern during their research and development. To demonstrate that the Matadaor cytotoxicity assay can be used to rule out off-target toxicity, RAJI cells were stably infected with a retroviral vector encoding a Cas9 targeting human CD19. Two clones of RAJI cells lacking CD19 expression (A7/CD19^{-/-} and F12/CD19^{-/-}) as determined by immunofluorescence staining with a CD19 antibody were selected by flow cytometry and were subsequently stably transduced with a vector encoding NLuc. Primary T cells were incubated with Blinatumomb (BLIN) at a final concentration of (100 ng/1 million T cells) for 30 mins at 4° C. RAJI/NLuc, A7/CD19^{-/-}/NLuc and F12/CD19^{-/-}/NLuc (5×10⁴ cells/30 μl) were incubated with T cells or with T cells that had been pre-incubated with Blinatumomb (3×10⁵ cells/30 μl) or with medium for 4 hours (37° C. at 5% CO₂) in white 384 well plates. Cell death was assessed by injecting 30 μl of CTZ-containing assay buffer using an auto-injector to measure luminescence. FIG. 39 shows that co-culture of RAJI/NLuc cells with T cells plus Blinatumomab results in significant increase in NLuc activity as compared to co-culture with unmodified T cells while co-culture of T cells plus Blinatumomab with A7/CD19^{-/-}/NLuc and F12/CD19^{-/-}/NLuc cells failed to do so. These results show that cytotoxicity of Blinatumomab is specific for CD19-expressing cells, thereby demonstrating that the Matador cytotoxicity assay can be used to rule out off-target toxicity of an agent.

### Example 42

### Utility of the Matador Cytotoxicity Assay to Demonstrate Specificity of a Targeting Agent and to Rule Out Non-Specific Toxicity

It is next examined if the positive and negative control target cell lines can be combined in a single well for the performance of the Matador cytotoxicity assay, which would result in reduced cost and improved efficiency. Approximately 1×10⁵ RAJI/NLuc and A7/CD19^{-/-}/NLuc cells are mixed separately with equal number of RAJI/LucPPe-146-1H2 cells. To demonstrate the specificity of the assay, 1×10⁴ RAJI/NLuc cells plus 1×10⁴ RAJI/LucPPe-146-1H2 cells or 1×10⁴ A7/CD19^{-/-}/NLuc plus 1×10⁴ RAJI/LucPPe-146-1H2 cells are co-cultured in triplicate with unmodified T cells or T cells expressing a CD19-CAR at an E:T ratio of 10:1 in a 384 well plate. After 4 hour, cultures are assayed for NLuc and LucPPe-146-1H2 activities using CTZ and D-Luciferin assay buffers respectively or using the Nano-Glo® Dual-Luciferase® Reporter (NanoDLR™) assay (Promega) and following the instructions of the manufacturer. Co-culture with CD19-CAR-T cells is shown to result in increase in both NLuc and LucPPe-146-1H2 activities in the wells containing a mixture of RAJI/NLuc plus RAJI/LucPPe-146-1H2 cells as compared with co-culture with unmodified T cells. However, co-culture with CD19-CAR-T cells is shown to result in an increase in only LucPPe-146-1H2 activity in the wells containing a mixture of RAJI/CD19^{-/-}/NLuc plus A7/LucPPe-146-1H2 cells as compared with co-culture with unmodified T cells. Essentially a similar approach is used to generate NLuc-expressing RAJI/CD20^{-/-}, RAJI/CD22^{-/-}, NALM6/CD20^{-/-}, NALM6/CD22^{-/-}, U266/BCMA^{-/-}, L363/BCMA^{-/-}, BV173/CD123^{-/-}, MV:411/CD123^{-/-}, HL60/CD33^{-/-}, MG-63/IL13RA2^{-/-}, Jurkat/CD30^{-/-}, and HEL-92.1.7/MPL^{-/-} cell lines. The resulting gene knock out cells expressing NLuc and their wild-type counterparts expressing LucPPe-146-1H2 are used to determine the on-target and off-target cytotoxicity of chimeric antigen receptors and bispecific antibodies targeting the corresponding target antigens. In another exemplary embodiment, expression of B2 microglobulin (B2M) is knocked-down by Cas9 approach in the HLA-A2+ BV173 cell line. As B2M is needed for HLA-A2 expression, this results in the loss of HLA-A2 expression. The BV173/B2^{-/-}/NLuc cells are used to monitor the on-target and off-target cytotoxicity of T cells expressing a TCR or a CAR targeting the WT1/HLA-A2+ complex. In another exemplary embodiment, a panel of primary cells and established cell lines of different tissue origins are engineered to stably express NLuc or LucPPe-146-1H2 or other suitable luciferases described in this invention. The primary cells or cell lines could be further genetically modified to lose the expression of specific genes and proteins. The panel is then used to monitor the on-target and off-target toxicities of candidate agents (e.g. CAR-T cells, TCR-T cells or bispecific antibodies) against different cells, tissues and target antigens to select the best candidate for further clinical development.

### Example 43

### Utility of the Matador Cytotoxicity Assay to Demonstrate Cytotoxicity of a Number of Candidate CAR Constructs

T cells were engineered to express CARs, including chimeric TCRs, targeting more than 100 antigens. The target antigens of the CARs included cell surface proteins as well as intracellular proteins. The target antigens of the CARs also included viral proteins, such as HIV1 cell surface glycoprotein. To test the cytotoxic activity of these CAR-T cells, a panel of hematopoietic- and solid tumor-derived cell lines, (Table 3), were engineered to stably express intracellularly luciferase reporters, such as GLuc, NLuc, PaLuc or TLuc. The CAR-T cells were co-cultured with the cell line(s) expressing their cognate target antigen(s) for 4 hours to 96 hours at E:T ratios of 1:1 to 1:10 and the cytotoxicity assay conducted as described in the previous examples by addition of CTZ containing assay buffers using an autoinjector. CAR-T cells were shown to result in increase in luciferase activity when co-cultured with the target cell lines as compared to T cells that had not been infected with any CAR-encoding lentivirus or T cells that were engineered to express a non-specific control CAR. Some of the detailed results are included in PCT/US2017/024843 which is incorporated herein by reference. Collectively, these results demonstrate the broad utility of the Matador cytotoxicity assay described here to detect the cytotoxicity of T cells directed against a diverse group of cell surface and intracellular antigens. The results also demonstrate that the Matador cytotoxicity assay can be used to detect the cytotoxicity of a large number of healthy and diseased cells derived from different organs, tissues and infected with different infectious agents. Although in the above examples, CAR-modified T cells were used as the cytotoxic agents, the utility of this assay, however, is not limited to CAR-T cells and the assay can be used to detect the cytotoxic activity of any agent, including but not limited to unmodified T cells, synthetic TCR-expressing T cells and antibodies (including bispecific antibodies, DARTs and antibody drug conjugates etc.).

TABLE 3
Cell lines engineered to express luciferases for measuring cytotoxicity
of different CAR constructs targeting cell surface and intracellular antigens.

| | Culture | |
|---|---|---|
| Cell line | Conditions | CAR-Antigen |
| BC-1 | RPMI, 20% FCS | BCMA, GPRC, CD138 |
| BC-3 | RPMI, 20% FCS | BCMA, GPRC, CD138 |
| BCBL-1 | RPMI, 20% FCS | GPRC, CD138 |
| JSC-1 | RPMI, 20% FCS | GPRC, CD138 |
| MM1S | RPMI, 10% FCS | CD38, GPRC, CD44, CD200R |
| U266 | RPMI, 10% FCS | BCMA, WT1/HLA-A2+, CS1, CLL1, CD138, c-MET, |
| | | IL6R, CD179b, NY-ESO/HLA-A2, NYBR, LAMP1 |
| L363 | RPMI, 10% FCS | BCMA, GPRC, WT1/HLA-A2+, CS1, CLL1, CD138, |
| | | NY-ESO/HLA-A2, NYBR, LAMP1 |
| K562 | RPMI, 10% FCS | CD33, IL1Ra, TnAg |
| BV173 | RPMI, 10% FCS | CD123, CD179b, IL1Ra, WT1/HLA-A2+, CXCR4, FLT3 |
| Nalm6 | RPMI, 10% FCS | CD19, CD20, CD22, CD179b |
| HL60 | RPMI, 10% FCS | CD33, CD34, CLL1, IL6R, CD32, CD179 |
| U937 | RPMI, 10% FCS | CD4, CLL1 |
| RS: 411 | RPMI, 20% FCS | CD19, Folate Receptor beta (FRbeta), TGFbeta, CD179b, |
| | | NKGD2, FLT3 |
| MV: 411 | RPMI, 10% FCS | FLT3, CD123, FRbeta |
| Raji | RPMI, 10% FCS | CD19, CD20, CD22, BCMA, CD38, CD70, CD79, Folate |
| | | Receptor beta, CLL1 |
| HEL-92.1.7 | RPMI, 10% FCS | MPL, CD33, CD32, CD200R |
| Jurkat | RPMI, 10% FCS | TnAg, TSLRP, TSHR, CD4, CD38 |
| Daudi | RPMI, 10% FCS | BCMA, FRbeta |
| REC-1 | RPMI, 10% FCS | NKGD2, ROR1 |
| KG-1 | RPMI, 20% FCS | CD33, CD34, CD123, TSLRP |
| CEM | RPMI, 10% FCS | CD5 |
| U937 | RPMI, 10% FCS | CD4, CLL1 |
| LAMA5 | RPMI, 10% FCS | WT1/HLA-A2 |
| A549 | DMEM, 10% FCS | ROR1, CD22, TIM1, CDH17 |
| HT29 | DMEM, 10% FCS | EGFR, SLEA, c-MET |
| Molm-13 | RPMI, 20% FCS | FLT3, IL6R, LAMP1, TSLRP, CD4, CSF2RA, CXCR4, |
| | | IL6R, CSF2RA, GPC3 |
| A431 | DMEM, 10% FCS | EGFR, Folate Receptor Alpha, Her3 |
| P19 | DMEM, 10% FCS | SSEA |
| THP-1 | RPMI, 10% FCS | CD32, CD33, CXCR4, CD123, CD44, IL6R, Folate |
| | | Receptor beta, CD70, LAMP1, FLT3, CSF2RA |
| U87MG | DMEM, 10% FCS | CD276, gpNMB, IL13RA2 |
| LoVo | DMEM, 10% FCS | Tissue Factor, CDH17, EGFR |
| SKOV-3 | DMEM, 10% FCS | Folate Receptor alpha (FR1), FSHR, Her2, Her3, LHR, |
| | | MSLN, TIM1, EPCAM |
| NCI-H1993 | DMEM, 10% FCS | EGFR |
| Kasumi-1 | RPMI, 20% FCS | CLEC5A, PR1/HLA-A2, TGFbeta, |
| Jeko-1 | RPMI, 20% FCS | BCMA, ROR1 |
| PC-3 | DMEM, 10% FCS | CGH, TROP2, PSCA, PSMA. EPCAM, FSHR |
| HeLa | DMEM, 10% FCS | EGFR, FR1, MSLN, TSHR |
| LnCap | DMEM, 10% FCS | EGFR, FSHR, PSCA, PSMA, CD22, Her3, CD22, LHR |
| OVCAR-3 | DMEM, 10% FCS | B7H4, CDH6, DLL3, FR1, FSH, LHR, MSLN, PTK7, |
| | | TnAg, TSHR, L1CAM |
| MEL-624 | DMEM, 10% FCS | CDH19, GD2, GD3, gp100/HLA-A2, gpNMB, |
| | | HMWMAA, NYESO/HLA-A2, MART1/HLA-A2 |
| LS174-T | DMEM, 10% FCS | CEA |
| MEL-526 | DMEM, 10% FCS | GD2 |
| MDA-MB231 | DMEM, 10% FCS | CD324, Muc1 |
| L1236 | RPMI, 20% FCS | CD30, CD23, PDL1 |
| L428 | RPMI, 20% FCS | CD30, CD123, CCR4, PDL1 |
| L540 | RPMI, 20% FCS | CD30, CCR4, PDL1 |
| Molt-16 | RPMI, 20% FCS | IL1ra, NKGD2 |
| CEM | RPMI, 10% FCS | CD5 |
| MG-63 | DMEM, 10% FCS | IL13RA2 |
| Karpass-299 | RPMI, 20% FCS | Alk, GPRC, PDL1 |
| MCF7 | DMEM, 10% FCS | B7D4, CD276, TROP2, Her3, Muc1, LewisY, LHR |
| AA-2 | RPMI, 10% FCS | HIV1 env glycoprotein (gp120) |
| HL2/3 | DMEM, 10% FCS | HIV1 env glycoprotein (gp120) |
| TF228.1.16 | DMEM, 10% FCS | HIV1 env glycoprotein (gp120), CCR4 |
| TT | DMEM, 10% FCS | TGF-Beta, TSHR, GFRa1pha4 |
| DMS79 | RPMI, 10% FCS | Fucosyl-GM1, Slea (CA19.9; Sialyl Lewis Antigen) |
| LAN-5 | DMEM, 10% FCS | ALK, DLL3, GFRalpha4, GM1 |
| PEER1 | RPMI, 10% FCS | TSHR |
| SK-MEL-37 | DMEM, 10% FCS | DLL3, GD2 |
| F9 | DMEM, 10% FCS | SSEA |
| HepG2 | DMEM, 10% FBS | GPC3, AFP/HLA-A2 |

### Example 44

### Use of Non-Secretory Form of Human Placental Alkaline Phosphatase in Matador Cytotoxicity Assay

A gene fragment encoding human embryonal alkaline phosphatase lacking its secretory sequence was codon optimized (SEQ ID NO: 27) and cloned into MSCV and lentiviral vectors. 293FT cells were transfected in 24-well plates with 1) a control plasmid (pCDNA3), 2) a retroviral vector (MSCV) encoding human embryonal alkaline phosphatase lacking its secretory sequence; 3) a lentiviral vector encoding human embryonal alkaline phosphatase lacking its secretory sequence; 4) pCMV-SEAP vector encoding secretory form of human embryonal alkaline phosphatase. Each construct was transfected in 6 wells at 500 ng/well. The culture medium was changed from DMEM 10% FCS to XVIVO medium before transfection due to the concern that phenol red in the DMEM medium could interfere with subsequent performance of the assay to detect alkaline phosphatase. 18 hours post transfection, 3 well were left untreated and three wells were treated with Digitonin for 90 min. Cells were scraped and collected in 1.5 ml tubes. An aliquot was centrifuged to collect supernatant only fraction. 10 μl of the supernatant fraction was plated in triplicate in a 384 well plate. A PNPP tablet ((p-nitrophenyl phosphate; Sigma-Aldrich) was dissolved in 20 ml of water as directed by manufacturer and used as substrate to measure alkaline phosphatase activity. 50 μl of substrate was added to each well containing the supernatant. Plates were mixed and incubated at room temp to develop the reaction. Absorbance was measured at 405 nm using a BioTek synergy plate reader. FIG. 40 shows greater than 3-fold increase in alkaline phosphatase activity in the cell supernatants upon induction of cell death by Digitonin in cells that had been transfected with the constructs encoding human embryonal alkaline phosphatase lacking the secretory signal while there was little or no increase in the alkaline phosphatase activity upon Digitonin treatment in cells that had been transfected with an empty vector or a plasmid encoding the secretory form of human embryonal alkaline phosphatase.

### Example 45

### Use of In Vitro Transcribed (IVT) RNA for Expression of GLuc in Matador Assay

IVT to generate GLuc encoding RNAs is performed essentially as described (Zhao Y, et al, MOLECULAR THERAPY Vol. 13, No. 1, 2006). The mMESSAGE mMACHINE High Yield Capped RNA Transcription Kit (Invitrogen) is utilized to generate IVT RNA using GLuc cDNA cloned in the pCDNA3 vector as a template. The IVT RNA is purified using an RNeasy Mini Kit (Qiagen, Inc., Valencia, CA, USA) and purified RNA is eluted in RNase-free water at 1-0.5 μg/ml. For the electroporation, RAJI cells (0.1 ml) are electroporated with 5 g of RNA encoding GLuc. Cells and cuvettes are pre-chilled by putting them on ice for 5 min before electroporation. Subsequently, approximately 0.1 ml of the cells (106 cells) are mixed with RNA and electroporated in a 2-mm cuvette (Harvard Apparatus BTX, Holliston, MA, USA), using an ECM830 Electro Square Wave Porator (Harvard Apparatus BTX). Immediately after electroporation, the cells are transferred to fresh media and incubated at 37° C. After 48 hours, the cells transfected with IVT RNA encoding GLuc are used in Matador assay after co-culture with T cells expressing a CAR directed against CD19 (SEQ ID NO: 80) at an E:T ratio of 10:1 in a 384 well plate. At the end of the co-culture period, GLuc luminescence is measured by adding CTZ-containing assay buffer. Co-culture of RAJI cells transfected with Gluc IVT RNA with CD19 CAR-T cells is shown to result in increase in GLuc activity as compared to cells that are co-cultured with a control CAR-expressing T cells or T cells that do not express any CAR.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

### REFERENCES

1. Bovenberg, M. S., M. H. Degeling and B. A. Tannous (2012). “Enhanced Gaussia luciferase blood assay for monitoring of in vivo biological processes.” Anal Chem 84(2): 1189-1192.
2. Brown, C. E., C. L. Wright, A. Naranjo, R. P. Vishwanath, W. C. Chang, S. Olivares, J. R. Wagner, L. Bruins, A. Raubitschek, L. J. Cooper and M. C. Jensen (2005). “Biophotonic cytotoxicity assay for high-throughput screening of cytolytic killing.” J Immunol Methods 297(1-2): 39-52.
3. Degeling, M. H., M. S. Bovenberg, G. K. Lewandrowski, M. C. de Gooijer, C. L. Vleggeert-Lankamp, M. Tannous, C. A. Maguire and B. A. Tannous (2013). “Directed molecular evolution reveals Gaussia luciferase variants with enhanced light output stability.” Anal Chem 85(5): 3006-3012.
4. Eshhar, Z., T. Waks, G. Gross and D. G. Schindler (1993). “Specific activation and targeting of cytotoxic lymphocytes through chimeric single chains consisting of antibody-binding domains and the gamma or zeta subunits of the immunoglobulin and T-cell receptors.” Proc Natl Acad Sci USA 90(2): 720-724.
5. Fu, X., L. Tao, A. Rivera, S. Williamson, X. T. Song, N. Ahmed and X. Zhang (2010). “A simple and sensitive method for measuring tumor-specific T cell cytotoxicity.” PLoS One 5(7): e11867.
6. Gross, G., Z. Eshhar, G. S and J. CH (2016). “Therapeutic Potential of T Cell Chimeric Antigen Receptors (CARs) in Cancer Treatment: Counteracting Off-Tumor Toxicities for Safe CAR T Cell Therapy
7. Going viral: chimeric antigen receptor T-cell therapy for hematological malignancies.” Annual Review of Pharmacology and Toxicology 56(1): 59-83.
8. Gross, G., T. Waks and Z. Eshhar (1989). “Expression of immunoglobulin-T-cell receptor chimeric molecules as functional receptors with antibody-type specificity.” Proc Natl Acad Sci USA 86(24): 10024-10028.
9. Hall, M. P., J. Unch, B. F. Binkowski, M. P. Valley, B. L. Butler, M. G. Wood, P. Otto, K. Zimmerman, G. Vidugiris, T. Machleidt, M. B. Robers, H. A. Benink, C. T. Eggers, M. R. Slater, P. L. Meisenheimer, D. H. Klaubert, F. Fan, L. P. Encell and K. V. Wood (2012). “Engineered luciferase reporter from a deep sea shrimp utilizing a novel imidazopyrazinone substrate.” ACS Chem Biol 7(11): 1848-1857.
10. Inouye, S. and Y. Sahara (2008). “Identification of two catalytic domains in a luciferase secreted by the copepod Gaussia princeps.” Biochem Biophys Res Commun 365(1): 96-101.
11. Matta, H., B. Hozayev, R. Tomar, P. Chugh and P. M. Chaudhary (2003). “Use of lentiviral vectors for delivery of small interfering RNA.” Cancer Biol Ther 2(2): 206-210.
12. Roybal, K. T., L. J. Rupp, L. Morsut, W. J. Walker, K. A. McNally, J. S. Park and W. A. Lim (2016). “Precision Tumor Recognition by T Cells With Combinatorial Antigen-Sensing Circuits.” Cell 164(4): 770-779.
13. Schafer, H., A. Schafer, A. F. Kiderlen, K. N. Masihi and R. Burger (1997). “A highly sensitive cytotoxicity assay based on the release of reporter enzymes, from stably transfected cell lines.” J Immunol Methods 204(1): 89-98.
14. Sharel, A., A. Adamo, R. Langer and K. F. Jansen Intracellular delivery. W. I. Property, Massachusetts Institute of Technology: 1-126.
15. Takenaka, Y., A. Yamaguchi, N. Tsuruoka, M. Torimura, T. Gojobori and Y. Shigeri (2012). “Evolution of bioluminescence in marine planktonic copepods.” Mol Biol Evol 29(6): 1669-1681.
16. Teeling, J. L., R. R. French, M. S. Cragg, J. van den Brakel, M. Pluyter, H. Huang, C. Chan, P. W. Parren, C. E. Hack, M. Dechant, T. Valerius, J. G. van de Winkel and M. J. Glennie (2004). “Characterization of new human CD20 monoclonal antibodies with potent cytolytic activity against non-Hodgkin lymphomas.” Blood 104(6): 1793-1800.
17. Thompson, J. F., L. S. Hayes and D. B. Lloyd (1991). “Modulation of firefly luciferase stability and impact on studies of gene regulation.” Gene 103(2): 171-177.
18. Thorne, N., J. Inglese and D. S. Auld (2010). “Illuminating insights into firefly luciferase and other bioluminescent reporters used in chemical biology.” Chem Biol 17(6): 646-657.
19. van Rijn, S., J. Nilsson, D. P. Noske, W. P. Vandertop, B. A. Tannous and T. Wurdinger (2013). “Functional multiplex reporter assay using tagged Gaussia luciferase.” Sci Rep 3: 1046.
20. van Rijn, S., T. Wurdinger and J. Nilsson (2014). “Multiplex functional bioluminescent reporters using Gaussia luciferase fused to epitope tags in an immunobinding assay.” Methods Mol Biol 1098: 231-247.
21. Welsh, J. P., K. G. Patel, K. Manthiram and J. R. Swartz (2009). “Multiply mutated Gaussia luciferases provide prolonged and intense bioluminescence.” Biochem Biophys Res Commun 389(4): 563-568.

I claim:

1. A method for assessing cytotoxicity of an agent comprising:
i) exposing a target cell that has been engineered to express intracellularly a non-secretory reporter to an agent capable of modulating cytotoxicity;
ii) measuring the level of the reporter;
wherein the method has the sensitivity to detect the cytotoxicity at the level of a single cell.

2. The method of claim 1, where the method has the sensitivity to detect the cytotoxicity at the level of a) 1000 cells; b) 5000 cells; or c) 10,000 cells.

3. The method of claim 1, where the reporter possesses one or more features selected from the group consisting of
a) reporter is thermostable;
b) reporter is serum-stable;
c) reporter is a fusion protein;
d) reporter is non-toxic to the target cell;
e) reporter is monomeric;
f) reporter is a luciferase or homologs or orthologs or mutants or variants or derivatives thereof;
g) reporter is derived from a copepod or a deep-sea shrimp;
h) reporter can interact with a substrate or a cofactor to generate light, and wherein optionally the substrate is furimazine, coelentrazine or D-luciferin or a synthetic analog thereof;

4. The method of claim 1, wherein the reporter is stable under the assay conditions at 37° C. for more than 15 min.

5. The method of claim 1, wherein the reporter is any one or more of Photuris pennsylvanica luciferase (LucPPe)-146-1H2, LucPPe-133-1B2, LucPPe-78-0B10, LucPPe49-7C6A, Pyrophorus plagiophthalanus luciferase (LucPpL)-81-6G1, Gaussia princeps luciferase (GLuc), Metrida longa luciferase-7 (MLuc7), Heterorhabdus tanneri luciferase (HTLuc), Pleuromamma abdominalis luciferase-1 (PaLuc1), Pleuromamma abdominalis luciferase-2 (PaLuc2), Metridia pacifica luciferase-1 (MpLuc1), Metridia curticauda luciferase-1 (McLuc1), Metridia asymmetrica luciferase-1 (MaLuc1), Metridia okhotensis luciferase-1 (MoLuc1), Metridia okhotensis luciferase-2 (MoLuc2), Metrida longa luciferase-39 (MLuc39), Pleuromamma scutullata luciferase-1 (PsLuc1), Lucicutia ovaliformis luciferase 1-3 (LoLuc1-3), Heterorhabdus tanneri luciferase-2 (HtLuc2), TurboLuc16 (TLuc), Lucia Luc, Renilla Luc, Firefly luciferase (FfLuc or Fluc), NanoLuc (NLuc), or CBGRluc or homologs or orthologs or mutants or variants or derivatives thereof.

6. The method of claim 1, wherein the reporter activity is measured by the addition of a substrate and wherein optionally the substrate is furimazine, coelentrazine or D-luciferin or a synthetic analog thereof with which the reporter can interact to generate light.

7. The method of claim 1, wherein the target cells are present in a cell media and wherein the reporter is assayed in a) the cell media containing the target cells; or b) cell supernatant that is free of the target cells.

8. The method of claim 1, where the method is a single-step homogenous method.

9. The method of claim 1, where the method is performed in a multi-well plate, wherein optionally the multi-well plate is a 96- or a 384-well plate.

10. The method of claim 1, wherein a change in the reporter activity relative to a reference value is indicative of the agent being able to modulate cytotoxicity of the target cell, and wherein optionally the reference value is the reporter activity in any one or more of (i) target cells that do not express reporter, (ii) target cells that express reporter but are not treated with the test agent(s); (iii) the target cells that are left untreated; (iv) target cells that are not treated with the substrate for the reporter, (v) target cells that express the reporter but do not express the target of the agent or (vi) a combination thereof.

11. The method of claim 1, wherein the agent capable of modulating cytotoxicity is any one or more of
a) an antibody, wherein optionally the antibody is any one or more of chimeric antibodies, human antibodies, humanized antibodies, bispecific antibodies, bispecific T cell engager, Dual Affinity Re-targeting (DART), antibody drug conjugates or combination thereof.
b) small molecule,
c) chemical compound,
d) radiation agent,
e) cytotoxic cells, wherein optionally the cytotoxic cells are any one or more of thymus lymphocytes (T cells), Natural Killer (NK) cells, peripheral blood mononuclear cells (PBMCs) or combinations thereof, and wherein optionally the cytototoxic cells are modified to express a chimeric antigen receptor, a synthetic receptor or a T cell receptor.
f) biologics.

12. The method of claim 11, wherein the cytotoxic cells are any one or more of thymus lymphocytes (T cells), Natural Killer (NK) cells, peripheral blood mononuclear cells (PBMCs) or combinations thereof, and wherein optionally the cytototoxic cells are modified to express a chimeric antigen receptor, a synthetic receptor or a T cell receptor.

13. The method of claim 1, where the agent capable of modulating cytotoxicity targets an antigen, wherein the antigen is selected from the group consisting of: Cluster of Differentiation (CD)-19 (CD19); CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRviii); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms Like Tyrosine Kinase 3 (FLT3); Tumor-associated glycoprotein 72 (TAG72); CD38; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3; transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family Member 1A (XAGE-1A); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCT A-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); lymphocyte-specific protein tyrosine kinase (LCK); A kinase anchor protein 4 (AKAP-4); synovial sarcoma X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); immunoglobulin lambda-like polypeptide 1 (IGLL1); MPL; Biotin; c-MYC epitope Tag; CD34; LAMP1; TROP2; GFRalpha4; CDH17; CDH6; NYBR1; CDH19; CD200R; Slea (CA19.9; Sialyl Lewis Antigen) Fucosyl-GM1; PTK7; gpNMB; CDH1; DLL3; CD276/B7H3; IL11Ra; IL13Ra2; CD179b; ALK; TCR gamma-delta; NKG2D; CD32 (FCGR2A); Tim1; CSF2RA (GM-CSFR-alpha); TGFbetaR2; VEGFR2/KDR; Lewis Ag; TCR-beta1 chain; TCR-beta2 chain; TCR-gamma chain; TCR-delta chain; FITC; Luteinizing hormone receptor (LHR); Follicle stimulating hormone receptor (FSHR); Chorionic Gonadotropin Hormone receptor (CGHR); CCR4; GD3; SLAMF6; SLAMF4; HIV1 envelope glycoprotein; HTLV1-Tax; CMV pp65; EBV-EBNA3c; influenza A hemagglutinin (HA); GAD; PDL1; Guanylyl cyclase C (GCC); KSHV-K8.1 protein; KSHV-gH protein; HLA; HLA-A; HLA-A2; HLA-B; HLA-C; HLA-DP; HLA-DM; HLA-DOA; HLA-DOB; HLA-DQ; HLA-DR or HLA-G.

14. The method of claim 1, wherein the target cell expresses one or more of the antigens selected from a group consisting of: Cluster of Differentiation (CD)-19 (CD19); CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRviii); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms Like Tyrosine Kinase 3 (FLT3); Tumor-associated glycoprotein 72 (TAG72); CD38; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3; transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family Member 1A (XAGE-1A); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCT A-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); lymphocyte-specific protein tyrosine kinase (LCK); A kinase anchor protein 4 (AKAP-4); synovial sarcoma X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); immunoglobulin lambda-like polypeptide 1 (IGLL1); MPL; Biotin; c-MYC epitope Tag; CD34; LAMP1; TROP2; GFRalpha4; CDH17; CDH6; NYBR1; CDH19; CD200R; Slea (CA19.9; Sialyl Lewis Antigen) Fucosyl-GM1; PTK7; gpNMB; CDH1; DLL3; CD276/B7H3; IL11Ra; IL13Ra2; CD179b; ALK; TCR gamma-delta; NKG2D; CD32 (FCGR2A); Tim1; CSF2RA (GM-CSFR-alpha); TGFbetaR2; VEGFR2/KDR; Lewis Ag; TCR-beta1 chain; TCR-beta2 chain; TCR-gamma chain; TCR-delta chain; FITC; Luteinizing hormone receptor (LHR); Follicle stimulating hormone receptor (FSHR); Chorionic Gonadotropin Hormone receptor (CGHR); CCR4; GD3; SLAMF6; SLAMF4; HIV1 envelope glycoprotein; HTLV1-Tax; CMV pp65; EBV-EBNA3c; influenza A hemagglutinin (HA); GAD; PDL1; Guanylyl cyclase C (GCC); KSHV-K8.1 protein; KSHV-gH protein; HLA; HLA-A; HLA-A2; HLA-B; HLA-C; HLA-DP; HLA-DM; HLA-DOA; HLA-DOB; HLA-DQ; HLA-DR or HLA-G.

15. The method of claim 1, where the method further comprises assessing cytotoxicity of the agent on a negative control cell or a cell line comprising:
i) exposing the negative control cell or cell line that has been engineered to express intracellularly a non-secretory reporter to an agent capable of modulating cytotoxicity;
ii) measuring the level of the reporter; and
wherein the negative control cell or cell line is modified and/or selected to lacks the expression of one or more antigens selected from the group consisting of: Cluster of Differentiation (CD)-19 (CD19); CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRviii); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms Like Tyrosine Kinase 3 (FLT3); Tumor-associated glycoprotein 72 (TAG72); CD38; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3; transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family Member 1A (XAGE-1A); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCT A-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); lymphocyte-specific protein tyrosine kinase (LCK); A kinase anchor protein 4 (AKAP-4); synovial sarcoma X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); immunoglobulin lambda-like polypeptide 1 (IGLL1); MPL; Biotin; c-MYC epitope Tag; CD34; LAMP1; TROP2; GFRalpha4; CDH17; CDH6; NYBR1; CDH19; CD200R; Slea (CA19.9; Sialyl Lewis Antigen) Fucosyl-GM1; PTK7; gpNMB; CDH1; DLL3; CD276/B7H3; IL11Ra; IL13Ra2; CD179b; ALK; TCR gamma-delta; NKG2D; CD32 (FCGR2A); Tim1; CSF2RA (GM-CSFR-alpha); TGFbetaR2; VEGFR2/KDR; Lewis Ag; TCR-beta1 chain; TCR-beta2 chain; TCR-gamma chain; TCR-delta chain; FITC; Luteinizing hormone receptor (LHR); Follicle stimulating hormone receptor (FSHR); Chorionic Gonadotropin Hormone receptor (CGHR); CCR4; GD3; SLAMF6; SLAMF4; HIV1 envelope glycoprotein; HTLV1-Tax; CMV pp65; EBV-EBNA3c; influenza A hemagglutinin (HA); GAD; PDL1; Guanylyl cyclase C (GCC); KSHV-K8.1 protein; KSHV-gH protein; HLA; HLA-A; HLA-A2; HLA-B; HLA-C; HLA-DP; HLA-DM; HLA-DOA; HLA-DOB; HLA-DQ; HLA-DR or HLA-G.

16. The method of claim 1, which is practiced using a kit containing one or more components of claim 1.

17. A cell or a cell line that has been engineered to
a) express intracellularly a non-secretory reporter; and
b) lack the expression of one or more antigens, wherein optionally the one or more antigens are selected from the group consisting of: Cluster of Differentiation (CD)-19 (CD19); CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRviii); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms Like Tyrosine Kinase 3 (FLT3); Tumor-associated glycoprotein 72 (TAG72); CD38; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3; transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family Member 1A (XAGE-1A); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCT A-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); lymphocyte-specific protein tyrosine kinase (LCK); A kinase anchor protein 4 (AKAP-4); synovial sarcoma X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); immunoglobulin lambda-like polypeptide 1 (IGLL1); MPL; Biotin; c-MYC epitope Tag; CD34; LAMP1; TROP2; GFRalpha4; CDH17; CDH6; NYBR1; CDH19; CD200R; Slea (CA19.9; Sialyl Lewis Antigen) Fucosyl-GM1; PTK7; gpNMB; CDH1; DLL3; CD276/B7H3; IL11Ra; IL13Ra2; CD179b; ALK; TCR gamma-delta; NKG2D; CD32 (FCGR2A); Tim1; CSF2RA (GM-CSFR-alpha); TGFbetaR2; VEGFR2/KDR; Lewis Ag; TCR-beta1 chain; TCR-beta2 chain; TCR-gamma chain; TCR-delta chain; FITC; Luteinizing hormone receptor (LHR); Follicle stimulating hormone receptor (FSHR); Chorionic Gonadotropin Hormone receptor (CGHR); CCR4; GD3; SLAMF6; SLAMF4; HIV1 envelope glycoprotein; HTLV1-Tax; CMV pp65; EBV-EBNA3c; influenza A hemagglutinin (HA); GAD; PDL1; Guanylyl cyclase C (GCC); KSHV-K8.1 protein; KSHV-gH protein; HLA; HLA-A; HLA-A2; HLA-B; HLA-C; HLA-DP; HLA-DM; HLA-DOA; HLA-DOB; HLA-DQ; HLA-DR or HLA-G

18. The cell or cell line of claim 17, wherein the reporter has one or more of the features selected from the group consisting of
a) reporter is thermostable;
b) reporter is serum-stable;
c) reporter is a fusion protein;
d) reporter is non-toxic to the cell or cell line;
e) reporter is monomeric;
f) reporter is a luciferase or homologs or orthologs or mutants or variants or derivatives thereof;
g) reporter is derived from a copepod or a deep-sea shrimp;
h) reporter can interact with a substrate or a cofactor to generate light, and wherein optionally the substrate is furimazine, coelentrazine or D-luciferin or a synthetic analog thereof,

19. A single step method for assessing cytotoxicity of an agent comprising:
i) exposing a target cell that has been engineered to express intracellularly a non-secretory reporter to an agent capable of modulating cytotoxicity;
ii) measuring the level of the reporter by the addition of an Imidazopyrazinone substrate, wherein optionally the Imidazopyrazinone substrate is furimazin or a derivative thereof
wherein the method has the sensitivity to detect the cytotoxicity at the level of a single cell.

20. The method of claim 19, where the reporter possesses one or more features selected from the group consisting of
a) reporter is thermostable;
b) reporter is serum-stable;
c) reporter is a fusion protein;
d) reporter is non-toxic to the target cell;
e) reporter is monomeric;
f) reporter is a luciferase or homologs or orthologs or mutants or variants or derivatives thereof;
g) reporter is derived from a copepod or a deep-sea shrimp;
h) reporter can interact with a substrate or a cofactor to generate light, and wherein optionally the substrate is furimazine.
